(19) United States
(12) Patent Application Publication
(10) Pub. No.: US 2017/0022499 A1
(43) Pub. Date: Jan. 26, 2017

(54) METHODS AND COMPOSITIONS FOR THE PRODUCTION OF GUIDE RNA

(71) Applicant: Massachusetts Institute of Techology, Cambridge, MA (US)

(72) Inventors: Timothy Kuan-Ta Lu, Cambridge, MA (US); Lior Nissim, Cambridge, MA (US); Samuel David Perli, Cambridge, MA (US)

(73) Assignee: Massachusetts Institute of Techology, Cambridge, MA (US)

(21) Appl. No.: 15/301,135

(22) PCT Filed: Apr. 3, 2015
(86) PCT No.: PCT/US2015/024196

§ 371 Date: Sep. 30, 2016

Related U.S. Application Data
(60) Provisional application No. 61/974,672, filed on Apr. 3, 2014.

(51) Int. Cl. C12N 15/113; C12N 9/22; C12N 15/85

(52) U.S. Cl. CPC C12N 15/113; C12N 15/85; C12N 9/22; C12Y 301/00; C12N 2310/10; C12N 2310/128; C12N 2330/51; C12N 2310/141

(57) ABSTRACT

Various aspects and embodiments of the present disclosure relate to methods and compositions that combine multiple mammalian RNA regulatory strategies, including RNA triple helix structures, introns, microRNAs, and ribozymes with Cas-based CRISPR transcription factors and ribonuclease-based RNA processing in human cells. The methods and compositions of the present disclosure, in some embodiments, enable multiplexed production of proteins and multiple guide RNAs from a single compact RNA-polymerase-II-expressed transcript for efficient modulation of synthetic constructs and endogenous human promoters.

### RELATED APPLICATIONS

This application claims the benefit under 35 U.S.C. §119(e) of U.S. provisional application No. 61/974,672, filed Apr. 3, 2014, which is incorporated by reference herein in its entirety.

### FEDERALLY SPONSORED RESEARCH

This invention was made with government support under Contract No. W911NF-11-2-0056 awarded by the Army Research Office. The government has certain rights in the invention.

### FIELD OF THE INVENTION

Aspects of the present disclosure relate to biotechnology. In particular, some embodiments are directed to the fields of transcriptional regulation and synthetic biology.

### BACKGROUND OF INVENTION

Recently, bacterial type II CRISPR/Cas systems (clustered, regularly interspaced, short palindromic repeats (CRISPR)/CRISPR associate system (Cas)) have been adapted to achieve programmable DNA binding without requiring complex protein engineering. Cas proteins are nucleases specialized for cutting DNA. In the type II CRISPR/Cas systems, the sequence specificity of the Cas DNA-binding protein is determined by guide RNAs (gRNAs), which have nucleotide base-pairing complementarity to target DNA sites. This enables simple and highly flexible programming of Cas binding.

### SUMMARY OF INVENTION

A major challenge in constructing CRISPR-based circuits in mammalian cells (e.g., human cells), especially those that interface with endogenous promoters, is that multiple gRNAs are often necessary to achieve desired activation levels. Current techniques rely on the use of multiple gRNA expression constructs, each with their own promoter. The engineered constructs described herein, in some embodiments, can be used to express many functional gRNAs from a single transcript, thus enabling compact encoding of synthetic gene circuits with multiple outputs as well as concise strategies for modulating native genes and rewiring native networks. Thus, provided herein, in some embodiments, are methods and compositions (e.g., nucleic acids and cells) that enable production of scalable synthetic gene circuits and/or modification of endogenous genes and gene networks by integrating ribonucleic acid (RNA)-based regulatory mechanisms, such as RNA interference and CRISPR/Cas systems. For example, various embodiments herein combine multiple mammalian RNA regulatory strategies, including RNA triple helix structures, introns, microRNAs and ribozymes, with bacterial Cas-based CRISPR transcription factors (CRISPR-TFs) and ribonuclease-based (e.g., Cas6/Csy4-based) RNA processing in human cells to modify gene expression. Surprisingly, complementary methods of the present disclosure enable expression of functional gRNAs from transcripts generated by RNA polymerase II (RNA pol II, or RNAP II) promoters while permitting co-expression of a protein of interest. Further, the genetic constructs provided herein enable multiplexed expression of proteins and/or RNA interference molecules (e.g., microRNA) with multiple gRNAs, in some embodiments, from a single transcript for efficient modulation of synthetic constructs and endogenous human promoters.

Engineered constructs provided herein are useful, for example, for implementing tunable synthetic gene circuits, including multistage transcriptional cascades. Moreover, the methods and compositions of the present disclosure can be used, in some embodiments, to rewire regulatory connections in RNA-dependent gene circuits with multiple outputs and feedback loops to achieve complex functional behaviors. Engineered constructs provided herein are valuable for the construction of scalable gene circuits and the modification (e.g., perturbation) of natural regulatory networks in, for example, human cells for basic biology, therapeutic and synthetic-biology applications.

Various aspects of the present disclosure provide engineered constructs comprising a promoter operably linked to a nucleic acid that comprises (a) a nucleotide sequence encoding at least one guide RNA (gRNA), and (b) one or more nucleotide sequences selected from (i) a nucleotide sequence encoding a protein of interest and (ii) a nucleotide sequence encoding an RNA interference molecule. In some embodiments, the promoter is a RNA-polymerase-II-dependent (RNA pol II) promoter.

In some embodiments, at least one gRNA is flanked by nucleotide sequences encoding ribonuclease recognition sites. The ribonuclease recognition sites may be, for example, Csy4 ribonuclease recognition sites.

In some embodiments, at least one gRNA is flanked by nucleotide sequences encoding ribozymes. The ribozymes may be selected, for example, from a hammerhead ribozyme and a Hepatitis delta virus ribozyme.

In some embodiments, the nucleotide sequence of (a) is flanked by cognate intronic splice sites.

Some aspects of the present disclosure provide engineered constructs comprising a promoter operably linked to a nucleic acid that comprises a first nucleotide sequence encoding at least one guide RNA (gRNA) flanked by ribonuclease recognition sites. In some embodiments, the promoter is a RNA-polymerase-II-dependent (RNA pol II) promoter. The RNA pol II promoter may be, for example, a human cytomegalovirus promoter, a human ubiquitin promoter, a human histone H2A1 promoter, or a human inflammatory chemokine CXCL1 promoter.

In some embodiments, the first nucleotide sequence is flanked by cognate intronic splice sites.

In some embodiments, the nucleic acid further comprises a second nucleotide sequence encoding a protein of interest. The first nucleotide sequence may be within the second nucleotide sequence, or the second nucleotide sequence may be upstream of the first nucleotide sequence.

In some embodiments, the engineered constructs further comprise a nucleotide sequence encoding at least one microRNA. A microRNA may be, for example, encoded within the protein of interest.

In some embodiments, the nucleic acid further comprises a third nucleotide sequence encoding a triple helix structure, wherein the third nucleotide sequence is between the second nucleotide sequence and the first nucleotide sequence.

In some embodiments, the first nucleotide sequence encodes at least two, at least three, at least four, at least five, or more, gRNAs, each gRNA flanked by ribonuclease recognition sites.

In some embodiments, the first nucleotide sequence encodes at least two gRNAs flanked by ribonuclease recognition sites, and wherein the gRNAs are different from each other.

In some embodiments, the ribonuclease recognition sites are Csy4 ribonuclease recognition sites. Each of the Csy4 ribonuclease recognition sites may have, for example, a length of 28 nucleotides. In some embodiments, the Csy4 ribonuclease recognition sites are from Pseudomonas aeruginosa.

In some embodiments, the triple helix structure is encoded by a nucleotide sequence from the 3′ end of the MALAT1 locus or the 3′ end of the MENβ locus.

Some aspects of the present disclosure provide engineered constructs comprising a promoter operably linked to a nucleic acid that comprises a first nucleotide sequence encoding a protein of interest, and a second nucleotide sequence encoding at least one guide RNA (gRNA) flanked by ribonuclease recognition sites, wherein the second nucleotide sequence is flanked by nucleotide sequences encoding cognate intronic splice sites and is within the first nucleotide sequence. In some embodiments, the promoter is a RNA-polymerase-II-dependent (RNA pol II) promoter. The RNA pol II promoter may be, for example, a human cytomegalovirus promoter, a human ubiquitin promoter, a human histone H2A1 promoter, or a human inflammatory chemokine CXCL1 promoter.

In some embodiments, the engineered constructs further comprise a nucleotide sequence encoding at least one microRNA. A microRNA may, for example, be encoded within the protein of interest.

In some embodiments, the nucleic acid further comprises a third nucleotide sequence encoding a triple helix structure, and a fourth nucleotide sequence encoding at least one gRNA flanked by ribonuclease recognition sites, wherein the third nucleotide sequence is downstream of the first nucleotide sequence and is upstream of the fourth nucleotide sequence.

In some embodiments, the second nucleotide sequence encodes at least two, at least three, at least four, at least five, or more, gRNAs, each gRNA flanked by ribonuclease recognition sites.

In some embodiments, the second nucleotide sequence encodes at least two gRNAs flanked by ribonuclease recognition sites, and wherein the gRNAs are different from each other.

In some embodiments, the ribonuclease recognition sites are Csy4 ribonuclease recognition sites. The Csy4 ribonuclease recognition sites may have, for example, a length of 28 nucleotides. In some embodiments, the Csy4 ribonuclease recognition sites are from Pseudomonas aeruginosa.

In some embodiments, the cognate intronic splice sites are from a consensus intron. In some embodiments, the cognate intronic splice sites are from a HSV1 latency-associated intron. In some embodiments, the cognate intronic splice sites are from a sno-IncRNA2 intron.

In some embodiments, the triple helix structure is encoded by a nucleotide sequence from the 3′ end of the MALAT1 locus or the 3′ end of the MENβ locus.

In some embodiments, the fourth nucleotide sequence encodes at least two, at least three, at least four, at least five, or more, gRNAs, each gRNA flanked by ribonuclease recognition sites.

In some embodiments, the fourth nucleotide sequence encodes at least two gRNAs flanked by ribonuclease recognition sites, and wherein the gRNAs are different from each other.

Some aspects of the present disclosure provide engineered constructs comprising a promoter operably linked to a nucleic acid that comprises a first nucleotide sequence encoding at least one guide RNA (gRNA) flanked by ribozymes. In some embodiments, the promoter is a RNA-polymerase-II-dependent (RNA pol II) promoter. The RNA pol II promoter may be, for example, a human cytomegalovirus promoter, a human ubiquitin promoter, a human histone H2A1 promoter, or a human inflammatory chemokine CXCL1 promoter.

In some embodiments, the nucleic acid further comprise a second nucleotide sequence encoding a protein of interest, wherein the second nucleotide sequence is upstream of the first nucleotide sequence.

In some embodiments, the engineered constructs further comprise a nucleotide sequence encoding at least one microRNA. A microRNA may, for example, be encoded within the protein of interest.

In some embodiments, the nucleic acid further comprises a third nucleotide sequence encoding a triple helix structure, wherein the third nucleotide sequence is between the second nucleotide sequence and the first nucleotide sequence.

In some embodiments, the fourth nucleotide sequence encodes at least two, at least three, at least four, at least five, or more, gRNAs, each gRNA flanked by ribonuclease recognition sites.

In some embodiments, the first nucleotide sequence encodes at least two gRNAs flanked by ribozymes, and wherein the gRNAs are different from each other.

In some embodiments, the ribozymes are cis-acting ribozymes. For example, a cis-acting ribozyme may be a hammerhead ribozyme or a Hepatitis delta virus ribozyme. In some embodiments, a hammerhead ribozyme is at the 5′ end of the at least one gRNA. In some embodiments, a hammerhead ribozyme is at the 3′ end of the at least one gRNA. In some embodiments, a Hepatitis delta virus ribozyme is at the 5′ end of the at least one gRNA. In some embodiments, a Hepatitis delta virus ribozyme is at the 3′ end of the at least one gRNA.

In some embodiments, the triple helix structure is encoded by a nucleotide sequence from the 3′ end of the MALAT1 locus or the 3′ end of the MENβ locus.

Some aspects of the present disclosure provide engineered constructs comprising a promoter operably linked to a nucleic acid that comprises a first nucleotide sequence encoding at least one RNA interference molecule within a protein of interest, a second nucleotide sequence encoding at least one guide RNA flanked by ribonuclease recognition sites, and a third nucleotide sequence encoding a triple helix structure, wherein the third nucleotide sequence is between the first and second nucleotide sequences.

Some aspects of the present disclosure provide engineered constructs comprising a promoter operably linked to a nucleic acid that comprises a first nucleotide sequence encoding at least one RNA interference molecule within a protein of interest, a second nucleotide sequence encoding at least one guide RNA flanked by ribozymes, and a third nucleotide sequence encoding a triple helix structure, wherein the third nucleotide sequence is between the first and second nucleotide sequences.

In some embodiments, an RNA interference molecule is selected from a microRNA (miRNA) and a small-interfering RNA (siRNA). In some embodiments, the at least one RNA interference molecule comprises at least one miRNA.

Some aspects provide vectors comprising one or more of the engineered constructs of the present disclosure.

Some aspects provide cells comprising an engineered constructs of the present disclosure and/or a vector of the present disclosure.

Also provided herein are cells that comprise at least two of the engineered constructs of the present disclosure and/or at least two of the vectors of the present disclosure.

In some embodiments, the cells are modified to stably express a ribonuclease. The ribonuclease may be, for example, a Csy4 ribonuclease.

In some embodiments, the cells are modified to stably express a Cas protein. In some embodiments, the Cas protein is a Cas nuclease such as, for example, a Cas9 nuclease. In some embodiments, the Cas protein is a transcriptionally active Cas protein. In some embodiments, the transcriptionally active Cas protein is a transcriptionally active Cas9 protein.

In some embodiments, the cells further comprise an engineered nucleic acid comprising a promoter operably linked to a nucleotide sequence encoding a ribonuclease. The ribonuclease may be, for example, a Csy4 ribonuclease.

In some embodiments, the cells further comprise an engineered nucleic acid comprising a promoter operably linked to a nucleotide sequence encoding a Cas protein. In some embodiments, the Cas protein is a Cas nuclease such as, for example, a Cas9 nuclease. In some embodiments, the Cas protein is a transcriptionally active Cas protein. In some embodiments, the transcriptionally active Cas protein is a transcriptionally active Cas9 protein.

In some embodiments, the cells further comprise at least one (or at least two) additional engineered nucleic acid comprising a promoter operably linked to a nucleotide sequence encoding a protein of interest. In some embodiments, the protein of interest of an additional engineered nucleic acid is different from any other protein of interest of the cell.

In some embodiments, the cells are bacterial cells. In some embodiments, the cells are human cells.

Also provided herein are methods that comprise culturing any of the cells of the present disclosure. In some embodiments, the methods comprise culturing the cells under conditions that permit nucleic acid expression.

Some aspects of the present disclosure provide methods of producing, modifying or rewiring a cellular genetic circuit, the methods comprising expressing in a cell a first engineered construct selected from any of the engineered construct provided herein, and expressing in the cell a second engineered construct selected from t any of the engineered construct provided herein, wherein at least one gRNA of the first engineered construct is complementary to and binds to a region of the promoter of the second engineered construct or to a region of an endogenous promoter.

In some embodiments, the methods further comprise expressing a third engineered construct selected from any of the engineered construct provided herein, wherein at least one gRNA of the second engineered construct is complementary to and binds to a region of the promoter of the third engineered construct or to a region of an endogenous promoter. In some embodiments, the methods further comprise expressing at least one additional engineered nucleic acid selected from any of the engineered construct provided herein, wherein at least one gRNA of the at least one additional engineered nucleic acid is complementary to and binds to a region of the promoter of any one of the engineered nucleic acids of the cell or to a region of at least one endogenous promoter.

In some embodiments, the cells are modified to stably express a ribonuclease. The ribonuclease may be, for example, a Csy4 ribonuclease.

In some embodiments, the cells are modified to stably express a Cas protein. In some embodiments, the Cas protein is a Cas nuclease such as, for example, a Cas9 nuclease. In some embodiments, the Cas protein is a transcriptionally active Cas protein. In some embodiments, the transcriptionally active Cas protein is a transcriptionally active Cas9 protein.

In some embodiments, the cells further comprise an engineered nucleic acid comprising a promoter operably linked to a nucleotide sequence encoding a ribonuclease. The ribonuclease may be, for example, a Csy4 ribonuclease.

In some embodiments, the cells further comprise an engineered nucleic acid comprising a promoter operably linked to a nucleotide sequence encoding a Cas protein. In some embodiments, the Cas protein is a Cas nuclease such as, for example, a Cas9 nuclease. In some embodiments, the Cas protein is a transcriptionally active Cas protein. In some embodiments, the transcriptionally active Cas protein is a transcriptionally active Cas9 protein.

In Some Embodiments, the Methods Further Comprise Culturing the Cell.

Some aspects of the present disclosure provide methods of multiplexed cellular expression of guide ribonucleic acids (gRNAs) comprising expressing in a cell an engineered construct comprising a promoter operably linked to a nucleic acid that comprises a first nucleotide sequence encoding at least two gRNAs, each gRNA flanked by ribonuclease recognition sites.

In some embodiments, the nucleic acid further comprises a second nucleotide sequence encoding a protein of interest, wherein the second nucleotide sequence is upstream of the first nucleotide sequence.

In some embodiments, the engineered constructs further comprise a nucleotide sequence encoding at least one microRNA. A microRNA may, for example, be encoded within the protein of interest.

In some embodiments, the nucleic acid further comprises a third nucleotide sequence encoding a triple helix structure, wherein the third nucleotide sequence is between the second nucleotide sequence and the first nucleotide sequence.

In some embodiments, the cells are modified to stably express a ribonuclease. The ribonuclease may be, for example, a Csy4 ribonuclease.

In some embodiments, the cells are modified to stably express a Cas protein. In some embodiments, the Cas protein is a Cas nuclease such as, for example, a Cas9 nuclease. In some embodiments, the Cas protein is a transcriptionally active Cas protein. In some embodiments, the transcriptionally active Cas protein is a transcriptionally active Cas9 protein.

In some embodiments, the cells further comprise an engineered nucleic acid comprising a promoter operably linked to a nucleotide sequence encoding a ribonuclease. The ribonuclease may be, for example, a Csy4 ribonuclease.

In some embodiments, the cells further comprise an engineered nucleic acid comprising a promoter operably linked to a nucleotide sequence encoding a Cas protein. In some embodiments, the Cas protein is a Cas nuclease such as, for example, a Cas9 nuclease. In some embodiments, the Cas protein is a transcriptionally active Cas protein. In some embodiments, the transcriptionally active Cas protein is a transcriptionally active Cas9 protein.

In some embodiments, the methods further comprise culturing the cell.

### BRIEF DESCRIPTION OF THE DRAWINGS

FIG. 1A shows an engineered construct, CMVp-mK-Tr-28-g1-28, which includes a CMV promoter (CMVp) operably linked to a nucleic acid that includes a nucleotide sequence encoding an mKate2 protein, which is upstream of a nucleotide sequence encoding a triple helix structure (triplex), which is upstream of a nucleotide sequence encoding a guide RNA (gRNA1) flanked by Csy4 recognition sites (28 bp). The configuration of this engineered construct may be referred to as a ‘triplex/Csy4’ configuration. The schematic in FIG. 1A shows that in cells co-expressing a transcriptionally active form of Cas9 protein (taCas9), Csy4 ribonuclease, CMVp-mK-Tr-28-g1-28, and P1-EYFP, both the mKate2 protein and the guide RNA are expressed. The guide RNA (gRNA) associated with transcriptionally active Cas9 protein to activate a synthetic promoter (P1) driving expression of enhanced yellow fluorescent protein (P1-EYFP).

FIG. 1B shows a graph comparing the level of Csy4 with relative EYFP and mKate2 expression levels from cells co-expressing CMVp-mK-Tr-28-g1-28, Cas9 and Csy4. There is a 60-fold increase in EYFP expression levels, demonstrating the generation of functional gRNAs. Increased concentrations of a Csy4-expressing plasmid led to increased mKate2 expression levels. Fluorescence values were normalized to the maximum respective fluorescence between the data in this figure and in FIGS. 2B-2D to enable cross comparisons between the ‘triplex/Csy4’ and ‘intron/Csy4’ configurations, discussed below.

FIG. 1C shows a graph comparing the effects of Csy4 and Cas9 expression on mKate2 expression levels in cells co-expressing CMVp-mK-Tr-28-g1-28, Csy4 and Cas9. Csy4 and taCas9 have opposite effects on mKate2 fluorescence. The taCas9 construct alone reduced mKate2 levels, while the Csy4 construct alone enhanced mKate2 fluorescence. The mKate2 expression levels were normalized to the maximum mKate2 expression value observed (Csy4 only) across the four conditions tested.

FIG. 1D shows a graph comparing the effects of different RNAP II promoters on relative IL1RN mRNA expression levels. Human RNAP II promoters, CXCL1p, H2A1p and UbCp, as well as the RNAP II promoter, CMVp, were used to drive expression of four different gRNAs (gRNA3-6, Table 1) which activate the IL1RN promoter from a ‘triplex/Csy4’ construct. Results were compared to the effects of the RNAP III promoter, U6p, on direct expression of the same gRNAs. Four different plasmids, each containing one of the indicated promoters and gRNAs 3-6, were co-transfected in cells along with a plasmid encoding taCas9, with or without a plasmid expressing Csy4. Relative IL1RN mRNA expression, compared to a control construct with non-specific gRNA (NS, CMVp-mK-Tr-28-g1-28), was monitored using qRT-PCR. The RNAP II promoters resulted in a wide range of IL1RN activation, with the presence of Csy4 greatly increasing activation compared with the absence of Csy4. IL1RN activation was achieved by the RNAP II promoters even in the absence of Csy4, albeit at much lower levels than in the presence of Csy4.

FIG. 1E shows a graph comparing the input-output transfer curve for the activation of the endogenous IL1RN loci by the ‘triplex/Csy4’ construct, which was determined by plotting mKate2 expression levels (as a proxy for the input) versus relative IL1RN mRNA expression levels (as the output). The data indicated that tunable modulation of endogenous loci can be achieved with RNAP II promoters of different strengths. The IL1RN data is the same as shown in FIG. 1D).

FIG. 2A shows an engineered construct, CMVp-mK_{EX1}-[28-g1-28]ᵢₙₜᵣₒₙ-mK_{EX2}, which includes a CMV promoter (CMVp) operably linked to a nucleic acid that includes a nucleotide sequence encoding a guide RNA (gRNA1) flanked by Csy4 recognition sites (28 bp), which are flanked by cognate intronic splice sites, which are within a nucleotide sequence encoding an mKate2 protein. The configuration of this engineered construct may be referred to as a “intron/Csy4” configuration. The schematic in FIG. 2A shows that in cells co-expressing a transcriptionally active form of Cas9 protein, Csy4 ribonuclease, CMVp-mK_{EX1}-[28-g1-28]ᵢₙₜᵣₒₙ-mK_{EX2}, and P1-EYFP, the guide RNA is expressed, which then associates with transcriptionally active Cas9 protein to activate a synthetic promoter (P1) driving expression of enhanced yellow fluorescent protein (P1-EYFP). In contrast to the ‘triplex/Csy4’ configuration shown in FIG. 1A, with increasing Csy4 levels, the ‘intron/Csy4’ configuration leads to a decrease in expression of the mKate2 gene, which, without being bound by theory, may be due to cleavage of pre-mRNA prior to splicing.

FIG. 2B shows a graph comparing the level of Csy4 with relative EYFP and mKate2 expression levels from cells co-expressing CMVp-mK_{EX1}-[28-g1-28]ᵢₙₜᵣₒₙ-mK_{EX2}, Cas9 and Csy4, where the cognate intronic splice sites are from a consensus intron.

FIG. 2C shows a graph comparing the level of Csy4 with relative EYFP and mKate2 expression levels from cells co-expressing CMVp-mK_{EX1}-[28-g1-28]ᵢₙₜᵣₒₙ-mK_{EX2}, Cas9 and Csy4, where the cognate intronic splice sites are from snoRNA2 intron.

FIG. 2D shows a graph comparing the level of Csy4 with relative EYFP and mKate2 expression levels from cells co-expressing CMVp-mK_{EX1}-[28-g1-28]ᵢₙₜᵣₒₙ-mK_{EX2}, Cas9 and Csy4, where the cognate intronic splice sites are from an HSV1 intron.

FIG. 2E shows a graph comparing the level of Csy4 with relative EYFP and mKate2 expression levels from cells co-expressing CMVp-mK_{EX1}-[28-g1-28]ᵢₙₜᵣₒₙ-mK_{EX2}, Cas9 and Csy4, where a single Csy4 binding site is located upstream of the gRNA within an HSV1 intron. This configuration did not produce functional gRNAs but did lead to reduced mKate2 fluorescence with greater Csy4 levels. The fluorescence values were normalized to the maximum fluorescence levels between this experiment and a [28-g1-28]HSV1 control (FIG. 11).

FIG. 2F shows a graph comparing the level of Csy4 with relative EYFP and mKate2 expression levels from cells co-expressing CMVp-mK_{EX1}-[28-g1-28]ᵢₙₜᵣₒₙ-mK_{EX2}, Cas9 and Csy4, where a single Csy4 binding site is located downstream of the gRNA within an HSV1 intron. This configuration produced low levels of functional gRNA and also generated reduced mKate2 levels with greater Csy4-expressing plasmid concentrations. The fluorescence values were normalized to the maximum fluorescence levels between this experiment and a [28-g1-28]HSV1 control (FIG. 11).

FIG. 3A shows an engineered construct, CMVp-mK-Tr-HH-g1-HDV, which includes a CMV promoter (CMVp) operably linked to a nucleic acid that includes a nucleotide sequence encoding an mKate2 protein, which is upstream of a nucleotide sequence encoding a triple helix structure (triplex), which is upstream of a nucleotide sequence encoding a guide RNA (gRNA1) flanked by ribozymes (5′ hammerhead (HH) ribozyme, and 3′ HDV ribozyme). The configuration of this engineered construct may be referred to as a ‘triplex/ribozyme’ configuration. The schematic in FIG. 3A shows that in cells co-expressing a transcriptionally active form of Cas9 protein, Csy4 ribonuclease, and CMVp-mK-Tr-HH-g1-HDV, both the mKate2 protein and the guide RNA are expressed.

FIG. 3B shows an engineered construct, CMVp-mK-HH-g1-HDV, which includes a CMV promoter (CMVp) operably linked to a nucleic acid that includes a nucleotide sequence encoding an mKate2 protein, which is upstream of a nucleotide sequence encoding a guide RNA (gRNA1) flanked by ribozymes (5′ hammerhead (HH) ribozyme, and 3′ HDV ribozyme). The schematic in FIG. 3B shows that in cells co-expressing a transcriptionally active form of Cas9 protein, Csy4 ribonuclease, and CMVp-mK-HH-g1-HDV, both the mKate2 protein and the guide RNA are expressed.

FIG. 3C shows an engineered construct, CMVp-HH-g1-HDV, which includes a CMV promoter (CMVp) operably linked to a nucleic acid that includes a nucleotide sequence encoding a guide RNA (gRNA1) flanked by ribozymes (5′ hammerhead (HH) ribozyme, and 3′ HDV ribozyme). The schematic in FIG. 3C shows that in cells co-expressing a transcriptionally active form of Cas9 protein, Csy4 ribonuclease, and CMVp-HH-g1-HDV, the guide RNA is expressed.

FIG. 3D shows a graph comparing relative EYFP and mKate2 expression levels from cells co-expressing CMVp-mK-Tr-HH-g1-HDV, CMVp-mK-HH-g1-HDV or CMVp-HH-g1-HDV and P1-EYFP. Expression levels from cells expressing the ‘triplex/Csy4’ construct (mK-Tr-28-g1-28), with and without Csy4, as well as cells expressing the RNAP III promoter, U6p, driving gRNA1 (U6p-g1) are shown for comparison.

FIG. 4A shows an engineered construct that includes a CMV promoter (CMVp) operably linked to a nucleic acid that includes a nucleotide sequence encoding a guide RNA (gRNA1) flanked by Csy4 recognition sites (28 bp), which are flanked by cognate intronic splice sites, which are within a nucleotide sequence encoding an mKate2 protein, which is upstream of a nucleotide sequence encoding a triple helix structure (triplex), which is upstream of a nucleotide sequence encoding a gRNA (gRNA2) flanked by Csy4 recognition sites (28 bp) (Input A, ‘intron-triplex’). Functional gRNA expression was assessed by activation of a gRNA1-specific P1-EYFP construct and a gRNA2-specific P2-ECFP construct.

FIG. 4B shows an engineered construct that includes a CMV promoter (CMVp) operably linked to a nucleic acid that includes a nucleotide sequence encoding a mKate2 protein, which is upstream of a nucleotide sequence encoding a triple helix structure (triplex), which is upstream of a nucleotide sequence encoding two gRNAs (gRNA1 and gRNA2), each flanked by Csy4 recognition sites. The gRNAs are encoded in tandem with intervening and flanking Csy4 recognition sites (Input B, ‘triplex-tandem’). Functional gRNA expression was assessed by activation of a gRNA1-specific P1-EYFP construct and a gRNA2-specific P2-ECFP construct.

FIG. 4C shows a graph demonstrating that both multiplexed gRNA expression constructs (Input A and Input B) exhibited efficient activation of EYFP and ECFP expression in the presence of Csy4, thus demonstrating the generation of multiple active gRNAs from a single transcript. Furthermore, as expected from FIG. 1 and FIG. 2, mKate2 levels decreased with Input A due to the intronic configuration whereas mKate2 levels increased with Input B due to the non-intronic configuration.

FIG. 5A shows an engineered construct that includes a CMV promoter (CMVp) operably linked to a nucleic acid that includes a nucleotide sequence encoding a mKate2 protein, which is upstream of a nucleotide sequence encoding a triple helix structure (triplex), which is upstream of a nucleotide sequence encoding four different gRNAs (gRNAs 3-6), each flanked by Csy4 recognition sites. The gRNAs are encoded in tandem with intervening and flanking Csy4 recognition sites (mK-Tr-(28-g-28)₃₋₆).

FIG. 5B shows a graph demonstrating that the multiplexed mK-Tr-(28-g-28)₃₋₆ construct exhibited high-level activation of IL1RN expression in the presence of Csy4 compared to the same construct in the absence of Csy4. Relative IL1RN mRNA expression was determined compared to a control construct with non-specific gRNA1 (NS, CMVp-mK-Tr-28-g1-28) expressed via the ‘triplex/Csy4’ configuration. For comparison, a non-multiplexed set of plasmids containing the same gRNAs (gRNA3-6), each expressed from separate, individual plasmids is shown.

FIG. 6A shows a three-stage transcriptional cascade implemented by using intronic gRNA1 (CMVp-mKEX1-[28-g1-28]HSV-mKEX2) as the first stage. gRNA1 specifically targeted the P1 promoter to express gRNA2 (P1-EYFP-Tr-28-g2-28), which then activated expression of ECFP from the P2 promoter (P2-ECFP).

FIG. 6B shows a three-stage transcriptional cascade implemented by using a ‘triplex/Csy4’ configuration to express gRNA1 (CMVp-mK-Tr-28-g1-28). gRNA1 specifically targeted the P1 promoter to express gRNA2 (P1-EYFP-Tr-28-g2-28), which then activated expression of ECFP from P2 (P2-ECFP).

FIG. 6C shows a graph demonstrating that the complete three-stage transcriptional cascade from FIG. 6A exhibited expression of all three fluorescent proteins. The removal of one of each of the three stages in the cascade resulted in the loss of fluorescence of the specific stage and dependent downstream stages.

FIG. 6D shows a graph demonstrating that the complete three-stage transcriptional cascade from FIG. 6B exhibited expression of all three fluorescent proteins. The removal of one of each of the three stages in the cascade resulted in the loss of fluorescence of the specific stage and dependent downstream stages.

FIG. 7A shows an engineered construct that encodes both miRNA and CRISPR-TF-based regulation by expressing a miRNA from an intron within mKate2 and gRNA1 from a ‘triplex/Csy4’ configuration (CMVp-mKEx1-[miR]-mKEx2-Tr-28-g1-28). In the presence of taCas9, but in the absence of Csy4, this circuit did not activate a downstream gRNA1-specific P1-EYFP construct and did repress a downstream ECFP transcript with eight (8×) miRNA binding sites flanked by Csy4 recognition sites (CMVp-ECFP-Tr-28-miR8×BS). In the presence of both taCas9 and Csy4, this circuit was rewired by activating gRNA1 production and subsequent EYFP expression as well as by separating the ECFP transcript from the 8× miRNA binding sites, thus ablating miRNA inhibition of ECFP expression.

FIG. 7B shows a graph demonstrating that Csy4 expression can change the behavior of the circuit in FIG. 7A by rewiring circuit interconnections.

FIG. 7C shows a circuit motif diagram illustrating the Csy4-catalyzed rewiring.

FIG. 7D shows an autoregulatory feedback loop incorporated into the network topology of the circuit described in FIG. 7A by encoding 4× miRNA binding sites at the 3′ end of the input transcript (CMVp-mKEx1-[miR]-mKEx2-Tr-28-g1-28-miR4×BS). This negative feedback suppressed mKate2 expression in the absence of Csy4. However, in the presence of Csy4, the 4×miRNA binding sites were separated from the mKate2 mRNA, thus leading to mKate2 expression.

FIG. 7E shows a graph demonstrating that Csy4 expression can change the behavior of the circuit in FIG. 7D by rewiring circuit interconnections. In contrast to the circuit in FIG. 7A, mKate2 was suppressed in the absence of Csy4 but was highly expressed in the presence of Csy4 due to elimination of the miRNA-based autoregulatory negative feedback.

FIG. 7F shows a circuit motif diagram illustrating Csy4-catalyzed rewiring. Each of the mKate2, EYFP, and ECFP levels in FIG. 7B and FIG. 7E were normalized to the respective maximal fluorescence levels amongst all the tested scenarios. The controls in column 3 and 4 in FIGS. 7B and 7E are duplicated, as the two circuits in FIGS. 7A and 7D were tested in the same experiment with the same controls.

FIG. 8A shows flow cytometry data corresponding to the ‘triplex/csy4’ configuration for generating functional gRNAs from RNAP II transcripts.

FIG. 8B shows the ‘intron/Csy4’ configuration for generating functional gRNAs from RNAP II transcripts. Abbreviations: Comp-PE-Tx-Red-YG-A (mKate2); Comp-FITC-A (EYFP). Triplex: construct #3 (CMVp-mK-Tr-28-g1-28, 1 μg). Consensus, snoRNA2, and HSV1: constructs #8-10, respectively (CMVp-mKEX1-[28-g1-28]′intron type′-mKEX2 with the corresponding intron sequences flanking the gRNA and Csy4 recognition sites (‘28’)). These plasmids were transfected at 1 μg. In addition, the amount of the Csy4-expressing plasmid (construct #2) transfected in each sample is indicated. Other plasmids transfected included construct #1 (taCas9, 1 μg) and #5 (P1-EYFP, 1 μg).

FIG. 9 shows flow cytometry data corresponding to FIG. 1B to analyze how various combinations of Csy4 and taCas9 affect expression of the mKate2 gene for the CMVp-mK-Tr-28-g1-28 configuration. Abbreviations: Comp-PE-Tx-Red-YG-A (mKate2). All samples contained Construct #3 (CMVp-mK-Tr-28-g1-28, 1 μg). Construct #1 (taCas9, 1 μg) and Construct #2 (Csy4, 100 ng) were applied as indicated.

FIG. 10 shows flow cytometry data providing various controls to demonstrate minimal non-specific activation of the P1 promoter by gRNA3 (top two panels) and minimal EYFP activation from the promoter P1 with intronic gRNA1 without Csy4 binding sites (bottom panel). Abbreviations: Comp-PE-Tx-Red-YG-A (mKate2); Comp-FITC-A (EYFP). The amount of Csy4 DNA transfected in each sample in the top two panels is indicated in the figure. The lower panel (CMVp-mKEX1-[g1]cons-mKEX2) was tested in the absence of Csy4. Other plasmids transfected in this experiment included construct #1 (taCas9, 1 μg) and construct #5 (P1-EYFP, 1 μg).

FIG. 11 shows flow cytometry data corresponding to FIGS. 2E and 2F to analyze how various configurations of Csy4 recognition sites flanking the gRNA within an intron affect CRISPR-TF activity. Abbreviations: Comp-PE-Tx-Red-YG-A (mKate2); Comp-FITC-A (EYFP). ‘28-gRNA-28’ is HSV1 intronic gRNA flanked by two Csy4 recognition sites (construct #4, CMVp-mKEX1-[28-g1-28]HSV1-mKEX2); ‘28-gRNA’ is HSV1 intronic gRNA with a 5′ Csy4 recognition site only (construct #10, CMVp-mKEX1-[28-g1]HSV1-mKEX2); ‘gRNA-28’ is HSV1 intronic gRNA with a 3′ Csy4 recognition site only (construct #11, CMVp-mKEX1-[g1-28]HSV1-mKEX2). In addition, the amount of the Csy4-expressing plasmid transfected in each sample is indicated with each figure. Other plasmids transfected in this experiment include construct #1 (taCas9, 1 μg) and construct #5 (P1-EYFP 1 μg).

FIG. 12 shows flow cytometry data corresponding to FIG. 3. Abbreviations: Comp-PE-Tx-Red-YG-A (mKate2); Comp-FITC-A (EYFP). ‘Triplex-Csy4’ mechanism contains construct #3 (CMVp-mK-Tr-28-g1-28). Other plasmids transfected in this experiment include construct #1 (taCas9, 1 μg); construct #5 (P1-EYFP); construct #2 (Csy4, concentrations indicated). ‘Ribozyme design 1’ contains construct #13 (CMVp-mK-Tr-HH-g1-HDV). Other plasmids transfected in this experiment include construct #1 (taCas9, 1 μg); construct #5 (P1-EYFP, 1 μg). ‘Ribozyme design 2’ contains construct #14 (CMVp-mK-HH-g1-HD). Other plasmids transfected in this experiment include construct #1 (taCas9, 1 μg); construct #5 (P1-EYFP, 1 μg). ‘Ribozyme design 3’ contains construct #15 (CMVp-HH-g1-HDV). Other plasmids transfected in this experiment include construct #1 (taCas9, 1 μg); construct #5 (P1-EYFP, 1 μg). ‘U6p-gRNA1’ contains construct #7 (U6p-g1, 1 μg). Other plasmids transfected in this experiment include construct #1 (taCas9, 1 μg).

FIG. 13 shows flow cytometry data corresponding to FIG. 4C. Abbreviations: Comp-PE-Tx-Red-YG-A (mKate2); Comp-FITC-A (EYFP); Comp-Pacific Blue-A (ECFP). ‘Mechanism 1’ refers to the ‘intron-triplex’ configuration and contains constructs #16 (CMVp-mKEX1-[28-g1-28]HSV1-mKEX2-Tr-28-g2-28, 1 μg); #5 (P1-EYFP, 1 μg); #6 (P2-ECFP, 1 μg); and #1 (taCas9, 1 μg). ‘Mechanism 2’ refers to the ‘tandem-triplex’ configuration and contains constructs #17 (CMVp-mK-Tr-28-g1-28-g2-28, 1 μg); #5 (P1-EYFP, 1 μg) and #6 (P2-ECFP, 1 μg); and #1 (taCas9, 1 μg). In addition, the amount of Csy4-expressing plasmid DNA (Construct #2) transfected in each sample is indicated above each plot.

FIG. 14 shows flow cytometry data corresponding to FIGS. 6C and 6D. Abbreviations: Comp-PE-Tx-Red-YG-A (mKate2); Comp-FITC-A (EYFP); Comp-Pacific Blue-A (ECFP). All samples were transfected with the constructs listed in each plot title (1 μg each, Table 2) and 200 ng of the Csy4-expressing plasmid (construct #2).

FIG. 15 shows flow cytometry data corresponding to FIGS. 7B and 7E. Abbreviations: Comp-PE-Tx-Red-YG-A (mKate2); Comp-FITC-A (EYFP); Comp-Pacific Blue-A (ECFP). ‘Mechanism 1’ contains the following constructs: #20 (CMVp-mKEx1-[miR]-mKEx2-Tr-28-g1-28); #22 (CMVp-ECFP-Tr-28-miR8×BS-28); and #5 (P1-EYFP). These plasmids were transfected at a concentration of 1 μg each. This mechanism corresponds to the circuit diagram in FIG. 7A. ‘Mechanism 2’ contains the following constructs: #21 (CMVp-mKEx1-[miR]-mKEx2-Tr-28-g1-28-miR4×BS); #22 (CMVp-ECFP-Tr-28-miR8×BS-28); and #5 (P1-EYFP). These plasmids were transfected at a concentration of 1 μg each. This mechanism corresponds to the circuit diagram in FIG. 7D. ‘Control’ samples contain constructs #22 (CMVp-ECFP-Tr-28-miR8×BS-28) and #5 (P1-EYFP) only. These plasmids were transfected at a concentration of 1 μg each. In addition, the amount of Csy4-expressing plasmid (Construct #2) transfected in each sample is indicated above each plot.

### DETAILED DESCRIPTION OF INVENTION

The ability to build complex, robust and scalable synthetic gene networks that operate with defined interconnections between artificial parts and native cellular processes is central to engineering biological systems. This capability can enable new strategies, for example, for rewiring, perturbing and probing natural biological networks. A large set of tunable, orthogonal, compact and multiplexable gene regulatory mechanisms is of fundamental importance to implement these applications. Despite much progress in the fields of transcriptional regulation and synthetic biology, the tools that were available prior to the present disclosure fail to meet one or more of the criteria described above. Transcriptional regulation utilizes transcription factors that bind predetermined DNA sequences of interest. Type II CRISPR/Cas systems (e.g., with DNA-targeting Cas proteins) have been adapted to achieve programmable DNA binding without requiring complex protein engineering (Sander and Joung, 2014). In these systems, the sequence specificity of the Cas9 DNA-binding protein is determined by guide RNAs (gRNAs), which have base-pairing complementarity to target DNA sites. This enables simple and highly flexible programming of Cas9 binding.

Prior to the present disclosure, gRNAs for gene regulation in human cells were expressed only from RNA polymerase III (RNAP III) promoters. This is a limitation in terms of integrating CRISPR/Cas regulation with endogenous gene networks because RNAP III promoters comprise only a small portion of cellular promoters and are mostly constitutively active, thus preventing the linkage of most cellular promoters and signals into CRISPR-TF-based networks. Further, multiple gRNAs are typically needed to efficiently activate endogenous promoters, but strategies for multiplexed gRNA production from single transcripts for transcriptional regulation were not available prior to the present disclosure. As a result, multiple gRNA expression constructs were needed to perturb natural transcriptional networks, thus limiting scalability.

In addition to transcriptional regulation, natural circuits leverage RNA-based translational and post-translational regulation to achieve complex behavior. Synthetic gene regulatory strategies that combine RNA and transcriptional engineering, as provided herein, are useful in modeling natural systems or implementing artificial behaviors. Thus provided herein, in various aspects, are methods and compositions that integrate mammalian and bacterial RNA-based regulatory mechanisms to, for example, create complex synthetic circuit topologies and to regulate endogenous promoters. Multiple mammalian RNA processing strategies can be used, including 3′ RNA triple helixes (referred to as triplexes), introns and ribozymes, together with mammalian miRNA regulation, bacteria-derived CRISPR-TFs and the Csy4 RNA-modifying protein from P. aeruginosa. These constructs can be used, for example, to generate functional gRNAs from RNAP-II-regulated mRNAs in human cells while rendering the concomitant translation of the mRNAs tunable.

As shown herein, functional gRNAs were used to target both synthetic and endogenous promoters for activation via CRISPR-TFs. Additionally, strategies for multiplexed gRNA production were developed, thus enabling compact encoding of proteins and multiple gRNAs in single transcripts. To demonstrate the utility of these regulatory parts, multi-stage transcriptional cascades that can be used for the construction of complex synthetic gene circuits were implemented. Also combined herein are mammalian miRNA-based regulation with CRISPR-TFs to create multicomponent genetic circuits with feedback loops, interconnections, and behaviors that can be rewired, in some embodiments, by Csy4-based RNA processing. Thus, the platform of the present disclosure can be used, for example, to construct, synchronize and switch complex regulatory networks, both artificial and endogenous, using synthetic transcriptional and RNA-dependent mechanisms. The integration of CRISPR-TF-based gene regulation systems with mammalian RNA regulatory configurations, in some embodiments, enables scalable gene regulatory systems for synthetic biology as well as basic biology applications.

Aspects of the present disclosure relate to engineered constructs and engineered nucleic acids. “Engineered construct” is a term used to describe an engineered nucleic acid having multiple genetic elements, including, for example, a promoter and various nucleotide sequences (e.g., nucleotide sequences encoding a protein and/or an RNA interference molecule, as provided herein). A nucleic acid is at least two nucleotides covalently linked together, and in some instances, may contain phosphodiester bonds (e.g., a phosphodiester “backbone”). An engineered nucleic acid is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) and, in some embodiments, can replicate in a living cell. A synthetic nucleic acid is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing.

In some embodiments, a nucleic acid of the present disclosure is considered to be a nucleic acid analog, which may contain, at least in part, other backbones comprising, for example, phosphoramide, phosphorothioate, phosphorodithioate, O-methylphophoroamidite linkages and/or peptide nucleic acids. A nucleic acid may be single-stranded (ss) or double-stranded (ds), as specified, or may contain portions of both single-stranded and double-stranded sequence. In some embodiments, a nucleic acid may contain portions of triple-stranded sequence. A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine.

Engineered constructs (including engineered nucleic acids) of the present disclosure include one or more genetic elements. A “genetic element” refers to a particular nucleotide sequence that has a role in nucleic acid expression (e.g., promoter, enhancer, terminator) or encodes a discrete product of an engineered nucleic acid (e.g., a nucleotide sequence encoding a guide RNA, a protein and/or an RNA interference molecule). Examples of genetic elements of the present disclosure include, without limitation, promoters and nucleotide sequences that encode proteins, guide RNAs, Csy4 binding sites, triple helix structures, introns and intronic sequences (e.g., donor site, acceptor site and/or branch site), exons and ribozymes.

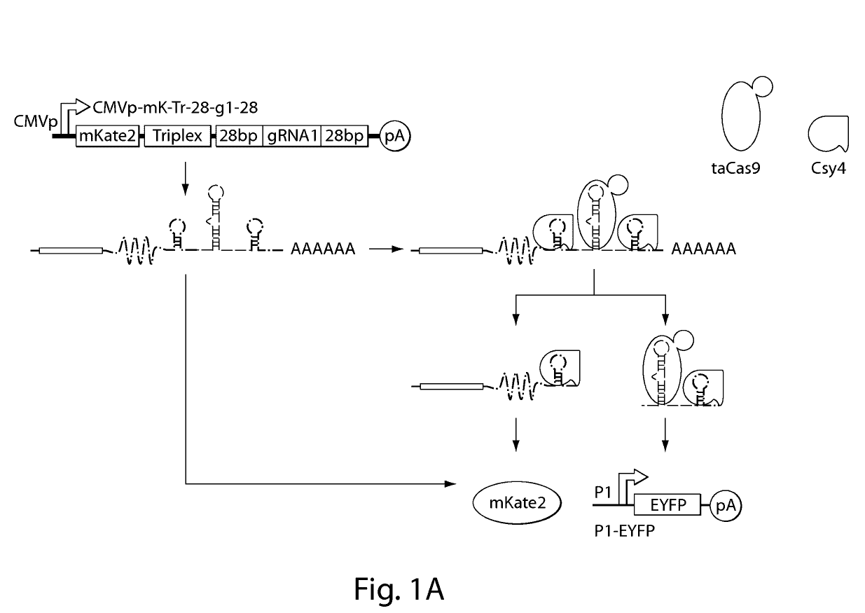

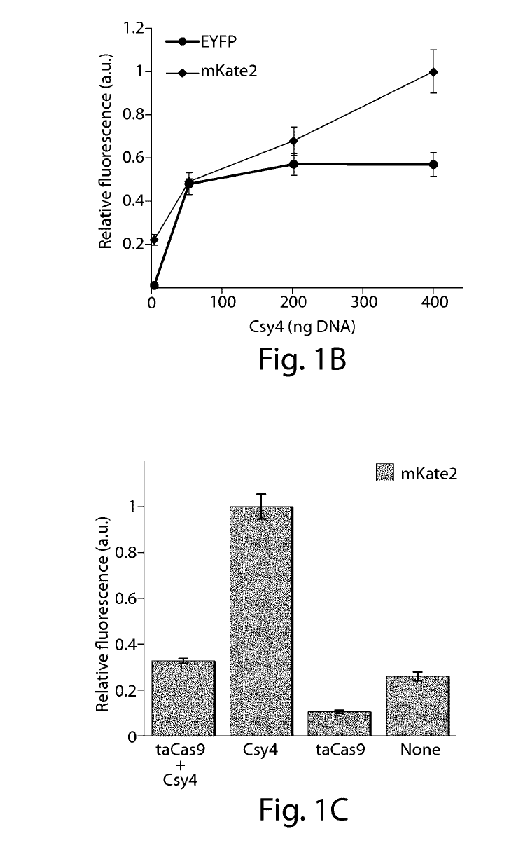

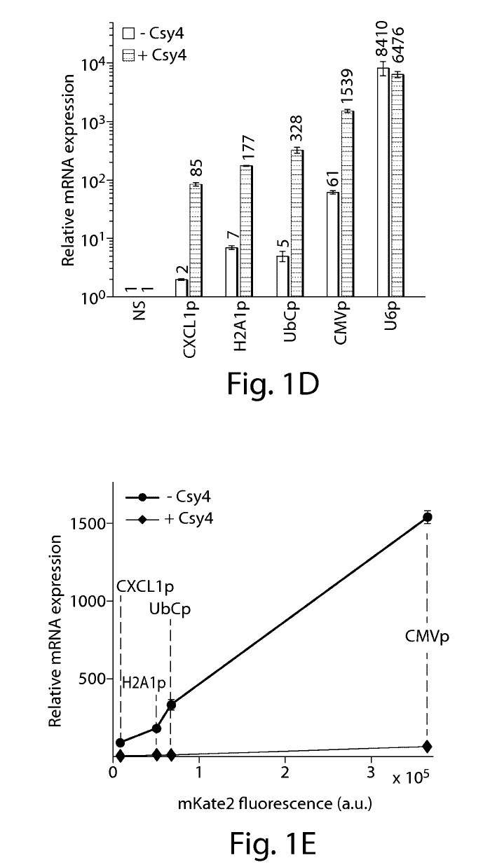

The position of a genetic element of an engineered nucleic acid of the present disclosure may be defined relative to other genetic elements along a 5′ to 3′ oriented coding (sense) strand. For example, FIG. 1A shows a CMV promoter operably linked to a nucleotide sequence encoding an mKate2 protein, which is upstream of a nucleotide sequence encoding a triple helix structure (or “triplex”), which is upstream of a nucleotide sequence encoding a guide RNA flanked by Csy4 binding sites. Alternatively, the engineered nucleic acid depicted in FIG. 1A may be described as having a nucleotide sequence encoding a guide RNA flanked by Csy4 binding sites, which is downstream of a nucleotide sequence encoding a triple helix structure, which is downstream of a nucleotide sequence encoding an mKate2 protein, which is operably linked to an upstream promoter. Thus, a first genetic element is considered to be downstream of a second genetic element if the first genetic element is located 3′ of the second genetic element. Likewise, a second genetic element is considered to be upstream of a first genetic element if the second genetic element is located 5′ of the first genetic element. One genetic element is considered to be “immediately downstream” or “immediately upstream” of another genetic element if the two genetic elements are proximal to each other (e.g., no other genetic element is located between the two). In the configuration shown in FIG. 1A, for example, a nucleotide sequence encoding a guide RNA flanked by Csy4 binding sites is immediately downstream of a nucleotide sequence encoding a triple helix structure.

Some aspects of the present disclosure relate to engineered nucleic acids that include a (e.g., one or more, at least one) nucleotide sequence encoding a (e.g., at least one, including at least 2, at least 3, at least 4, at least 5, at least 6, or more) guide RNA (gRNA). A gRNA is a component of the CRISPR/Cas system. CRISPR/Cas systems are used by various bacteria and archaea to mediate defense against viruses and other foreign nucleic acid. Components of the CRISPR/Cas system coordinate to selectively cleave nucleic acid. Type II CRISPR/Cas systems include Cas proteins that are targeted to DNA, while type III CRISPR/Cas systems include Cas proteins that are targeted to RNA. The sequence specificity of a Cas DNA-binding protein is determined by gRNAs, which have base-pairing complementarity to target DNA sites. Thus, Cas proteins are “guided” by gRNAs to target DNA sites. The base-pairing complementarity of gRNAs enables, in some embodiments, simple and flexible programming of Cas binding. Base-pair complementarity refers to distinct interactions between adenine and thymine (DNA) or uracil (RNA), and between guanine and cytosine.

Guide RNAs of the present disclosure, in some embodiments, have a length of 10 to 500 nucleotides. In some embodiments, a gRNA has a length of 10 to 20 nucleotides, 10 to 30 nucleotides, 10 to 40 nucleotides, 10 to 50 nucleotides, 10 to 60 nucleotides, 10 to 70 nucleotides, 10 to 80 nucleotides, 10 to 90 nucleotides, 10 to 100 nucleotides, 20 to 30 nucleotides, 20 to 40 nucleotides, 20 to 50 nucleotides, 20 to 60 nucleotides, 20 to 70 nucleotides, 20 to 80 nucleotides, 20 to 90 nucleotides, 20 to 100 nucleotides, 30 to 40 nucleotides, 30 to 50 nucleotides, 30 to 60 nucleotides, 30 to 70 nucleotides, 30 to 80 nucleotides, 30 to 90 nucleotides, 30 to 100 nucleotides, 40 to 50 nucleotides, 40 to 60 nucleotides, 40 to 70 nucleotides, 40 to 80 nucleotides, 40 to 90 nucleotides, 40 to 100 nucleotides, 50 to 60 nucleotides, 50 to 70 nucleotides, 50 to 80 nucleotides, 50 to 90 nucleotides or 50 to 100 nucleotides. In some embodiments, a gRNA has a length of 10 to 200 nucleotides, 10 to 250 nucleotides, 10 to 300 nucleotides, 10 to 350 nucleotides, 10 to 400 nucleotides or 10 to 450 nucleotides. In some embodiments, a gRNA has a length of more than 500 nucleotides. In some embodiments, a gRNA has a length of 10, 15, 20, 15, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 or more nucleotides.

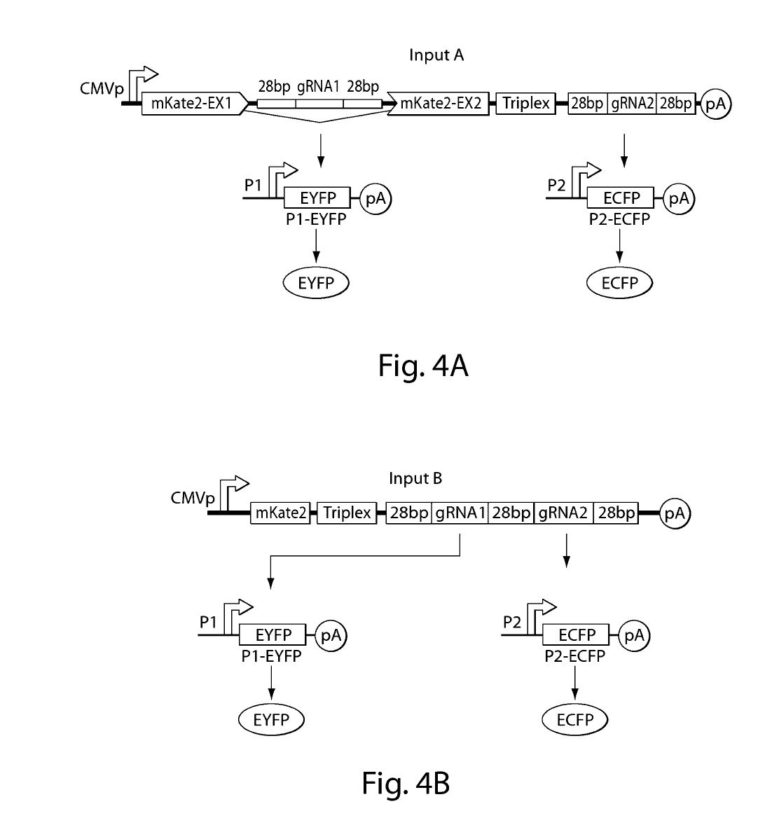

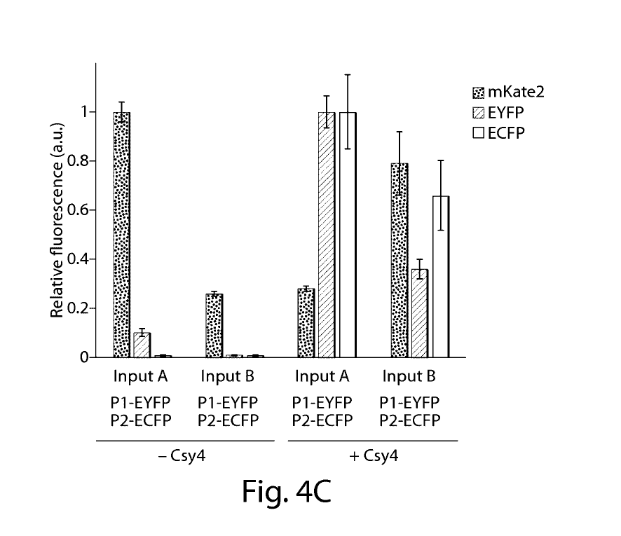

The methods and compositions of the present disclosure, surprisingly, permit production of multiple guide RNAs (gRNAs), in some embodiments, from a single transcript. It should be understood, however, that multiple gRNAs may produced from multiple transcripts in a single cell. gRNAs produced as provided herein may have the same nucleotide sequence or may have different nucleotide sequences. Thus, gRNAs may target and bind to the same target site or different target site (e.g., a region within a particular promoter). For example, some engineered nucleic acids comprise a nucleotide sequence encoding a first gRNA and a nucleotide sequence encoding a second gRNA (or a nucleotide sequence encoding at least two gRNAs). The first gRNA may have the same RNA sequence as the second gRNA, and, thus the two gRNAs may target the same site. Alternatively, the first gRNA may have a RNA sequence that is different from the second gRNA, and, thus, the two gRNAs may target the different sites (e.g., within the same promoter of within different promoters). As exemplified in FIG. 4A, “gRNA1” targets a promoter (P1) operably linked to enhanced yellow fluorescent protein (EYFP), while “gRNA2” targets a promoter (P2) operably linked to enhanced cyan fluorescent protein (ECFP).

A first nucleotide sequence is considered to be “within” a second nucleotide sequence if the first nucleotide sequence is inserted between two nucleotides of the second nucleotide sequence, or if the nucleotide sequence replaces a stretch of contiguous nucleotides of the second nucleotide sequence. In some embodiments, a nucleotide sequence encodes a gRNA or an RNA interference molecule within a protein of interest. In this configuration, a nucleotide sequence encoding a gRNA, for example, is positioned between two adjacent exons of the protein of interest such that when the encoded gRNA is removed (e.g., by RNA splicing if the gRNA is flanked by cognate intronic splice sites) the protein is translated. Guide RNAs, as discussed above, “guide” Cas proteins to a nucleic acid, in some embodiments.

Cas proteins are nucleases that cleave nucleic acid. The nuclease activity of Cas proteins (e.g., Cas9 proteins), in some embodiments, can be utilized for precise and efficient genome editing in prokaryotic and eukaryotic cells. Mutant Cas proteins are also contemplated herein. In some embodiments, a mutant Cas protein lacks nuclease activity (e.g., dCas9). In some embodiments, a mutant Cas protein lacking nuclease activity is modified to enable programmable transcriptional regulation of both ectopic and native promoters to create CRISPR-based transcription factors (CRISPR-TFs) in mammalian cells (Cheng et al., 2013; Farzadfard et al., 2013; Gilbert et al., 2013; Maeder et al., 2013a; Mali et al., 2013a; Perez-Pinera et al., 2013a). For example, fusing an activation domain (e.g., VP16, VP64 or p65) to a Cas protein renders the Cas transcriptionally active (also referred to as a “taCas” protein). Transcriptional activator proteins recruit the RNA polymerase II machinery and chromatin-modifying activities to promoters. Thus, in some embodiments, “transcriptionally active” Cas (taCas) proteins, which lack nuclease activity, are used in accordance with the present disclosure. In some embodiments, a transcriptionally active Cas protein is a transcriptionally active Cas9 (taCas9) protein. Other transcriptionally active Cas proteins are contemplated herein.

In some embodiments, a guide RNA of the present disclosure is flanked by ribonuclease recognition sites. A ribonuclease (abbreviated as RNase) is a nuclease that catalyzes the hydrolysis of RNA. A ribonuclease may be an endoribonuclease or an exoribonuclease. An endoribonuclease cleaves either single-stranded or double-stranded RNA. An exoribonuclease degrades RNA by removing terminal nucleotides from either the 5′ end or the 3′ end of the RNA. In some embodiments, a guide RNA of the present disclosure is flanked by Csy ribonuclease recognition sites (e.g., Csy4 ribonuclease recognition sites). Csy4 is an endoribonuclease that recognizes a particular RNA sequence, cleaves the RNA, and remains bound to the upstream fragment. In some embodiments, a Csy ribonuclease (e.g., Csy4 ribonuclease) is used to release a guide RNA from an engineered nucleic acid transcript. Thus, in some embodiments, cells are co-transfected with an engineered construct that comprises a nucleotide sequence encoding a guide RNA flanked by Csy4 or other Cas6 ribonuclease recognition sites and an engineered nucleic acid encoding a Csy4 or other Cas6 ribonuclease. Alternatively, or in addition, the cell may stably express, or be modified to stably express, a Csy4 or other Cas6 ribonuclease. In some embodiments, a Csy ribonuclease (e.g., Csy4 ribonuclease) is from Pseudomonas aeruginosa, Staphylococcus epidermidis, Pyrococcus furiosus or Sulfolobus solfataricus. Other ribonucleases and ribonuclease recognitions sites are contemplated herein (see, e.g., Mojica, F. J. M. et al., CRISPR-Cas Systems, RNA-mediated Adaptive Immunity in Bacteria and Archaea, Barrangou, Rodolphe, van der Oost, John (Eds.), 2013, ISBN 978-3-642-34657-6, of which the subject matter relating to ribonucleases/recognition sites is incorporated by reference herein).

In some embodiments, a ribonuclease recognition site (e.g., Csy4 ribonuclease recognition site) is 10 to 50 nucleotides in length. For example, a Csy ribonuclease recognition site may be 10 to 40, 10 to 30, 10 to 20, 20 to 50, 20 to 40 or 20 to 30 nucleotides in length. In some embodiments, a Csy ribonuclease recognition site is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In some embodiments, a Csy ribonuclease recognition site (e.g., Csy4 ribonuclease recognition site) is 28 nucleotides in length. In some embodiments, the nucleotide sequence encoding a ribonuclease recognition site comprises SEQ ID NO: 26. Csy homologs are also contemplated herein (see, e.g., Mojica, F. J. M. et al., CRISPR-Cas Systems, RNA-mediated Adaptive Immunity in Bacteria and Archaea, Barrangou, Rodolphe, van der Oost, John (Eds.), 2013, ISBN 978-3-642-34657-6, of which the subject matter relating to ribonucleases/recognition sites is incorporated by reference herein).

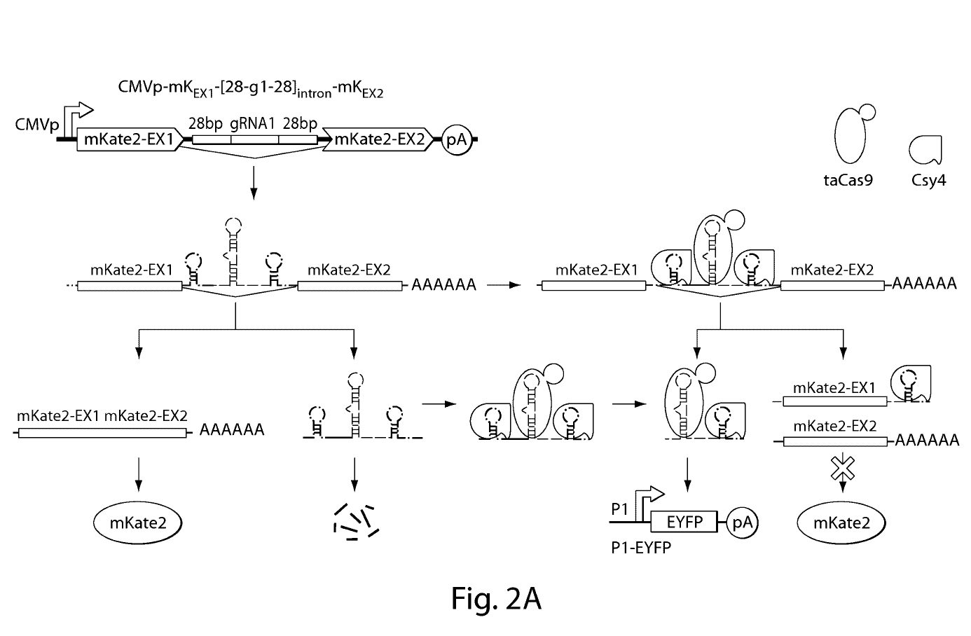

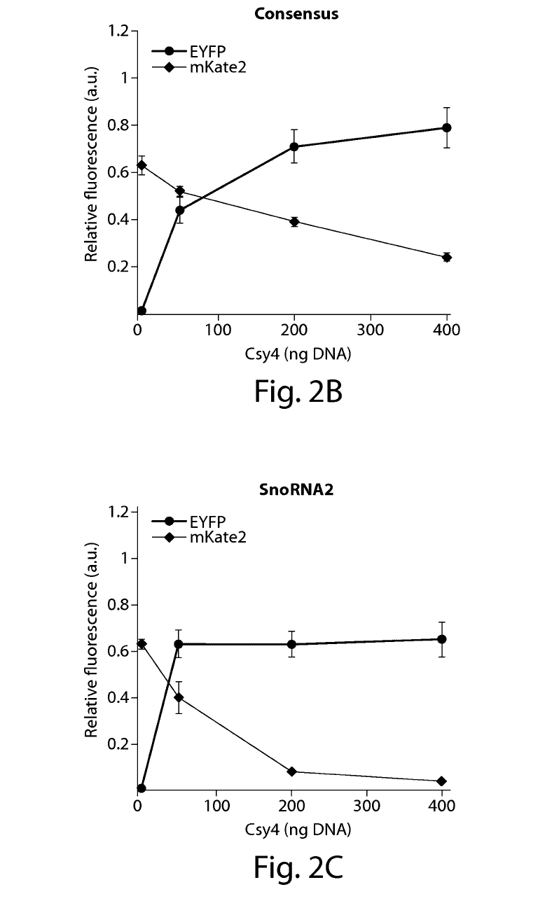

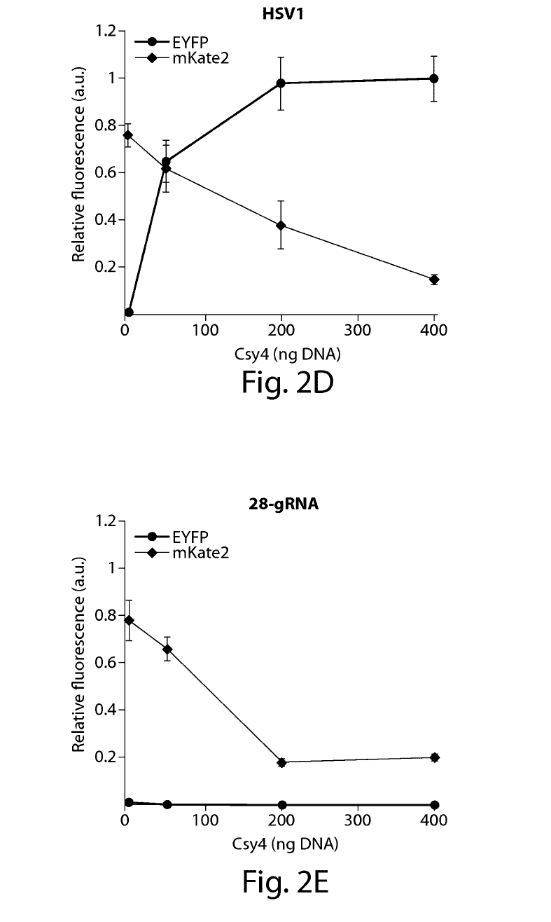

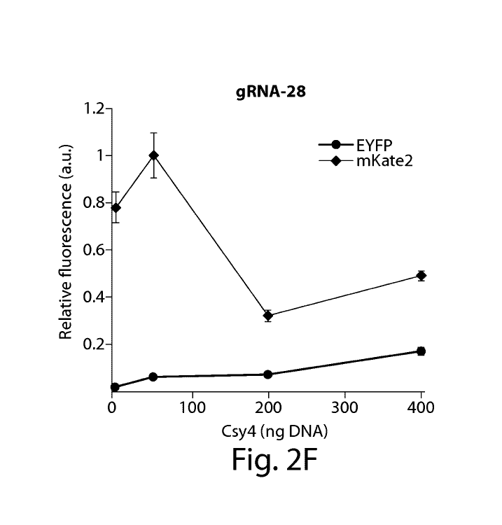

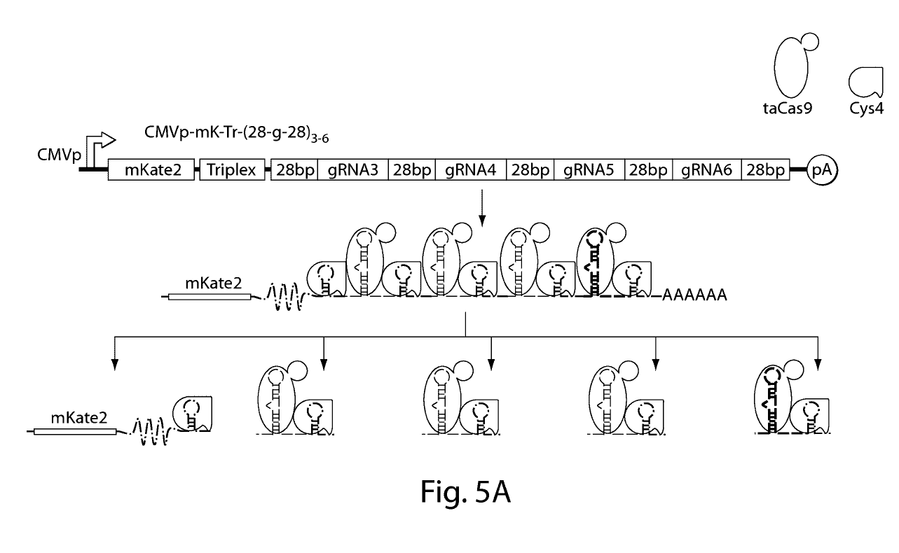

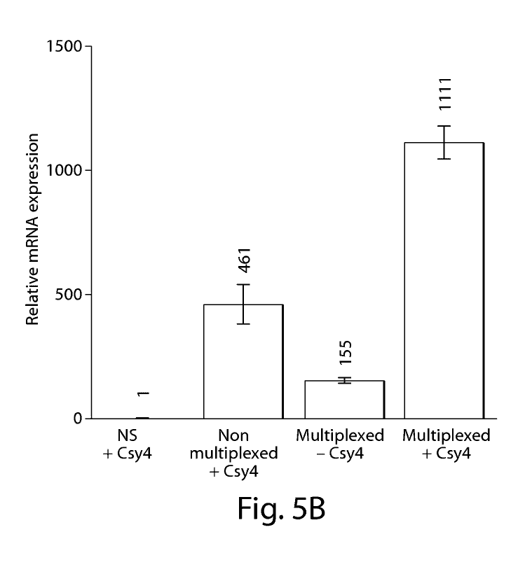

A first genetic element is said to be “flanked” by other genetic elements when the first genetic element is located between and immediately adjacent to the other genetic elements. FIG. 1A, for example, shows a schematic representative of a nucleotide sequence encoding “gRNA1” flanked by Csy4 binding sites (“28 bp”). Similarly, the schematic in FIG. 2A is representative of a nucleotide sequence encoding “gRNA1” flanked by Csy4 binding sites (“28 bp”), which are further flanked by nucleotide sequences encoding cognate intronic splice sites, which are further flanked by nucleotide sequences encoding exons of the mKate2 protein. In some embodiments, engineered constructs contain multiple gRNAs in tandem, as shown in, for example, in FIG. 5A. Such a construct may be described herein as having a nucleotide sequence encoding at least two gRNAs, each gRNA flanked by ribonuclease recognition sites. It should be understood that this configuration is meant to encompass multiple gRNAs in tandem, each gRNA flanked by a single ribonuclease recognition site (RRS), as shown in FIG. 5A (RRS referred to as ‘28 bp’ in the figure), as well as multiple gRNAs in tandem, each gRNA flanked by two or more ribonuclease recognition sites. For example, the genetic elements may be ordered in an engineered construct as follows: RRS1-gRNA1-RRS2-gRNA2-RRS3-gRNA-RRS4 whereby a single ribonuclease recognition site separates one gRNA from an adjacent gRNA; or RRS1-gRNA1-RRS2-RRS3-gRNA2-RRS4-RRS5-gRNA-RRS6, whereby two ribonuclease recognition sites separate one gRNA from an adjacent gRNA. The RRS may be the same or different. That is, different types of ribonucleases may be used, in some embodiments, to release one or more gRNAs from an engineered construct.

Some aspects of the present disclosure relate to engineered constructs that include a 3′ RNA stabilizing sequence such as, for example, an RNA sequence that forms a triple helix structure (or “triplex”). A 3′ RNA stabilizing sequence is a nucleotide sequence added to the 3′ end of a nucleotide sequence encoding a product to complement for the lack of a poly-(A) tail. Thus, 3′ RNA stabilizing sequences, such as those that form triple helix structures, in some embodiments, enable efficient translation of mRNA lacking a poly-(A) tail. A triple helical structure is a secondary or tertiary RNA structure formed, for example, by adenine- and uridine-rich motifs. In some embodiments, a 3′ RNA stabilizing sequence is from a 3′ untranslated region (UTR) of a nucleic acid.

A triple helix structure, in some embodiments, promotes RNA stability and/or translation. In some embodiments, a triple helix structure of the present disclosure is encoded by a nucleotide fragment from the 3′ end of the MALAT1 (metastasis-associated lung adenocarcinoma transcript 1) locus or the MENβ (multiple endocrine neoplasia-β) locus. In some embodiments, a triple helix structure is encoded by a nucleotide fragment from the 3′ end of the MALAT1 locus or the 3′ end of the MENβ locus (see, e.g., Wilusz et al., 2012, incorporated by reference herein; see also, Brown J A et al. Proc Natl Acad Sci USA. 2012 Nov. 20; 109(47), incorporated by reference herein). In some embodiments, a triple helix structure is encoded by a 110 nucleotide sequence (e.g., 110 contiguous nucleotide sequences) from the 3′ end of the MALAT1 locus. In some embodiments, a triple helix structure is encoded by a nucleic acid comprising or consisting of SEQ ID NO: 1. Other 3′ RNA stabilizing sequences, included those that encode triple helix structures, are contemplated herein (see, e.g., Wilusz J. E. et al. RNA 2010. 16: 259-266, incorporated by reference herein).

Some aspects of the present disclosure relate to engineered constructs that include a nucleotide sequence encoding a gRNA flanked by ribonuclease (e.g., Csy4) recognition sites, wherein the nucleotide sequence is flanked by nucleotide sequences encoding cognate intronic splice sites. In the art, the term “intron” often refers to both the DNA sequence within a gene and the corresponding sequence in an RNA transcript. For clarity and consistency herein, it should be understood that in the context of an engineered construct, “a nucleotide sequence encoding an intron” refers to a DNA sequence, while the term “intron” refers to an RNA sequence. An intron is a non-coding RNA sequence that is removed by RNA splicing. RNA splicing is the process by which pre-messenger RNA is modified to remove introns and bring together exons (e.g., protein-coding region of a nucleic acid) to form a mature messenger RNA (mRNA) molecule. “Cognate intronic splice sites” include a donor site (e.g., at the 5′ end of an intron), a branch site (e.g., near the 3′ end of the intron) and an acceptor site (e.g., at the 3′ end of the intron) such that during RNA splicing any intervening sequence (e.g., sequence between the 5′ splice site and the 3′ splice site) is removed. For example, the engineered construct depicted in FIG. 2A includes an intervening genetic element (e.g., a nucleotide sequence encoding a gRNA flanked by Csy4 binding sites) flanked by intronic splice sites. During processing of the transcript produced from the engineered construct of FIG. 2A, the intervening genetic element is removed.

In some embodiments, a 5′ splice donor site includes an almost invariant sequence GU within a larger, less highly conserved region. In some embodiments, a 3′ splice acceptor site includes an almost invariant AG sequence. In some embodiments, upstream of the AG there is a region high in pyrimidines (e.g., C and U), referred to as a polypyrimidine tract. Upstream of the polypyrimidine tract, in some embodiments, is a branchpoint, which may include, for example, an adenine nucleotide. In some embodiments, the consensus sequence for an intron (in IUPAC nucleic acid notation) is: M-A-G-[cut]-G-U-R-A-G-U (donor site) . . . intron sequence . . . C-U-R-[A]Y (branch sequence, e.g., 20-50 nucleotides upstream of acceptor site) . . . Y-rich-N-C-A-G-[cut]-G (acceptor site).

Contemplated herein, in some embodiments, are intronic sequences that produce relatively stable (e.g., “long-lived”) introns. Examples of such sequences include, without limitation, the HSV-1 latency associated intron, which forms a stable circular intron (Block and Hill, 1997), and the sno-IncRNA2 intron (Yin et al., 2012). The sno-IncRNA2 intron (or “sno-RNA2 intron) is processed on both ends by the snoRNA machinery, which protects it from degradation and leads to the accumulation of IncRNAs flanked by snoRNA sequences, which lack 5′ caps and 3′ poly-(A) tails. Other sequences that confer structural stability to an intronic sequence are also contemplated herein.

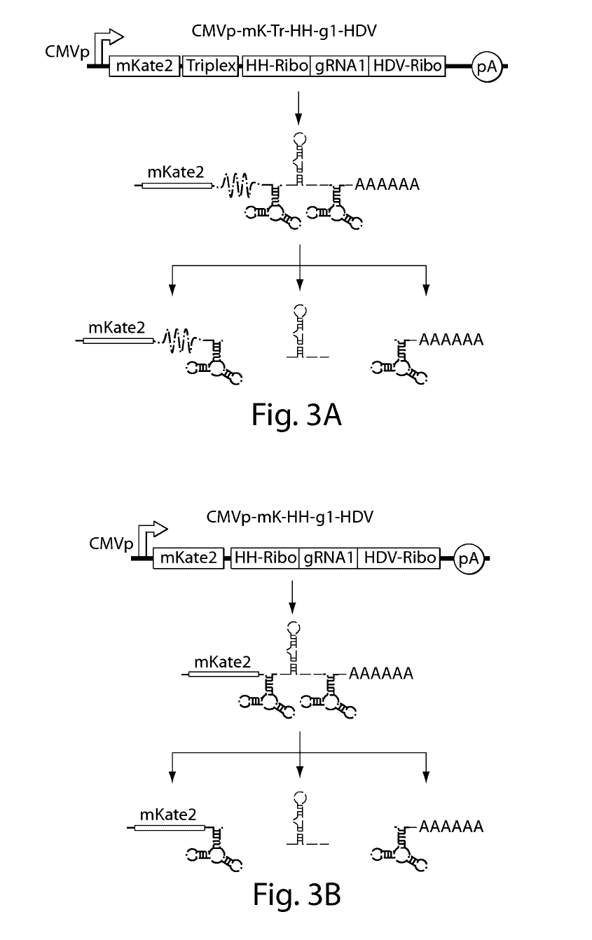

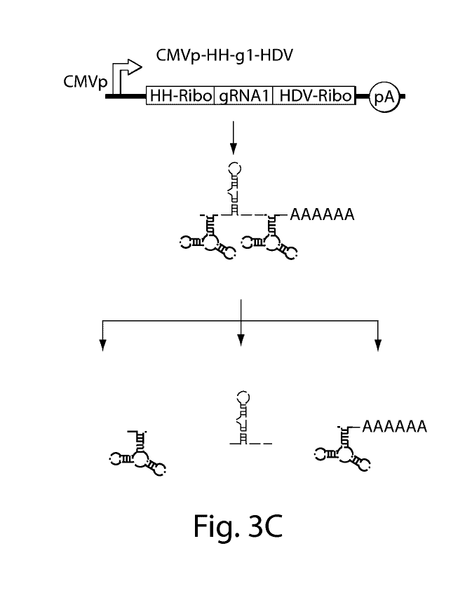

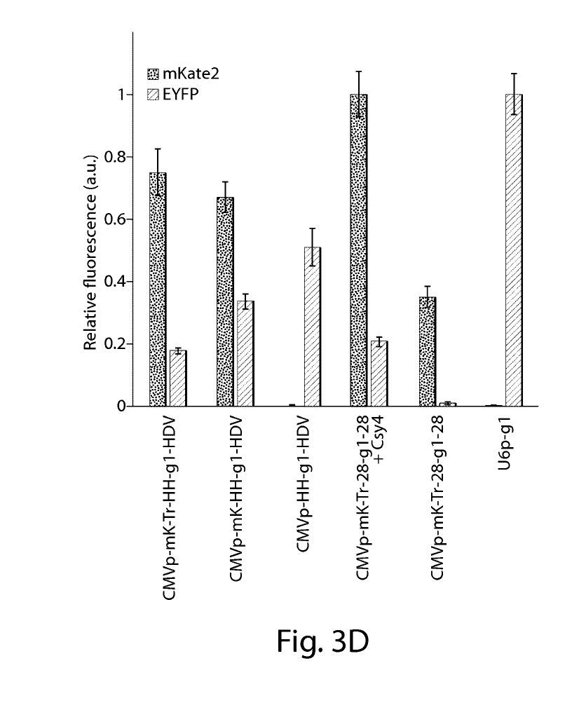

Some aspects of the present disclosure relate to engineered constructs that include a nucleotide sequence encoding a gRNA flanked by ribozymes. Ribozymes are RNA molecules that are capable of catalyzing specific biochemical reactions, similar to the action of protein enzymes. Cis-acting ribozymes are typically self-forming and capable of self-cleaving. Cis-acting ribozymes can mediate functional gRNA expression from RNA pol II promoters. Trans-acting ribozymes, by comparison, do not perform self-cleavage. Self-cleavage refers to the process of intramolecular catalysis in which the RNA molecule containing the ribozyme is itself cleaved. Examples of cis-acting ribozymes for use in accordance with the present disclosure include, without limitation, hammerhead (HH) ribozyme (see, e.g., Pley et al., 1994, incorporated by reference herein) and Hepatitis delta virus (HDV) ribozyme (see, e.g., Ferre-D'Amare et al., 1998, incorporated by reference herein). Examples of trans-acting ribozymes for use in accordance with the present disclosure include, without limitation, natural and artificial versions of the hairpin ribozymes found in the satellite RNA of tobacco ringspot virus (sTRSV), chicory yellow mottle virus (sCYMV) and arabis mosaic virus (sARMV). FIGS. 3A-3C, for example, shows schematics representative of a nucleotide sequence encoding “gRNA1” flanked by ribozymes. In some embodiments, engineered constructs contain multiple gRNAs in tandem, each flanked by nucleotide sequences encoding ribozymes. Such a construct may be described herein as having a nucleotide sequence encoding at least two gRNAs, each gRNA flanked by ribozymes. It should be understood that this configuration is meant to encompass multiple gRNAs in tandem, each gRNA flanked by a single ribozyme (Ribo), as well as multiple gRNAs in tandem, each gRNA flanked by two or more ribozymes. For example, the genetic elements may be ordered in an engineered construct as follows: Ribo1-gRNA1-Ribo2-gRNA2-Ribo3-gRNA-Ribo4 whereby a single ribozyme separates one gRNA from an adjacent gRNA; or Ribo1-gRNA1-Ribo2-Ribo3-gRNA2-Ribo4-Ribo5-gRNA-Ribo6, whereby two ribozymes separate one gRNA from an adjacent gRNA. The ribozymes may be the same or different. That is, different types of ribozymes may be used, in some embodiments, to release one or more gRNAs from an engineered construct.

Some aspects of the present disclosure relate to nucleic acids encoding proteins of interest. A protein of interest may be any protein. Examples of proteins of interest include, without limitation, those involved in cell signaling (e.g., receptor/ligand binding) and signal transduction. A protein of interest may be, for example, a fibrous protein or a globular protein. Examples of fibrous proteins include, without limitation, cytoskeletal proteins and extracellular matrix proteins. Examples of globular proteins include, without limitation, plasma proteins (e.g., coagulation factors, acute phase proteins), hemoproteins, cell adhesion proteins, transmembrane transport proteins (e.g., ion channel proteins, synport proteins, antiport proteins), hormones and growth factors, receptors (e.g., transmembrane receptors, intracellular receptors), DNA-binding proteins (e.g., transcription factors or other proteins involved in transcriptional regulation), immune system proteins, nutrient storage/transport proteins, chaperone proteins, and enzymes. Other proteins are contemplated and may be used in accordance with the present disclosure.

Some aspects of the present disclosure contemplate integrating CRISPR-based mechanisms with mammalian RNA interference mechanisms to, for example, implement more sophisticated circuit topologies. As shown in non-limiting Example 8, micro RNA regulation was incorporated with CRISPR-TFs and Csy4 to disrupt miRNA inhibition of target RNAs by removing cognate miRNA binding sites. RNA interference generally refers to a biological process in which RNA molecules inhibit gene expression, typically by causing the destruction of specific mRNA molecules. Examples of such RNA molecules include microRNA (miRNA) and small interfering RNA (siRNA).

miRNAs are short, non-coding, single-stranded RNA molecules. miRNAs of the present disclosure may be naturally-occurring or synthetic (e.g., artificial). miRNAs usually induce gene silencing by binding to target sites found within the 3′ UTR (untranslated region) of a targeted mRNA. This interaction prevents protein production by suppressing protein synthesis and/or by initiating mRNA degradation. Most target sites on the mRNA have only partial base complementarity with their corresponding microRNA, thus, individual microRNAs may target 100 different mRNAs, or more. Further, individual mRNAs may contain multiple binding sites for different miRNAs, resulting in a complex regulatory network. In some embodiments, a miRNA is 10 to 50 nucleotides in length. For example, a miRNA may be 10 to 40, 10 to 30, 10 to 20, 20 to 50, 20 to 40 or 20 to 30 nucleotides in length. In some embodiments, a miRNA is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In some embodiments, a miRNA is 22 nucleotides in length.

siRNAs are short, non-coding, single-stranded RNA molecules. siRNAs of the present disclosure may be naturally-occurring or synthetic (e.g., artificial). Binding of a siRNA to a cognate mRNA typically results in degradation of the mRNA. In some embodiments, a siRNA is 10 to 50 nucleotides in length. For example, a siRNA may be 10 to 40, 10 to 30, 10 to 20, 20 to 50, 20 to 40 or 20 to 30 nucleotides in length. In some embodiments, a siRNA is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In some embodiments, a siRNA is 21 to 25 nucleotides in length. Engineered constructs of the present disclosure comprise, in some embodiments, promoters operably linked to a nucleotide sequence (e.g., encoding a protein of interest). A “promoter” is a control region of a nucleic acid at which initiation and rate of transcription of the remainder of a nucleic acid are controlled. A promoter may also contain sub-regions at which regulatory proteins and molecules may bind, such as RNA polymerase and other transcription factors. Promoters may be constitutive, inducible, activatable, repressible, tissue-specific or any combination thereof.

A promoter drives expression or drives transcription of the nucleic acid sequence that it regulates. A promoter is considered to be “operably linked” when it is in a correct functional location and orientation in relation to the nucleotide sequence it regulates to control (“drive”) transcriptional initiation and/or expression of that sequence.

A promoter may be classified as strong or weak according to its affinity for RNA polymerase (and/or sigma factor); this is related to how closely the promoter sequence resembles the ideal consensus sequence for the polymerase. The strength of a promoter may depend on whether initiation of transcription occurs at that promoter with high or low frequency. Different promoters with different strengths may be used to construct nucleic acids with different levels of gene/protein expression (e.g., the level of expression initiated from a weak promoter is lower than the level of expression initiated from a strong promoter).

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, gRNAs of the present disclosure are designed to target endogenous promoters (e.g., endogenous human promoter).

In some embodiments, nucleotide sequence may be positioned under the control of a recombinant or heterologous promoter, which refers to a promoter that is not normally associated with the nucleotide sequence in its natural environment. Such promoters may include promoters of other genes; promoters isolated from any other prokaryotic cell; and synthetic promoters that are not “naturally occurring” such as, for example, those that contain different elements of different transcriptional regulatory regions and/or mutations that alter expression through methods of genetic engineering that are known in the art. In addition to producing nucleotide sequences of promoters synthetically, sequences may be produced using recombinant cloning and/or nucleic acid amplification technology, including polymerase chain reaction (PCR).

In some embodiments, initiation of transcription from a promoter depends on the activity of RNA polymerase (also referred to as DNA-dependent RNA polymerase). RNA polymerases are nucleotidyl transferase that polymerizes ribonucleotides at the 3′ end of an RNA transcript. Eukaryotes have multiple types of nuclear RNA polymerases, each responsible for synthesis of a distinct subset of RNA. All are structurally and mechanistically related to each other and to bacterial RNA polymerase. RNA polymerase I synthesizes a pre-rRNA 45S (35S in yeast), which matures into 28S, 18S and 5.8S rRNAs, which will form the major RNA sections of the ribosome. RNA polymerase II synthesizes precursors of mRNAs and most snRNA and microRNAs. RNA polymerase III synthesizes tRNAs, rRNA 5S and other small RNAs found in the nucleus and cytosol. RNA polymerase IV synthesizes siRNA in plants. RNA polymerase V synthesizes RNAs involved in siRNA-directed heterochromatin formation in plants.

Contemplated herein, in some embodiments, are RNA pol II and RNA pol III promoters. Promoters that direct accurate initiation of transcription by an RNA polymerase II are referred to as RNA pol II promoters. Examples of RNA pol II promoters for use in accordance with the present disclosure include, without limitation, human cytomegalovirus promoters, human ubiquitin promoters, human histone H2A1 promoters and human inflammatory chemokine CXCL 1 promoters. Other RNA pol II promoters are also contemplated herein. Promoters that direct accurate initiation of transcription by an RNA polymerase III are referred to as RNA pol III promoters. Examples of RNA pol III promoters for use in accordance with the present disclosure include, without limitation, a U6 promoter, a H1 promoter and promoters of transfer RNAs, 5S ribosomal RNA (rRNA), and the signal recognition particle 7SL RNA.

In some embodiments, a promoter may be an inducible promoter. An inducible promoter is one that is characterized by initiating or enhancing transcriptional activity when in the presence of, influenced by or contacted by an inducer or inducing agent. An inducer, or inducing agent, may be endogenous or a normally exogenous condition, compound or protein that contacts an engineered nucleic acid in such a way as to be active in inducing transcriptional activity from the inducible promoter.

Engineered nucleic acids of the present disclosure may be produced using standard molecular biology methods (see, e.g., Green and Sambrook, Molecular Cloning, A Laboratory Manual, 2012, Cold Spring Harbor Press).

In some embodiments, engineered constructs and/or engineered nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

In some embodiments, engineered constructs and/or engineered nucleic acids are included within a vector. A vector is a nucleic acid (e.g., DNA) used as a vehicle to artificially carry genetic material (e.g., an engineered nucleic acid) into another cell where, for example, it can be replicated and/or expressed. In some embodiments, a vector is an episomal vector (see, e.g., Van Craenenbroeck K. et al. Eur. J. Biochem. 267, 5665, 2000, incorporated by reference herein). A non-limiting example of a vector is a plasmid. Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector.

Engineered constructs of the present disclosure may be expressed in a variety of cell types. In some embodiments, engineered constructs are expressed in mammalian cells. For example, in some embodiments, engineered constructs are expressed in human cells, primate cells (e.g., vero cells), rat cells (e.g., GH3 cells, OC23 cells) or mouse cells (e.g., MC3T3 cells). There are a variety of human cell lines, including, without limitation, HEK cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, engineered constructs are expressed in human embryonic kidney (HEK) cells (e.g., HEK 293 or HEK 293T cells). In some embodiments, engineered constructs are expressed in bacterial cells, yeast cells, insect cells or other types of cells. In some embodiments, engineered constructs are expressed in stem cells (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Cells of the present disclosure, in some embodiments, are modified. A modified cell is a cell that contains an exogenous nucleic acid or a nucleic acid that does not occur in nature. In some embodiments, a modified cell contains a mutation in a genomic nucleic acid. In some embodiments, a modified cell contains an exogenous independently replicating nucleic acid (e.g., an engineered nucleic acid present on an episomal vector). In some embodiments, a modified cell is produced by introducing a foreign or exogenous nucleic acid into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), or microinjection of purified DNA directly into the nucleus of the cell (see, e.g., Capecchi M. R. Cell. 1980 November; 22(2 Pt 2): 479-88).

In some embodiments, a cell is modified to overexpress an endogenous protein of interest (e.g., via introducing or modifying a promoter or other regulatory element near the endogenous gene that encodes the protein of interest to increase its expression level). In some embodiments, a cell is modified by mutagenesis. In some embodiments, a cell is modified by introducing a recombinant nucleic acid into the cell in order to produce a genetic change of interest (e.g., via insertion or homologous recombination).

In some embodiments, an engineered nucleic acid may be codon-optimized, for example, for expression in human cells or other types of cells. Codon optimization is a technique to maximize the protein expression in living organism by increasing the translational efficiency of gene of interest by transforming a DNA sequence of nucleotides of one species into a DNA sequence of nucleotides of another species. Methods of codon optimization are well-known.

Engineered constructs of the present disclosure may be transiently expressed or stably expressed. “Transient cell expression” refers to expression by a cell of a nucleic acid that is not integrated into the nuclear genome of the cell. By comparison, “stable cell expression” refers to expression by a cell of a nucleic acid that remains in the nuclear genome of the cell and its daughter cells. Typically, to achieve stable cell expression, a cell is co-transfected with a marker gene and an exogenous nucleic acid (e.g., engineered nucleic acid) that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Examples of marker genes and selection agents for use in accordance with the present disclosure include, without limitation, dihydrofolate reductase with methotrexate, glutamine synthetase with methionine sulphoximine, hygromycin phosphotransferase with hygromycin, puromycin N-acetyltransferase with puromycin, and neomycin phosphotransferase with Geneticin, also known as G418. Other marker genes/selection agents are contemplated herein.

Expression of nucleic acids in transiently-transfected and/or stably-transfected cells may be constitutive or inducible. Inducible promoters for use as provided herein are described above.

Mammalian cells (e.g., human cells) modified to comprise an engineered constructs of the present disclosure may be cultured (e.g., maintained in cell culture) using conventional mammalian cell culture methods (see, e.g., Phelan M. C. Curr Protoc Cell Biol. 2007 September; Chapter 1: Unit 1.1, incorporated by reference herein). For example, cells may be grown and maintained at an appropriate temperature and gas mixture (e.g., 37° C., 5% CO₂ for mammalian cells) in a cell incubator. Culture conditions may vary for each cell type. For example, cell growth media may vary in pH, glucose concentration, growth factors, and the presence of other nutrients. Growth factors used to supplement media are often derived from the serum of animal blood, such as fetal bovine serum (FBS), bovine calf serum, equine serum and/or porcine serum. In some embodiments, culture media used as provided herein may be commercially available and/or well-described (see, e.g., Birch J. R., R. G. Spier (Ed.) Encyclopedia of Cell Technology, Wiley. 411-424, 2000; Keen M. J. Cytotechnology 17: 125-132, 1995; Zang, et al. Bio/Technology. 13: 389-392, 1995). In some embodiments, chemically defined media is used.

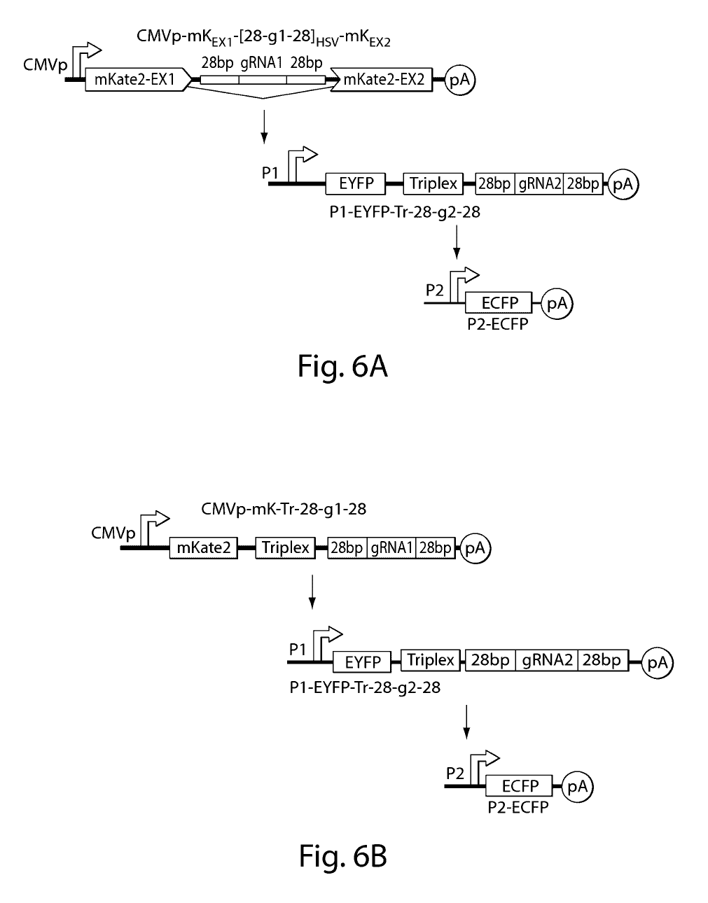

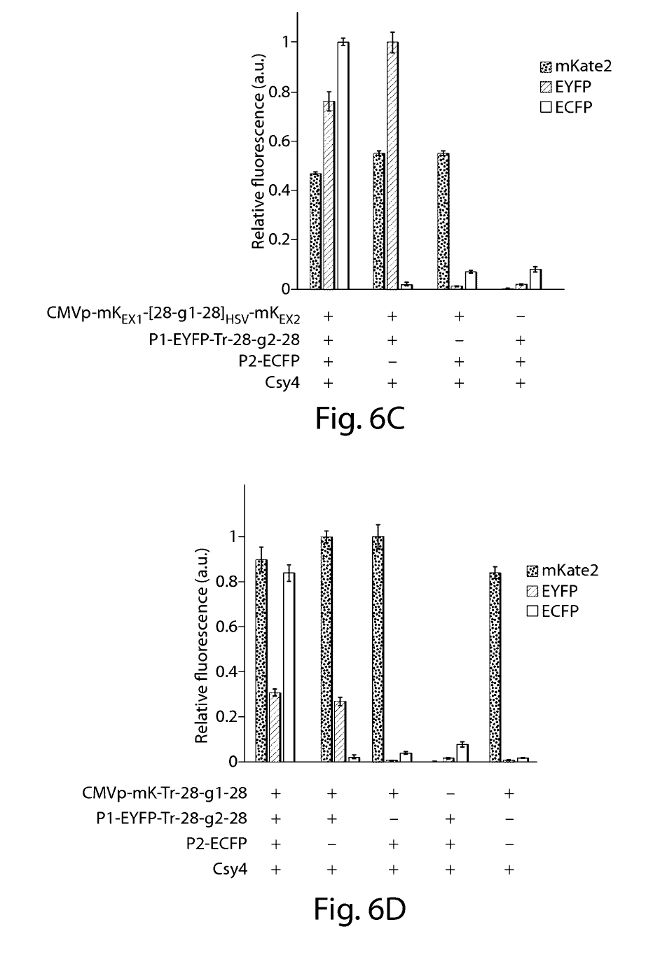

Also contemplated herein, in various aspects, are methods and compositions for constructing genetic circuits, including transcriptional cascades, within in a cell (e.g., a mammalian cell such as a human cell). Many complex gene circuits require the ability to implement cascades, in which signals integrated at one stage are transmitted into multiple downstream stages for processing and actuation. For example, gene cascades are important for synthetic-biology applications such as multi-layer artificial gene circuits that compute in living cells (Weber and Fussenegger, 2009). Transcriptional cascades are important in natural regulatory systems, such as those that control segmentation, sexual commitment and development (Dequeant and Pourquie, 2008; Peel et al., 2005; Sinha et al., 2014). FIGS. 6A and 6B provide non-limiting examples of how multiple engineered constructs of the present disclosure can be used together in a single cell to construct a transcriptional cascade.

As shown in FIG. 6A, a cell can be co-transfected, for example, with a first engineered construct having an ‘intron-Csy4’ configuration to express a first gRNA (‘gRNA1’) and mKate2, a second engineered construct having a ‘triplex-Csy4’ configuration to express a second gRNA (‘gRNA2’) and EYFP, and a third engineered construct configured to expresses ECFP. The cell also expresses Csy4 and a transcriptionally active Cas9 (taCas9). The engineered constructs are configured such that, when expressed in the presence of Csy4 ribonuclease, gRNA1 is released from the construct and guides a taCas9 protein to a complementary gRNA1 binding site within the promoter of the second engineered construct (and mKate2 is expressed). The taCas9 protein then activates transcription of the second engineered construct, thereby producing a second gRNA (‘gRNA2’) (and EYFP is expressed). gRNA then guides a taCas9 protein to a complementary gRNA2 binding site within the promoter of the third engineered construct. The taCas9 protein then activates transcription of the third engineered construct, which expresses ECFP.

As shown in FIG. 6B, a cell can be co-transfected, for example, with a two engineered constructs, each having a ‘triplex-Csy4’ configuration, wherein the gRNA (‘gRNA1’) encoded by the first construct is different from the gRNA (‘gRNA2’) encoded by the second construct. The mechanism of activation of each construct in FIG. 6B is similar to the mechanism described in FIG. 6A.

The present disclosure contemplates, in some embodiments, expression of multiple engineered constructs provided herein. For example, a cell may express 2 to 500, or more, different engineered constructs. In some embodiments, a cell may express 2 to 10, 2 to 25, 2 to 50, 2 to 75, 2 to 100, 2 to 200, 2 to 300 or 2 to 400 different engineered constructs. In some embodiments, a cell may express 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more different engineered constructs of the present disclosure. Engineered constructs are considered to be different from each other if the configuration of their genetic elements is different, as shown in FIG. 6A. Engineered constructs also are considered to be different from each other if the configuration of their genetic elements is the same but the particular elements differ, as shown in FIG. 6B.

It should be appreciated that the genetic elements provided herein, in some embodiments, are modular such that a cell may comprise multiple engineered constructs of the present disclosure, each construct comprising a different combination of elements configured in a different way, provided the elements are configured in a manner that permits transcriptional activation and subsequent nucleic acid expression. For example, an engineered construct may comprise a promoter (e.g., an RNA pol II promoter) operably linked to a nucleic acid that comprises: (a) a nucleotide sequence encoding at least one guide RNA (gRNA); and (b) one or more nucleotide sequences selected from (i) a nucleotide sequence encoding a protein of interest and (ii) a nucleotide sequence encoding an RNA interference molecule. Such engineered constructs may or may not further comprise cognate intronic splice sites flanking a gRNA or an RNA interference molecule (e.g., miRNA).

A nucleotide sequence encoding a gRNA may be flanked by ribonuclease recognition sites (e.g., Csy4 recognition sites) or a gRNA may be flanked by ribozymes. In some embodiments, an engineered construct includes a combination of nucleotide sequence encoding a gRNA flanked by ribonuclease recognition sites and a nucleotide sequence encoding a gRNA flanked by ribozymes. In some embodiments, an engineered construct includes a combination of a first nucleotide sequence encoding a gRNA flanked by ribonuclease recognition sites and a second nucleotide sequence encoding a gRNA flanked by ribozymes, wherein the first nucleotide sequence or the second nucleotide sequence is flanked by cognate intronic splice sites. In some embodiments, an engineered construct includes a combination of a first nucleotide sequence encoding a gRNA flanked by ribonuclease recognition sites and a second nucleotide sequence encoding a gRNA flanked by ribozymes, wherein the first nucleotide sequence and the second nucleotide sequence are each flanked by cognate intronic splice sites. In some embodiments, an engineered construct includes a combination of a first nucleotide sequence encoding a gRNA flanked by ribonuclease recognition sites and/or a second nucleotide sequence encoding a gRNA flanked by ribozymes, and an additional nucleotide sequence encoding a gRNA (flanked or not flanked by ribonuclease recognition sites or ribozymes) flanked by cognate intronic splice sites.

A nucleotide sequence encoding a protein of interest, in some embodiments, may also encode a gRNA flanked by ribonuclease recognition sites, which are flanked by cognate intronic splice sites. In some embodiments, a gRNA flanked by ribonuclease recognition sites may also encode an RNA interference molecule (e.g., miRNA and/or siRNA) within the protein of interest.

Engineered constructs of the present disclosure may or may not include a nucleotide sequence encoding a triple helix structure, depending on the particular configuration and stability of the constructs.

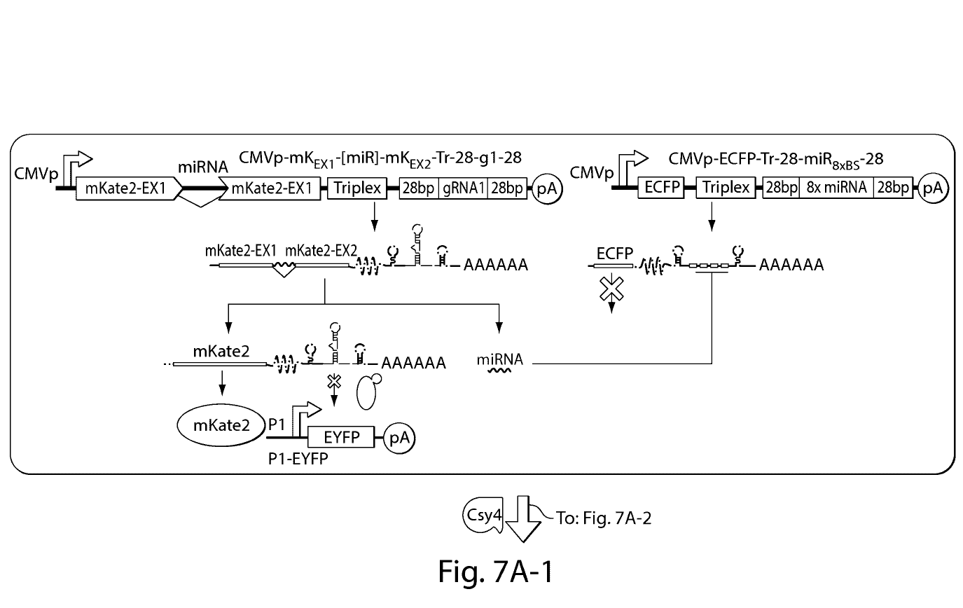

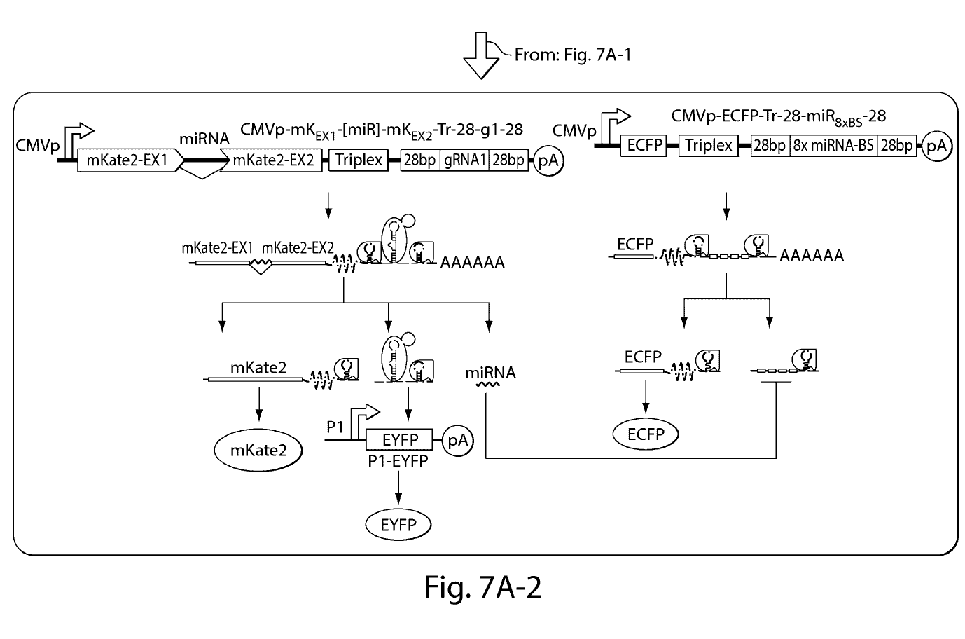

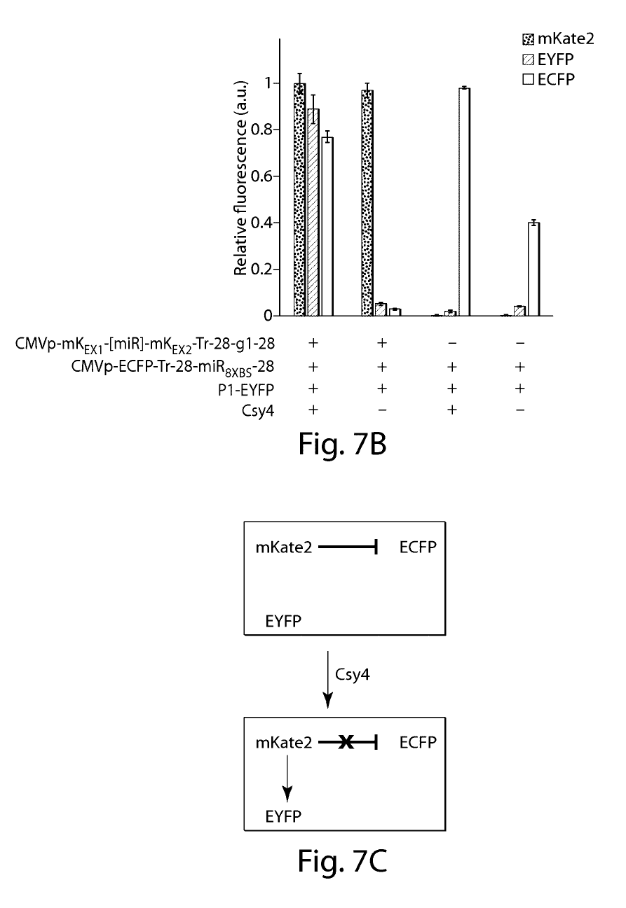

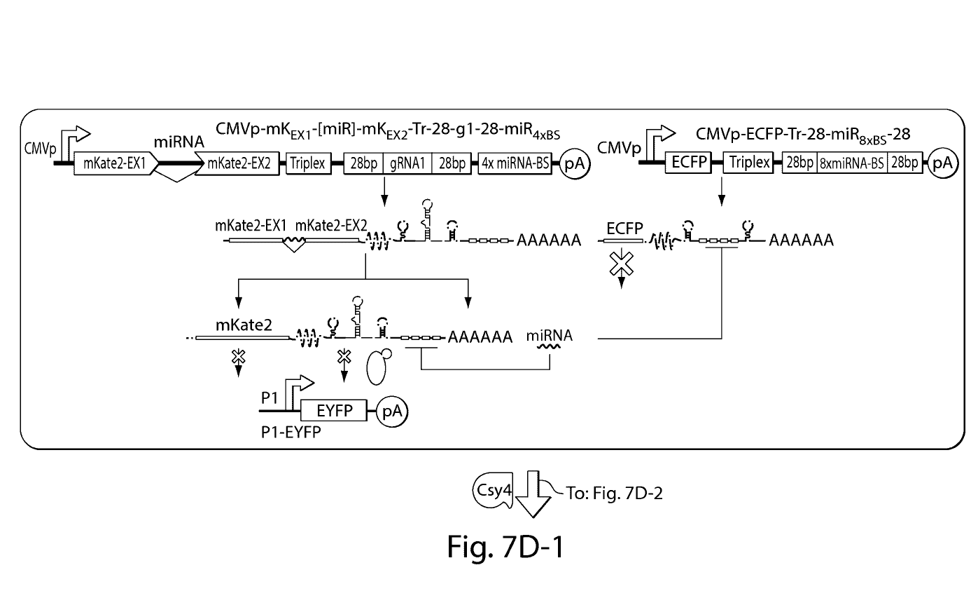

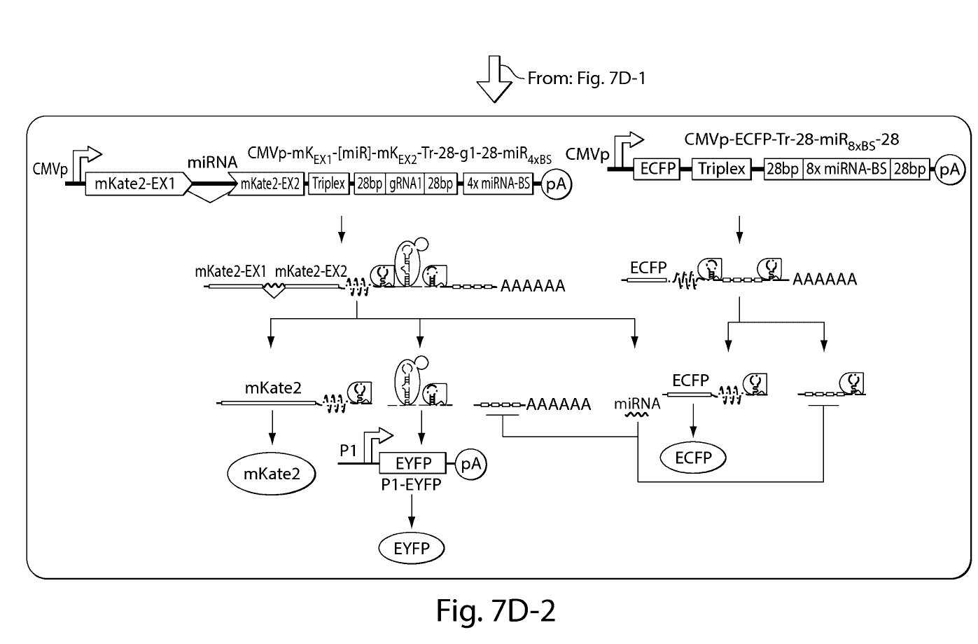

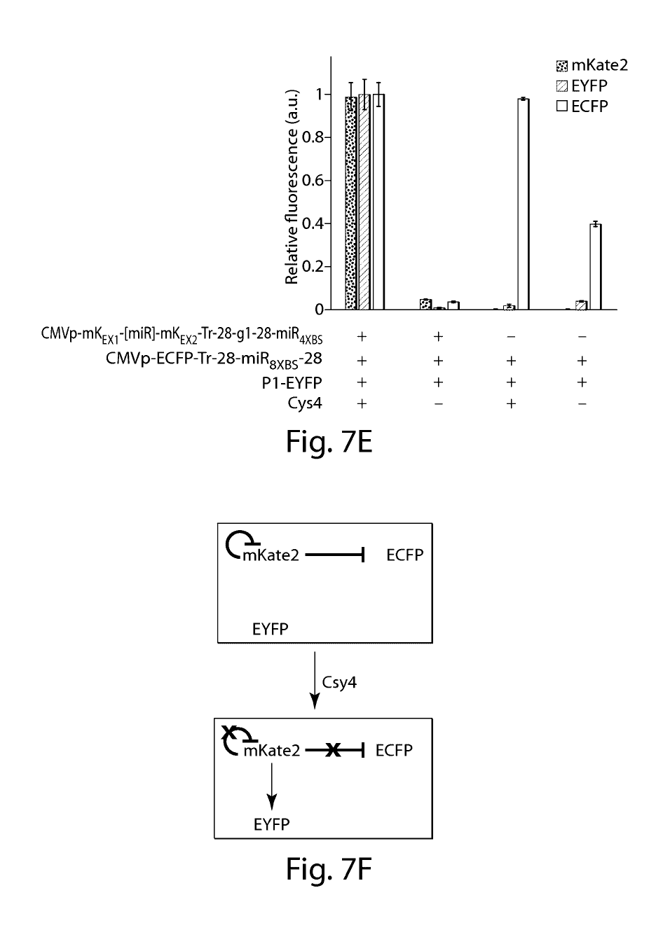

Also contemplated herein, in various aspects, are methods and compositions for “rewiring” cellular regulatory circuits. CRISPR transcription factor-based regulation can be integrated with RNA interference, for example, to inactivate repressive outputs and/or to activate otherwise inactive outputs. As shown in FIGS. 7A-7F, integrated methods of the present disclosure can be used to rewire multiple interconnections and feedback loops between genetic components, resulting in synchronized shifts in circuit behavior.

Thus, various aspects and embodiments of the present disclosure may be used to facilitate the construction of multi-mechanism genetic circuits that integrate RNA interference and CRISPR-based systems for tunable, multi-output gene regulation. Furthermore, ribonuclease-based RNA processing can be used to rewire multiple interconnections and feedback loops between genetic components, resulting in synchronized shifts in circuit behavior.

### Examples

### Example 1

### Functional gRNA Generation with an RNA Triple Helix and Csy4

An important first step to enabling complex CRISPR-TF-based circuits is to generate functional gRNAs from RNAP II promoters in human cells, which permits coupling of gRNA production to specific regulatory signals. For example, the activation of gRNA-dependent circuits can be initiated in defined cell types or states, or in response to external inputs. Furthermore, the ability to simultaneously express gRNAs along with proteins from a single transcript is beneficial. This enables multiple outputs, including effector proteins and regulatory links, to be produced from a concise genetic configuration. It can also enable the integration of gRNA expression into endogenous loci. Thus, the present Example demonstrates a system in which functional gRNAs and proteins are simultaneously produced by endogenous RNAP II promoters.

The RNA-binding and RNA-endonuclease capabilities of the Csy4 protein from P. aeruginosa (Haurwitz et al., 2012; Sternberg et al., 2012) were utilized in this example. Csy4 recognizes a 28 nucleotide RNA sequence (hereafter referred to as the ‘28’ sequence), cleaves the RNA, and remains bound to the upstream RNA fragment (Haurwitz et al., 2012). Thus, Csy4 was utilized to release gRNAs from transcripts generated by RNAP II promoters, which also encode functional protein sequences. To generate a gRNA-containing transcript, the potent CMV promoter (CMVp) was used to express the mKate2 protein. A gRNA (gRNA1), flanked by two Csy4 binding sites, was encoded downstream of the coding region of mKate2 (FIG. 1A). In this configuration, RNA cleavage by Csy4 releases a functional gRNA but also removes the poly-(A) tail from the upstream mRNA (encoding mKate2 in this case), resulting in impaired translation of most eukaryotic mRNAs (Jackson, 1993; Proudfoot, 2011).

To enable efficient translation of mRNA lacking a poly-(A) tail, a triple helix structure was used to functionally complement the loss of the poly-(A). A 110 bp fragment derived from the 3′ end of the mouse MALAT1 locus (Wilusz et al., 2012) was cloned downstream of mKate2 and upstream of the gRNA sequence flanked by Csy4 recognition sites. The MALAT1 lncRNA is deregulated in many human cancers (Lin et al., 2006) and despite lacking a poly-(A) tail, the MALAT1 is a stable transcript (Wilusz et al., 2008; Wilusz et al., 2012) that is protected from the exosome and 3′-5′ exonucleases by a highly conserved 3′ triple helical structure (triplex) (Wilusz et al., 2012). Thus, the final ‘triplex/Csy4’ configuration was a CMVp-driven mKate2 transcript with a 3′ triplex sequence followed by a 28-gRNA-28 sequence (CMVp-mK-Tr-28-gRNA-28) (FIG. 1A).

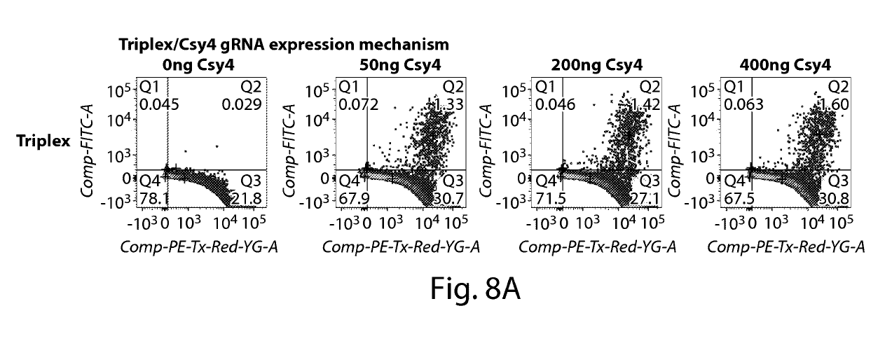

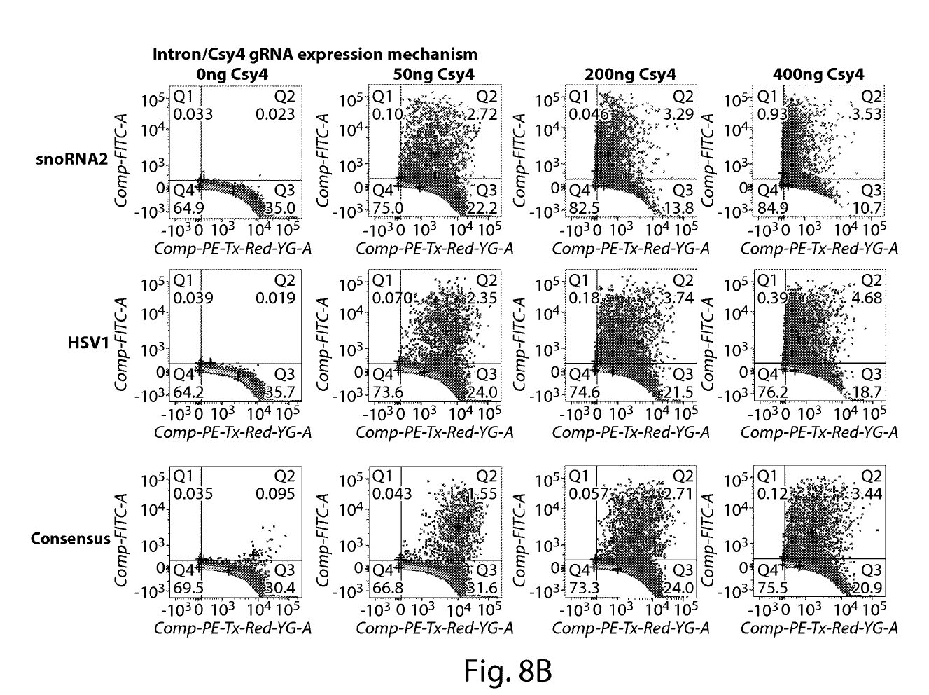

To characterize gRNA activity, HEK-293T cells were co-transfected with the CMVp-mK-Tr-28-gRNA1-28 expression plasmid, along with a plasmid encoding a synthetic P1 promoter that is specifically activated by gRNA1 to express EYFP. The P1 promoter contains 8× binding sites for gRNA1 and is based on a minimal promoter construct (Farzadfard et al., 2013). In this experiment and those that follow (unless otherwise indicated), the cells were co-transfected with a transcriptionally active dCas9-NLS-VP64 protein (taCas9) expressed by a CMV promoter. HEK-293T cells were co-transfected with 0-400 ng of a Csy4-expressing plasmid (where Csy4 was produced by the murine PGK1 promoter) along with 1 μg of the other plasmids (FIG. 1B and FIG. 8A for raw data).

Increasing Csy4 concentration levels did not result in a decrease of mKate2 levels, but instead led to an up to 5-fold increase (FIG. 1B). Furthermore, functional gRNAs generated from this construct induced EYFP expression by up to 60-fold from the P1 promoter. While mKate2 expression continued to increase with the concentration of the Csy4-expressing plasmid, EYFP activation plateaued after 50 ng of the Csy4-producing plasmid. In addition, there was evidence of cytotoxicity at 400 ng Csy4 plasmid concentrations. Thus, 100-200 ng of the Csy4 plasmid was used in subsequent experiments (except where otherwise noted), although this reduced the number of Csy4-positive cells after transfection. Alternatively, weaker promoters can be used to reduce Csy4 expression levels or stable cell lines can be generated with low or moderate levels of Csy4.

Interestingly, although a 5′ Csy4 recognition site alone should be sufficient to release gRNAs from the RNA transcript, this variant configuration did not generate functional gRNAs capable of activating a downstream target promoter above background levels (data not shown). Without being bound by theory, this could be the result of RNA destabilization, poly-(A)-mediated cytoplasmic transport, interference of the poly-(A) tail with taCas9 activity, or other mechanisms.

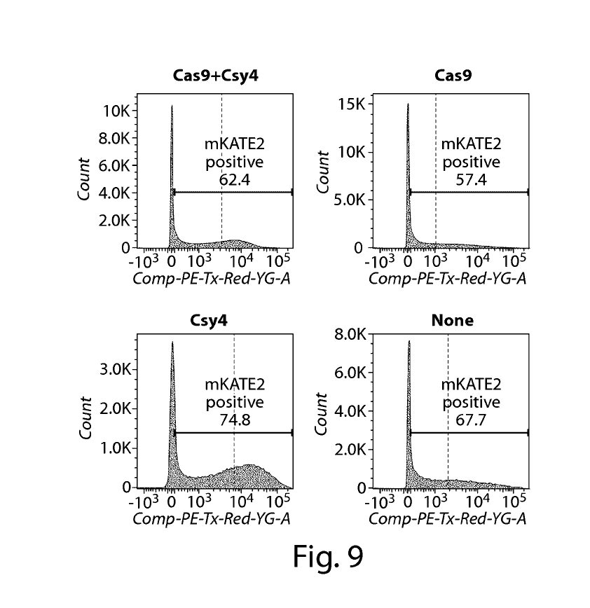

The relative effects of Csy4 and taCas9 on the expression of mKate2 were further characterized. mKate2 fluorescence was measured from the ‘triplex/Csy4’-based gRNA expression construct in the presence of Csy4 and taCas9, Csy4 alone, taCas9 alone, or neither protein (FIG. 1C and FIG. 9). The lowest mKate2 fluorescence levels resulted from the taCas9 only condition. Without being bound by theory, because a taCas9 with a strong nuclear localization sequence (NLS) was used, this effect could arise from taCas9 binding to the gRNA within the mRNA and localizing the transcript to the nucleus. This theory is supported by data demonstrating that endogenous promoters can be activated by gRNAs produced from the ‘triplex/Csy4’-based configuration even in the absence of Csy4 (see below and FIGS. 1D, 1E). The highest mKate2 expression levels were obtained with Csy4 alone, suggesting that Csy4 processing could enhance mKate2 levels. Expression of mKate2 in the absence of both Csy4 and taCas9 as well as in the presence of both Csy4 and taCas9 were similar and reduced by 3-4 fold compared with Csy4 only.

### Example 2

### Modulating Endogenous Loci with CRISPR-TFs Expressed from Human Promoters

To validate the robustness of the ‘triplex/Csy4’ configuration, it was adapted to regulate the expression of a native genomic target in human cells. The endogenous IL1RN locus was targeted for gene activation via the co-expression of four distinct gRNAs, gRNA3-6 (Table 1) (Perez-Pinera et al., 2013a).

TABLE 1
Sequences used in the study

| | Sequence (Kozak sequence and start |
|---|---|
| Name | codon underlined) |
| dCas9-3xNLS- | GCCACCATGGACAAGAAGTACTCCATTGGGCTCGCCATCGGCA |
| VP64-3′LTR | CAAACAGCGTCGGCTGGGCCGTCATTACGGACGAGTACAAGG |
| (Construct 1) | TGCCGAGCAAAAAATTCAAAGTTCTGGGCAATACCGATCGCC |
| | ACAGCATAAAGAAGAACCTCATTGGCGCCCTCCTGTTCGACTC |
| | CGGGGAGACGGCCGAAGCCACGCGGCTCAAAAGAACAGCACG |
| | GCGCAGATATACCCGCAGAAAGAATCGGATCTGCTACCTGCA |
| | GGAGATCTTTAGTAATGAGATGGCTAAGGTGGATGACTCTTTC |
| | TTCCATAGGCTGGAGGAGTCCTTTTTGGTGGAGGAGGATAAAA |
| | AGCACGAGCGCCACCCAATCTTTGGCAATATCGTGGACGAGGT |
| | GGCGTACCATGAAAAGTACCCAACCATATATCATCTGAGGAA |
| | GAAGCTTGTAGACAGTACTGATAAGGCTGACTTGCGGTTGATC |
| | TATCTCGCGCTGGCGCATATGATCAAATTTCGGGGACACTTCC |
| | TCATCGAGGGGGACCTGAACCCAGACAACAGCGATGTCGACA |
| | AACTCTTTATCCAACTGGTTCAGACTTACAATCAGCTTTTCGAA |
| | GAGAACCCGATCAACGCATCCGGAGTTGACGCCAAAGCAATC |
| | CTGAGCGCTAGGCTGTCCAAATCCCGGCGGCTCGAAAACCTCA |
| | TCGCACAGCTCCCTGGGGAGAAGAAGAACGGCCTGTTTGGTA |
| | ATCTTATCGCCCTGTCACTCGGGCTGACCCCCAACTTTAAATCT |
| | AACTTCGACCTGGCCGAAGATGCCAAGCTTCAACTGAGCAAA |
| | GACACCTACGATGATGATCTCGACAATCTGCTGGCCCAGATCG |
| | GCGACCAGTACGCAGACCTTTTTTTGGCGGCAAAGAACCTGTC |
| | AGACGCCATTCTGCTGAGTGATATTCTGCGAGTGAACACGGAG |
| | ATCACCAAAGCTCCGCTGAGCGCTAGTATGATCAAGCGCTATG |
| | ATGAGCACCACCAAGACTTGACTTTGCTGAAGGCCCTTGTCAG |
| | ACAGCAACTGCCTGAGAAGTACAAGGAAATTTTCTTCGATCAG |
| | TCTAAAAATGGCTACGCCGGATACATTGACGGCGGAGCAAGC |
| | CAGGAGGAATTTTACAAATTTATTAAGCCCATCTTGGAAAAAA |
| | TGGACGGCACCGAGGAGCTGCTGGTAAAGCTTAACAGAGAAG |
| | ATCTGTTGCGCAAACAGCGCACTTTCGACAATGGAAGCATCCC |
| | CCACCAGATTCACCTGGGCGAACTGCACGCTATCCTCAGGCGG |
| | CAAGAGGATTTCTACCCCTTTTTGAAAGATAACAGGGAAAAG |
| | ATTGAGAAAATCCTCACATTTCGGATACCCTACTATGTAGGCC |
| | CCCTCGCCCGGGGAAATTCCAGATTCGCGTGGATGACTCGCAA |
| | ATCAGAAGAGACCATCACTCCCTGGAACTTCGAGGAAGTCGT |
| | GGATAAGGGGGCCTCTGCCCAGTCCTTCATCGAAAGGATGACT |
| | AACTTTGATAAAAATCTGCCTAACGAAAAGGTGCTTCCTAAAC |
| | ACTCTCTGCTGTACGAGTACTTCACAGTTTATAACGAGCTCAC |
| | CAAGGTCAAATACGTCACAGAAGGGATGAGAAAGCCAGCATT |
| | CCTGTCTGGAGAGCAGAAGAAAGCTATCGTGGACCTCCTCTTC |
| | AAGACGAACCGGAAAGTTACCGTGAAACAGCTCAAAGAAGAC |
| | TATTTCAAAAAGATTGAATGTTTCGACTCTGTTGAAATCAGCG |
| | GAGTGGAGGATCGCTTCAACGCATCCCTGGGAACGTATCACG |
| | ATCTCCTGAAAATCATTAAAGACAAGGACTTCCTGGACAATGA |
| | GGAGAACGAGGACATTCTTGAGGACATTGTCCTCACCCTTACG |
| | TTGTTTGAAGATAGGGAGATGATTGAAGAACGCTTGAAAACTT |
| | ACGCTCATCTCTTCGACGACAAAGTCATGAAACAGCTCAAGAG |
| | GCGCCGATATACAGGATGGGGGCGGCTGTCAAGAAAACTGAT |
| | CAATGGGATCCGAGACAAGCAGAGTGGAAAGACAATCCTGGA |
| | TTTTCTTAAGTCCGATGGATTTGCCAACCGGAACTTCATGCAG |
| | TTGATCCATGATGACTCTCTCACCTTTAAGGAGGACATCCAGA |
| | AAGCACAAGTTTCTGGCCAGGGGGACAGTCTTCACGAGCACA |
| | TCGCTAATCTTGCAGGTAGCCCAGCTATCAAAAAGGGAATACT |
| | GCAGACCGTTAAGGTCGTGGATGAACTCGTCAAAGTAATGGG |
| | AAGGCATAAGCCCGAGAATATCGTTATCGAGATGGCCCGAGA |
| | GAACCAAACTACCCAGAAGGGACAGAAGAACAGTAGGGAAA |
| | GGATGAAGAGGATTGAAGAGGGTATAAAAGAACTGGGGTCCC |
| | AAATCCTTAAGGAACACCCAGTTGAAAACACCCAGCTTCAGA |
| | ATGAGAAGCTCTACCTGTACTACCTGCAGAACGGCAGGGACA |
| | TGTACGTGGATCAGGAACTGGACATCAATCGGCTCTCCGACTA |
| | CGACGTGGATGCCATCGTGCCCCAGTCTTTTCTCAAAGATGAT |
| | TCTATTGATAATAAAGTGTTGACAAGATCCGATAAAAATAGAG |
| | GGAAGAGTGATAACGTCCCCTCAGAAGAAGTTGTCAAGAAAA |
| | TGAAAAATTATTGGCGGCAGCTGCTGAACGCCAAACTGATCAC |
| | ACAACGGAAGTTCGATAATCTGACTAAGGCTGAACGAGGTGG |
| | CCTGTCTGAGTTGGATAAAGCCGGCTTCATCAAAAGGCAGCTT |
| | GTTGAGACACGCCAGATCACCAAGCACGTGGCCCAAATTCTCG |
| | ATTCACGCATGAACACCAAGTACGATGAAAATGACAAACTGA |
| | TTCGAGAGGTGAAAGTTATTACTCTGAAGTCTAAGCTGGTCTC |
| | AGATTTCAGAAAGGACTTTCAGTTTTATAAGGTGAGAGAGATC |
| | AACAATTACCACCATGCGCATGATGCCTACCTGAATGCAGTGG |
| | TAGGCACTGCACTTATCAAAAAATATCCCAAGCTTGAATCTGA |
| | ATTTGTTTACGGAGACTATAAAGTGTACGATGTTAGGAAAATG |
| | ATCGCAAAGTCTGAGCAGGAAATAGGCAAGGCCACCGCTAAG |
| | TACTTCTTTTACAGCAATATTATGAATTTTTTCAAGACCGAGAT |
| | TACACTGGCCAATGGAGAGATTCGGAAGCGACCACTTATCGA |
| | AACAAACGGAGAAACAGGAGAAATCGTGTGGGACAAGGGTA |
| | GGGATTTCGCGACAGTCCGGAAGGTCCTGTCCATGCCGCAGGT |
| | GAACATCGTTAAAAAGACCGAAGTACAGACCGGAGGCTTCTC |
| | CAAGGAAAGTATCCTCCCGAAAAGGAACAGCGACAAGCTGAT |
| | CGCACGCAAAAAAGATTGGGACCCCAAGAAATACGGCGGATT |
| | CGATTCTCCTACAGTCGCTTACAGTGTACTGGTTGTGGCCAAA |
| | GTGGAGAAAGGGAAGTCTAAAAAACTCAAAAGCGTCAAGGAA |
| | CTGCTGGGCATCACAATCATGGAGCGATCAAGCTTCGAAAAA |
| | AACCCCATCGACTTTCTCGAGGCGAAAGGATATAAAGAGGTC |
| | AAAAAAGACCTCATCATTAAGCTTCCCAAGTACTCTCTCTTTG |
| | AGCTTGAAAACGGCCGGAAACGAATGCTCGCTAGTGCGGGCG |
| | AGCTGCAGAAAGGTAACGAGCTGGCACTGCCCTCTAAATACG |
| | TTAATTTCTTGTATCTGGCCAGCCACTATGAAAAGCTCAAAGG |
| | GTCTCCCGAAGATAATGAGCAGAAGCAGCTGTTCGTGGAACA |
| | ACACAAACACTACCTTGATGAGATCATCGAGCAAATAAGCGA |
| | ATTCTCCAAAAGAGTGATCCTCGCCGACGCTAACCTCGATAAG |
| | GTGCTTTCTGCTTACAATAAGCACAGGGATAAGCCCATCAGGG |
| | AGCAGGCAGAAAACATTATCCACTTGTTTACTCTGACCAACTT |
| | GGGCGCGCCTGCAGCCTTCAAGTACTTCGACACCACCATAGAC |
| | AGAAAGCGGTACACCTCTACAAAGGAGGTCCTGGACGCCACA |
| | CTGATTCATCAGTCAATTACGGGGCTCTATGAAACAAGAATCG |
| | ACCTCTCTCAGCTCGGTGGAGACAGCAGGGCTGACGGGCCCTC |
| | ACTGGGTTCAGGGTCACCCAAGAAGAAACGCAAAGTCGAGGA |
| | TCCAAAGAAGAAAAGGAAGGTTGAAGACCCCAAGAAAAAGA |
| | GGAAGGTGGATGGGATCGGCTCAGGCAGCAACGGCGGTGGAG |
| | GTTCAGACGCTTTGGACGATTTCGATCTCGATATGCTCGGTTCT |
| | GACGCCCTGGATGATTTCGATCTGGATATGCTCGGCAGCGACG |
| | CTCTCGACGATTTCGACCTCGACATGCTCGGGTCAGATGCCTT |
| | GGATGATTTTGACCTGGATATGCTCTCATGATGA |
| | (SEQ ID NO: 2) |
| PGK1p-Csy4-pA | GCCACCATGAAATCTTCTCACCATCACCATCACCATGAAAACC |
| (Construct 2) | TGTACTTCCAATCCAATGCAGCTAGCGACCACTATCTGGACAT |
| | CAGACTGAGGCCCGATCCTGAGTTCCCTCCCGCCCAGCTGATG |
| | AGCGTGCTGTTTGGCAAGCTGCATCAGGCTCTGGTCGCCCAAG |
| | GCGGAGACAGAATCGGCGTGTCCTTCCCCGACCTGGACGAGTC |
| | CCGGAGTCGCCTGGGCGAGCGGCTGAGAATCCACGCCAGCGC |
| | AGACGATCTGCGCGCCCTGCTGGCCCGGCCTTGGCTGGAGGGC |
| | CTGCGGGATCATCTGCAGTTTGGCGAGCCCGCCGTGGTGCCAC |
| | ACCCAACACCCTACCGCCAGGTGAGCCGCGTGCAGGCCAAGT |
| | CAAATCCCGAGAGACTGCGGCGGAGGCTGATGAGGCGACATG |
| | ATCTGAGCGAGGAGGAGGCCAGAAAGAGAATCCCCGACACAG |
| | TGGCCAGAGCCCTGGATCTGCCATTTGTGACCCTGCGGAGCCA |
| | GAGCACTGGCCAGCATTTCAGACTGTTCATCAGACACGGGCCC |
| | CTGCAGGTGACAGCCGAGGAGGGCGGATTTACATGCTATGGC |
| | CTGTCTAAAGGCGGCTTCGTGCCCTGGTTCTGA |
| | (SEQ ID NO: 3) |
| mKate2-Triplex-28- | GCCACCATGGTGTCTAAGGGCGAAGAGCTGATTAAGGAGAAC |
| gRNA1-28-pA | ATGCACATGAAGCTGTACATGGAGGGCACCGTGAACAACCAC |
| (Construct 3) | CACTTCAAGTGCACATCCGAGGGCGAAGGCAAGCCCTACGAG |
| | GGCACCCAGACCATGAGAATCAAGGTGGTCGAGGGCGGCCCT |
| | CTCCCCTTCGCCTTCGACATCCTGGCTACCAGCTTCATGTACGG |
| | CAGCAAAACCTTCATCAACCACACCCAGGGCATCCCCGACTTC |
| | TTTAAGCAGTCCTTCCCTGAGGGCTTCACATGGGAGAGAGTCA |
| | CCACATACGAAGACGGGGGCGTGCTGACCGCTACCCAGGACA |
| | CCAGCCTCCAGGACGGCTGCCTCATCTACAACGTCAAGATCAG |
| | AGGGGTGAACTTCCCATCCAACGGCCCTGTGATGCAGAAGAA |
| | AACACTCGGCTGGGAGGCCTCCACCGAGATGCTGTACCCCGCT |
| | GACGGCGGCCTGGAAGGCAGAAGCGACATGGCCCTGAAGCTC |
| | GTGGGCGGGGGCCACCTGATCTGCAACTTGAAGACCACATAC |
| | AGATCCAAGAAACCCGCTAAGAACCTCAAGATGCCCGGCGTC |
| | TACTATGTGGACAGAAGACTGGAAAGAATCAAGGAGGCCGAC |
| | AAAGAGACCTACGTCGAGCAGCACGAGGTGGCTGTGGCCAGA |
| | TACTGCGACCTCCCTAGCAAACTGGGGCACAAACTTAATTGAT |
| | AAACCGGTGATTCGTCAGTAGGGTTGTAAAGGTTTTTCTTTTCC |
| | TGAGAAAACAACCTTTTGTTTTCTCAGGTTTTGCTTTTTGGCCT |
| | TTCCCTAGCTTTAAAAAAAAAAAAGCAAAACTCACCGAGGCA |
| | GTTCCATAGGATGGCAAGATCCTGGTATTGGTCTGCGAGTTCA |
| | CTGCCGTATAGGCAGCTAAGAAATAGTCGCGTGTAGCGAAGC |
| | AGTTTTAGAGCTAGAAATAGCAAGTTAAAATAAGGCTAGTCC |
| | GTTATCAACTTGAAAAAGTGGCACCGAGTCGGTGCTTTTTTTC |
| | GTTCACTGCCGTATAGGCAGCTAAGAAACAAACAGGAATCGA |
| | ATGCAACCGGCGCAGGAACACTGCCAGCGCATCAACCCCGGG |
| | (SEQ ID NO: 4) |
| mKate2_EX1-[28- | GCCACCATGGTGTCTAAGGGCGAAGAGCTGATTAAGGAGAAC |
| gRNA1-28]_{Hsv1}- | ATGCACATGAAGCTGTACATGGAGGGCACCGTGAACAACCAC |
| mKate2_EX2-pA | CACTTCAAGTGCACATCCGAGGGCGAAGGCAAGCCCTACGAG |
| (Construct 4) | GGCACCCAGACCATGAGAATCAAGGTGGTCGAGGGCGGCCCT |
| | CTCCCCTTCGCCTTCGACATCCTGGCTACCAGCTTCATGTACGG |
| | CAGCAAAACCTTCATCAACCACACCCAGGGCATCCCCGACTTC |
| | TTTAAGCAGTCCTTCCCTGAGGTAAGTGTTCACTGCCGTATAG |
| | GCAGCTAAGAAATAGTCGCGTGTAGCGAAGCAGTTTTAGAGC |
| | TAGAAATAGCAAGTTAAAATAAGGCTAGTCCGTTATCAACTTG |
| | AAAAAGTGGCACCGAGTCGGTGCTTTTTTTCGTTCACTGCCGT |
| | ATAGGCAGCTAAGAAAGAGGGAGTCGAGTCTTCTTTTTTTTTT |
| | TCACAGGGCTTCACATGGGAGAGAGTCACCACATACGAAGAC |
| | GGGGGCGTGCTGACCGCTACCCAGGACACCAGCCTCCAGGAC |
| | GGCTGCCTCATCTACAACGTCAAGATCAGAGGGGTGAACTTCC |
| | CATCCAACGGCCCTGTGATGCAGAAGAAAACACTCGGCTGGG |
| | AGGCCTCCACCGAGATGCTGTACCCCGCTGACGGCGGCCTGGA |
| | AGGCAGAAGCGACATGGCCCTGAAGCTCGTGGGCGGGGGCCA |
| | CCTGATCTGCAACTTGAAGACCACATACAGATCCAAGAAACCC |
| | GCTAAGAACCTCAAGATGCCCGGCGTCTACTATGTGGACAGA |
| | AGACTGGAAAGAATCAAGGAGGCCGACAAAGAGACCTACGTC |
| | GAGCAGCACGAGGTGGCTGTGGCCAGATACTGCGACCTCCCT |
| | AGCAAACTGGGGCACAAACTTAATTGA |
| | (SEQ ID NO: 5) |
| P1-EYFP-pA | GCTAGCCATGCTTCGCTACACGCGACTATTAATATTTTCAGGC |
| (Construct 5) | TAGCCATGCTTCGCTACACGCGACTATTAATATTTTCAGGCTA |
| | GCCATGCTTCGCTACACGCGACTATTAATATTTTCAGGCTAGC |
| | CATGCTTCGCTACACGCGACTATTAATATTTTCAGGCTAGCCA |
| | TGCTTCGCTACACGCGACTATTAATATTTTCAGGCTAGCCATG |
| | CTTCGCTACACGCGACTATTAATATTTTCAGGCTAGCCATGCTT |
| | CGCTACACGCGACTATTAATATTTTCAGGCTAGCCATGCTTCG |
| | CTACACGCGACTATTAATATTTTCAGGCTAGCCATGCTTCGCT |
| | ACACGCGACTATTAATATTTTCAGGCTAGCGGGGGGCTATAAA |
| | AGGGGGTGGGGGCGTTCGTCCTGCTATCTAGCGTCGCGTTGAC |
| | CATGGCGCCACCATGAGCAGCGGCGCCCTGCTGTTCCACGGCA |
| | AGATCCCCTACGTGGTGGAGATGGAGGGCGATGTGGATGGCC |
| | ACACCTTCAGCATCCGCGGTAAGGGCTACGGCGATGCCAGCGT |
| | GGGCAAGGTGGATGCCCAGTTCATCTGCACCACCGGCGATGTG |
| | CCCGTGCCCTGGAGCACCCTGGTGACCACCCTGACCTACGGCG |
| | CCCAGTGCTTCGCCAAGTACGGCCCCGAGCTGAAGGATTTCTA |
| | CAAGAGCTGCATGCCCGATGGCTACGTGCAGGAGCGCACCAT |
| | CACCTTCGAGGGCGATGGCAATTTCAAGACCCGCGCCGAGGT |
| | GACCTTCGAGAATGGCAGCGTGTACAATCGCGTGAAGCTGAA |
| | TGGCCAGGGCTTCAAGAAGGATGGCCACGTGCTGGGCAAGAA |
| | TCTGGAGTTCAATTTCACCCCCCACTGCCTGTACATCTGGGGC |
| | GATCAGGCCAATCACGGCCTGAAGAGCGCCTTCAAGATCTGCC |
| | ACGAGATCGCCGGCAGCAAGGGCGATTTCATCGTGGCCGATC |
| | ACACCCAGATGAATACCCCCATCGGCGGCGGCCCCGTGCACGT |
| | GCCCGAGTACCACCACATGAGCTACCACGTGAAGCTGAGCAA |
| | GGATGTGACCGATCACCGCGATAATATGAGCCTGACGGAGAC |
| | CGTGCGCGCCGTGGATTGCCGCAAGACCTACCTGTAA |
| | (SEQ ID NO: 6) |
| P2-ECFP-pA | GCTAGCCCAGGACAGTACTCCGACTTACTTAATATTTTCAGGC |
| (Construct 6) | TAGCCCAGGACAGTACTCCGACTTACTTAATATTTTCAGGCTA |
| | GCCCAGGACAGTACTCCGACTTACTTAATATTTTCAGGCTAGC |
| | CCAGGACAGTACTCCGACTTACTTAATATTTTCAGGCTAGCCC |
| | AGGACAGTACTCCGACTTACTTAATATTTTCAGGCTAGCCCAG |
| | GACAGTACTCCGACTTACTTAATATTTTCAGGCTAGCCCAGGA |
| | CAGTACTCCGACTTACTTAATATTTTCAGGCTAGCCCAGGACA |
| | GTACTCCGACTTACTTAATATTTTCAGGCTAGCGGGGGGCTAT |
| | AAAAGGGGGTGGGGGCGTTCGTCCTGCTATCTAGCGTCGCGTT |
| | GACCATGGTGAGCAAGGGCGAGGAGCTGTTCACCGGGGTGGT |
| | GCCCATCCTGGTCGAGCTGGACGGCGACGTAAACGGCCACAA |
| | GTTCAGCGTGTCCGGCGAGGGCGAGGGCGATGCCACCTACGG |
| | CAAGCTGACCCTGAAGTTCATCTGCACCACCGGCAAGCTGCCC |
| | GTGCCCTGGCCCACCCTCGTGACCACCCTGACCTGGGGCGTGC |
| | AGTGCTTCGCCCGCTACCCCGACCACATGAAGCAGCACGACTT |
| | CTTCAAGTCCGCCATGCCCGAAGGCTACGTCCAGGAGCGCACC |
| | ATCTTCTTCAAGGACGACGGCAACTACAAGACCCGCGCCGAG |
| | GTGAAGTTCGAGGGCGACACCCTGGTGAACCGCATCGAGCTG |
| | AAGGGCATCGACTTCAAGGAGGACGGCAACATCCTGGGGCAC |
| | AAGCTGGAGTACAACGCCATCAGCGACAACGTCTATATCACC |
| | GCCGACAAGCAGAAGAACGGCATCAAGGCCAACTTCAAGATC |
| | CGCCACAACATCGAGGACGGCAGCGTGCAGCTCGCCGACCAC |
| | TACCAGCAGAACACCCCCATCGGCGACGGCCCCGTGCTGCTGC |
| | CCGACAACCACTACCTGAGCACCCAGTCCAAGCTGAGCAAAG |
| | ACCCCAACGAGAAGCGCGATCACATGGTCCTGCTGGAGTTCGT |
| | GACCGCCGCCGGGATCACTCTCGGCATGGACGAGCTGTACAA |
| | GTAA |
| | (SEQ ID NO: 7) |
| mKate2_EX1-[28- | GCCACCATGGTGTCTAAGGGCGAAGAGCTGATTAAGGAGAAC |
| gRNA1-28]_{consensus}- | ATGCACATGAAGCTGTACATGGAGGGCACCGTGAACAACCAC |
| mKate2_EX2-pA | CACTTCAAGTGCACATCCGAGGGCGAAGGCAAGCCCTACGAG |
| (Construct 8) | GGCACCCAGACCATGAGAATCAAGGTGGTCGAGGGCGGCCCT |
| | CTCCCCTTCGCCTTCGACATCCTGGCTACCAGCTTCATGTACGG |
| | CAGCAAAACCTTCATCAACCACACCCAGGGCATCCCCGACTTC |
| | TTTAAGCAGTCCTTCCCTGAGGTAAGTGTTCACTGCCGTATAG |
| | GCAGCTAAGAAATAGTCGCGTGTAGCGAAGCAGTTTTAGAGC |
| | TAGAAATAGCAAGTTAAAATAAGGCTAGTCCGTTATCAACTTG |
| | AAAAAGTGGCACCGAGTCGGTGCTTTTTTTCGTTCACTGCCGT |
| | ATAGGCAGCTAAGAAATACTAACTTCGAGTCTTCTTTTTTTTTT |
| | TCACAGGGCTTCACATGGGAGAGAGTCACCACATACGAAGAC |
| | GGGGGCGTGCTGACCGCTACCCAGGACACCAGCCTCCAGGAC |
| | GGCTGCCTCATCTACAACGTCAAGATCAGAGGGGTGAACTTCC |
| | CATCCAACGGCCCTGTGATGCAGAAGAAAACACTCGGCTGGG |
| | AGGCCTCCACCGAGATGCTGTACCCCGCTGACGGCGGCCTGGA |
| | AGGCAGAAGCGACATGGCCCTGAAGCTCGTGGGCGGGGGCCA |
| | CCTGATCTGCAACTTGAAGACCACATACAGATCCAAGAAACCC |
| | GCTAAGAACCTCAAGATGCCCGGCGTCTACTATGTGGACAGA |
| | AGACTGGAAAGAATCAAGGAGGCCGACAAAGAGACCTACGTC |
| | GAGCAGCACGAGGTGGCTGTGGCCAGATACTGCGACCTCCCT |
| | AGCAAACTGGGGCACAAACTTAATTGACCCGGG |
| | (SEQ ID NO: 8) |
| mKate2_EX1-[28- | GCCACCATGGTGTCTAAGGGCGAAGAGCTGATTAAGGAGAAC |
| gRNA1-28]_{snoRNA2}- | ATGCACATGAAGCTGTACATGGAGGGCACCGTGAACAACCAC |
| mKate2_EX2-pA | CACTTCAAGTGCACATCCGAGGGCGAAGGCAAGCCCTACGAG |
| (Construct 9) | GGCACCCAGACCATGAGAATCAAGGTGGTCGAGGGCGGCCCT |
| | CTCCCCTTCGCCTTCGACATCCTGGCTACCAGCTTCATGTACGG |
| | CAGCAAAACCTTCATCAACCACACCCAGGGCATCCCCGACTTC |
| | TTTAAGCAGTCCTTCCCTGAGGTAAGTGTTCATTTCTCAAAAG |
| | ACCCTAATGTTCTTCCTTTACAGGAATGAATACTGTGCATGGA |
| | CCAATGATGACTTCCATACATGCATTCCTTGGAAAGCTGAACA |
| | AAATGAGTGGGAACTCTGTACTATCATCTTAGTTGAACTGAGG |
| | TCCGGATCCGTTCACTGCCGTATAGGCAGCTAAGAAATAGTCG |
| | CGTGTAGCGAAGCAGTTTTAGAGCTAGAAATAGCAAGTTAAA |
| | ATAAGGCTAGTCCGTTATCAACTTGAAAAAGTGGCACCGAGTC |
| | GGTGCTTTTTTTCAGATCTGTTCACTGCCGTATAGGCAGCTAAG |
| | AAATCTAGATGGATCGATGATGACTTCCATATATACATTCCTT |
| | GGAAAGCTGAACAAAATGAGTGAAAACTCTATACCGTCATTCT |
| | CGTCGAACTGAGGTCCAACCGGTGCACATTACTCCAACAGGG |
| | GCTAGACAGAGAGGGCCAACATTGATTCGTTGACATGGGTGG |
| | CTGCAGTACTAACTTCGAGTCTTCTTTTTTTTTTTCACAGGGCT |
| | TCACATGGGAGAGAGTCACCACATACGAAGACGGGGGCGTGC |
| | TGACCGCTACCCAGGACACCAGCCTCCAGGACGGCTGCCTCAT |
| | CTACAACGTCAAGATCAGAGGGGTGAACTTCCCATCCAACGG |
| | CCCTGTGATGCAGAAGAAAACACTCGGCTGGGAGGCCTCCAC |
| | CGAGATGCTGTACCCCGCTGACGGCGGCCTGGAAGGCAGAAG |
| | CGACATGGCCCTGAAGCTCGTGGGCGGGGGCCACCTGATCTGC |
| | AACTTGAAGACCACATACAGATCCAAGAAACCCGCTAAGAAC |
| | CTCAAGATGCCCGGCGTCTACTATGTGGACAGAAGACTGGAA |
| | AGAATCAAGGAGGCCGACAAAGAGACCTACGTCGAGCAGCAC |
| | GAGGTGGCTGTGGCCAGATACTGCGACCTCCCTAGCAAACTGG |
| | GGCACAAACTTAATTGA (SEQ ID NO: 9) |
| mKate2-Triplex- | GCCACCATGGTGTCTAAGGGCGAAGAGCTGATTAAGGAGAAC |
| HHRibo-gRNA1- | ATGCACATGAAGCTGTACATGGAGGGCACCGTGAACAACCAC |
| HDVRibo-pA | CACTTCAAGTGCACATCCGAGGGCGAAGGCAAGCCCTACGAG |
| (Construct 13) | GGCACCCAGACCATGAGAATCAAGGTGGTCGAGGGCGGCCCT |
| | CTCCCCTTCGCCTTCGACATCCTGGCTACCAGCTTCATGTACGG |
| | CAGCAAAACCTTCATCAACCACACCCAGGGCATCCCCGACTTC |
| | TTTAAGCAGTCCTTCCCTGAGGGCTTCACATGGGAGAGAGTCA |
| | CCACATACGAAGACGGGGGCGTGCTGACCGCTACCCAGGACA |
| | CCAGCCTCCAGGACGGCTGCCTCATCTACAACGTCAAGATCAG |
| | AGGGGTGAACTTCCCATCCAACGGCCCTGTGATGCAGAAGAA |
| | AACACTCGGCTGGGAGGCCTCCACCGAGATGCTGTACCCCGCT |
| | GACGGCGGCCTGGAAGGCAGAAGCGACATGGCCCTGAAGCTC |
| | GTGGGCGGGGGCCACCTGATCTGCAACTTGAAGACCACATAC |
| | AGATCCAAGAAACCCGCTAAGAACCTCAAGATGCCCGGCGTC |
| | TACTATGTGGACAGAAGACTGGAAAGAATCAAGGAGGCCGAC |
| | AAAGAGACCTACGTCGAGCAGCACGAGGTGGCTGTGGCCAGA |
| | TACTGCGACCTCCCTAGCAAACTGGGGCACAAACTTAATTGAT |
| | AAACCGGTGATTCGTCAGTAGGGTTGTAAAGGTTTTTCTTTTCC |
| | TGAGAAAACAACCTTTTGTTTTCTCAGGTTTTGCTTTTTGGCCT |
| | TTCCCTAGCTTTAAAAAAAAAAAAGCAAAACGACTACTGATG |
| | AGTCCGTGAGGACGAAACGAGTAAGCTCGTCTAGTCGCGTGT |
| | AGCGAAGCAGTTTTAGAGCTAGAAATAGCAAGTTAAAATAAG |
| | GCTAGTCCGTTATCAACTTGAAAAAGTGGCACCGAGTCGGTGC |
| | TTTTGGCCGGCATGGTCCCAGCCTCCTCGCTGGCGCCGGCTGG |
| | GCAACATGCTTCGGCATGGCGAATGGGACCCCGGG |
| | (SEQ ID NO: 10) |
| mKate2-HHRibo- | GCCACCATGGTGTCTAAGGGCGAAGAGCTGATTAAGGAGAAC |
| gRNA1-HDVRibo- | ATGCACATGAAGCTGTACATGGAGGGCACCGTGAACAACCAC |
| pA | CACTTCAAGTGCACATCCGAGGGCGAAGGCAAGCCCTACGAG |
| (Construct 14) | GGCACCCAGACCATGAGAATCAAGGTGGTCGAGGGCGGCCCT |
| | CTCCCCTTCGCCTTCGACATCCTGGCTACCAGCTTCATGTACGG |
| | CAGCAAAACCTTCATCAACCACACCCAGGGCATCCCCGACTTC |
| | TTTAAGCAGTCCTTCCCTGAGGGCTTCACATGGGAGAGAGTCA |
| | CCACATACGAAGACGGGGGCGTGCTGACCGCTACCCAGGACA |
| | CCAGCCTCCAGGACGGCTGCCTCATCTACAACGTCAAGATCAG |
| | AGGGGTGAACTTCCCATCCAACGGCCCTGTGATGCAGAAGAA |
| | AACACTCGGCTGGGAGGCCTCCACCGAGATGCTGTACCCCGCT |
| | GACGGCGGCCTGGAAGGCAGAAGCGACATGGCCCTGAAGCTC |
| | GTGGGCGGGGGCCACCTGATCTGCAACTTGAAGACCACATAC |
| | AGATCCAAGAAACCCGCTAAGAACCTCAAGATGCCCGGCGTC |
| | TACTATGTGGACAGAAGACTGGAAAGAATCAAGGAGGCCGAC |
| | AAAGAGACCTACGTCGAGCAGCACGAGGTGGCTGTGGCCAGA |
| | TACTGCGACCTCCCTAGCAAACTGGGGCACAAACTTAATTGAT |
| | AAACCGGTCGACTACTGATGAGTCCGTGAGGACGAAACGAGT |
| | AAGCTCGTCTAGTCGCGTGTAGCGAAGCAGTTTTAGAGCTAGA |
| | AATAGCAAGTTAAAATAAGGCTAGTCCGTTATCAACTTGAAAA |
| | AGTGGCACCGAGTCGGTGCTTTTGGCCGGCATGGTCCCAGCCT |
| | CCTCGCTGGCGCCGGCTGGGCAACATGCTTCGGCATGGCGAAT |
| | GGGACCCCGGG |
| | (SEQ ID NO: 11) |
| HHRibo-gRNA1- | CGACTACTGATGAGTCCGTGAGGACGAAACGAGTAAGCTCGT |
| HDVRibo-pA | CTAGTCGCGTGTAGCGAAGCAGTTTTAGAGCTAGAAATAGCA |
| (Construct 15) | AGTTAAAATAAGGCTAGTCCGTTATCAACTTGAAAAAGTGGCA |
| | CCGAGTCGGTGCTTTTGGCCGGCATGGTCCCAGCCTCCTCGCT |
| | GGCGCCGGCTGGGCAACATGCTTCGGCATGGCGAATGGGACC |
| | CCGGG (SEQ ID NO: 12) |
| mKate2-Triplex-28- | GCCACCATGGTGTCTAAGGGCGAAGAGCTGATTAAGGAGAAC |
| gRNA3-28-gRNA4- | ATGCACATGAAGCTGTACATGGAGGGCACCGTGAACAACCAC |
| 28-gRNA5-28- | CACTTCAAGTGCACATCCGAGGGCGAAGGCAAGCCCTACGAG |
| gRNA6-28-pA | GGCACCCAGACCATGAGAATCAAGGTGGTCGAGGGCGGCCCT |
| (Construct 19) | CTCCCCTTCGCCTTCGACATCCTGGCTACCAGCTTCATGTACGG |
| | CAGCAAAACCTTCATCAACCACACCCAGGGCATCCCCGACTTC |
| | TTTAAGCAGTCCTTCCCTGAGGGCTTCACATGGGAGAGAGTCA |
| | CCACATACGAAGACGGGGGCGTGCTGACCGCTACCCAGGACA |
| | CCAGCCTCCAGGACGGCTGCCTCATCTACAACGTCAAGATCAG |
| | AGGGGTGAACTTCCCATCCAACGGCCCTGTGATGCAGAAGAA |
| | AACACTCGGCTGGGAGGCCTCCACCGAGATGCTGTACCCCGCT |
| | GACGGCGGCCTGGAAGGCAGAAGCGACATGGCCCTGAAGCTC |
| | GTGGGCGGGGGCCACCTGATCTGCAACTTGAAGACCACATAC |
| | AGATCCAAGAAACCCGCTAAGAACCTCAAGATGCCCGGCGTC |
| | TACTATGTGGACAGAAGACTGGAAAGAATCAAGGAGGCCGAC |
| | AAAGAGACCTACGTCGAGCAGCACGAGGTGGCTGTGGCCAGA |
| | TACTGCGACCTCCCTAGCAAACTGGGGCACAAACTTAATTGAT |
| | AAACCGGTGATTCGTCAGTAGGGTTGTAAAGGTTTTTCTTTTCC |
| | TGAGAAAACAACCTTTTGTTTTCTCAGGTTTTGCTTTTTGGCCT |
| | TTCCCTAGCTTTAAAAAAAAAAAAGCAAAACTCACCGAGGCA |
| | GTTCCATAGGATGGCAAGATCCTGGTATCGGTCTGCGAGTTCA |
| | CTGCCGTATAGGCAGCTAAGAAAGCTAGCGTGTACTCTCTGAG |
| | GTGCTCGTTTTAGAGCTAGAAATAGCAAGTTAAAATAAGGCTA |
| | GTCCGTTATCAACTTGAAAAAGTGGCACCGAGTCGGTGCTTTT |
| | TTTCGTTCACTGCCGTATAGGCAGCTAAGAAAAGGTGACGCAG |
| | ATAAGAACCAGTTGTTTTAGAGCTAGAAATAGCAAGTTAAAAT |
| | AAGGCTAGTCCGTTATCAACTTGAAAAAGTGGCACCGAGTCG |
| | GTGCTTTTTTTCGTTCACTGCCGTATAGGCAGCTAAGAAACAG |
| | GGCATCAAGTCAGCCATCAGCGTTTTAGAGCTAGAAATAGCA |
| | AGTTAAAATAAGGCTAGTCCGTTATCAACTTGAAAAAGTGGCA |
| | CCGAGTCGGTGCTTTTTTTCGTTCACTGCCGTATAGGCAGCTAA |
| | GAAAAGTCGGGAGTCACCCTCCTGGAAACGTTTTAGAGCTAG |
| | AAATAGCAAGTTAAAATAAGGCTAGTCCGTTATCAACTTGAAA |
| | AAGTGGCACCGAGTCGGTGCTTTTTTTCGTTCACTGCCGTATA |
| | GGCAGCTAAGAAACCCGGG (SEQ ID NO: 13) |
| CMVp-mK_{Ex1}- | GCCACCATGGTGTCTAAGGGCGAAGAGCTGATTAAGGAGAAC |
| [miR]-mK_{Ex2}-Tr-28- | ATGCACATGAAGCTGTACATGGAGGGCACCGTGAACAACCAC |
| g1-28 | CACTTCAAGTGCACATCCGAGGGCGAAGGCAAGCCCTACGAG |
| (Construct 20) | GGCACCCAGACCATGAGAATCAAGGTGGTCGAGGGCGGCCCT |
| | CTCCCCTTCGCCTTCGACATCCTGGCTACCAGCTTCATGTACGG |
| | CAGCAAAACCTTCATCAACCACACCCAGGGCATCCCCGACTTC |
| | TTTAAGCAGTCCTTCCCTGAGGTAAGTGTGCTCGCTTCGGCAG |
| | CACATATACTATGTTGAATGAGGCTTCAGTACTTTACAGAATC |
| | GTTGCCTGCACATCTTGGAAACACTTGCTGGGATTACTTCTTCA |
| | GGTTAACCCAACAGAAGGCTCGAGTGCTGTTGACAGTGAGCG |
| | CCGCTTGAAGTCTTTAATTAAATAGTGAAGCCACAGATGTATT |
| | TAATTAAAGACTTCAAGCGGTGCCTACTGCCTCGGAGAATTCA |
| | AGGGGCTACTTTAGGAGCAATTATCTTGTTTACTAAAACTGAA |
| | TACCTTGCTATCTCTTTGATACATTTTTACAAAGCTGAATTAAA |
| | ATGGTATAAATTAAATCACTTTTTTCAATTGTACTAACTTCGAG |
| | TCTTCTTTTTTTTTTTCACAGGGCTTCACATGGGAGAGAGTCAC |
| | CACATACGAAGACGGGGGCGTGCTGACCGCTACCCAGGACAC |
| | CAGCCTCCAGGACGGCTGCCTCATCTACAACGTCAAGATCAGA |
| | GGGGTGAACTTCCCATCCAACGGCCCTGTGATGCAGAAGAAA |
| | ACACTCGGCTGGGAGGCCTCCACCGAGATGCTGTACCCCGCTG |
| | ACGGCGGCCTGGAAGGCAGAAGCGACATGGCCCTGAAGCTCG |
| | TGGGCGGGGGCCACCTGATCTGCAACTTGAAGACCACATACA |
| | GATCCAAGAAACCCGCTAAGAACCTCAAGATGCCCGGCGTCT |
| | ACTATGTGGACAGAAGACTGGAAAGAATCAAGGAGGCCGACA |
| | AAGAGACCTACGTCGAGCAGCACGAGGTGGCTGTGGCCAGAT |
| | ACTGCGACCTCCCTAGCAAACTGGGGCACAAACTTAATTGATA |
| | AACCGGTGATTCGTCAGTAGGGTTGTAAAGGTTTTTCTTTTCCT |
| | GAGAAAACAACCTTTTGTTTTCTCAGGTTTTGCTTTTTGGCCTT |
| | TCCCTAGCTTTAAAAAAAAAAAAGCAAAACTCACCGAGGCAG |
| | TTCCATAGGATGGCAAGATCCTGGTATCGGTCTGCGAGTTCAC |
| | TGCCGTATAGGCAGCTAAGAAATAGTCGCGTGTAGCGAAGCA |
| | GTTTTAGAGCTAGAAATAGCAAGTTAAAATAAGGCTAGTCCGT |
| | TATCAACTTGAAAAAGTGGCACCGAGTCGGTGCTTTTTTTCCC |
| | GCTTGAAGTCTTTAATTAAACCGCTTGAAGTCTTTAATTAAAC |
| | CGCTTGAAGTCTTTAATTAAACCGCTTGAAGTCTTTAATTAAA |
| | GTTCACTGCCGTATAGGCAGCTAAGAAACCCGGG |
| | (SEQ ID NO: 14) |
| ECFP-Triplex-28- | GCCACCATGGTGAGCAAGGGCGAGGAGCTGTTCACCGGGGTG |
| 8xmiRNA-BS-28- | GTGCCCATCCTGGTCGAGCTGGACGGCGACGTAAACGGCCAC |
| pA | AAGTTCAGCGTGTCCGGCGAGGGCGAGGGCGATGCCACCTAC |
| (Construct 22) | GGCAAGCTGACCCTGAAGTTCATCTGCACCACCGGCAAGCTGC |
| | CCGTGCCCTGGCCCACCCTCGTGACCACCCTGACCTGGGGCGT |
| | GCAGTGCTTCGCCCGCTACCCCGACCACATGAAGCAGCACGAC |
| | TTCTTCAAGTCCGCCATGCCCGAAGGCTACGTCCAGGAGCGCA |
| | CCATCTTCTTCAAGGACGACGGCAACTACAAGACCCGCGCCGA |
| | GGTGAAGTTCGAGGGCGACACCCTGGTGAACCGCATCGAGCT |
| | GAAGGGCATCGACTTCAAGGAGGACGGCAACATCCTGGGGCA |
| | CAAGCTGGAGTACAACGCCATCAGCGACAACGTCTATATCACC |
| | GCCGACAAGCAGAAGAACGGCATCAAGGCCAACTTCAAGATC |
| | CGCCACAACATCGAGGACGGCAGCGTGCAGCTCGCCGACCAC |
| | TACCAGCAGAACACCCCCATCGGCGACGGCCCCGTGCTGCTGC |
| | CCGACAACCACTACCTGAGCACCCAGTCCAAGCTGAGCAAAG |
| | ACCCCAACGAGAAGCGCGATCACATGGTCCTGCTGGAGTTCGT |
| | GACCGCCGCCGGGATCACTCTCGGCATGGACGAGCTGTACAA |
| | GTAAACCGGTGATTCGTCAGTAGGGTTGTAAAGGTTTTTCTTTT |
| | CCTGAGAAAACAACCTTTTGTTTTCTCAGGTTTTGCTTTTTGGC |
| | CTTTCCCTAGCTTTAAAAAAAAAAAAGCAAAACTCACCGAGG |
| | CAGTTCCATAGGATGGCAAGATCCTGGTATCGGTCTGCGAGTT |
| | CACTGCCGTATAGGCAGCTAAGAAACCGCTTGAAGTCTTTAAT |
| | TAAACCGCTTGAAGTCTTTAATTAAACCGCTTGAAGTCTTTAA |
| | TTAAACCGCTTGAAGTCTTTAATTAAACCTCTGGCCACATCGG |
| | TTCCTGCTCCGCTTGAAGTCTTTAATTAAACCGCTTGAAGTCTT |
| | TAATTAAACCGCTTGAAGTCTTTAATTAAACCGCTTGAAGTCT |
| | TTAATTAAAGTTCACTGCCGTATAGGCAGCTAAGAAACCCGGG |
| | (SEQ ID NO: 15) |
| Malat1 triple helix | GATTCGTCAGTAGGGTTGTAAAGGTTTTTCTTTTCCTGAGAAA |
| structure | ACAACCTTTTGTTTTCTCAGGTTTTGCTTTTTGGCCTTTCCCTAG |
| | CTTTAAAAAAAAAAAAGCAAAA (SEQ ID NO: 1) |
| Cys4 28 nt | GTTCACTGCCGTATAGGCAGCTAAGAAA |
| recognition site | (SEQ ID NO: 26) |
| gRNAs | where NNNNNNNNNNNNNNNNNNNN |
| | is one of the following: |
| gRNA1 | GAGTCGCGTGTAGCGAAGCA (SEQ ID NO: 16) |
| gRNA2 | GTAAGTCGGAGTACTGTCCT (SEQ ID NO: 17) |
| gRNA3 | GTGTACTCTCTGAGGTGCTC (SEQ ID NO: 18) |
| gRNA4 | GACGCAGATAAGAACCAGTT (SEQ ID NO: 19) |
| gRNA5 | GCATCAAGTCAGCCATCAGC (SEQ ID NO: 20) |
| gRNA6 | GGAGTCACCCTCCTGGAAAC (SEQ ID NO: 21) |

Each of the four gRNAs were designed to be expressed concomitantly with mKate2, each from a separate plasmid. Each set of four gRNAs was regulated by one of the following promoters (in descending order according to their activity level in HEK-293T cells): the Cytomegalovirus Immediate Early (CMVp), human Ubiquitin C (UbCp), human Histone H2A1 (H2A1p) (Rogakou et al., 1998), and human inflammatory chemokine CXCL1 (CXCL1p) promoters (Wang et al., 2006). As a control, the RNAP III promoter U6 (U6p) was used to drive expression of the four gRNAs. For each promoter tested, four plasmids encoding the four different gRNAs were co-transfected along with plasmids expressing taCas9 and Csy4. As a negative control, the IL1RN-targeting gRNA expression plasmids were substituted with plasmids that expressed gRNA1, which was non-specific for the IL1RN promoter (FIG. 1D, ‘NS’).

qRT-PCR was used to quantify the mRNA levels of the endogenous IL1RN gene, with the results normalized to the negative control. With the four gRNAs regulated by the U6 promoter, IL1RN activation levels were increased by 8,410-fold in the absence of Csy4 and 6,476-fold with 100 ng of the Csy4-expressing plasmid over the negative control (FIG. 1D, ‘U6p’). IL1RN activation with gRNAs expressed from the CMV promoter was substantial (FIG. 1D, ‘CMVp’), with 61-fold enhancement in the absence of Csy4 and 1539-fold enhancement with Csy4. The human RNAP II promoters generated ˜2-7 fold activation in the absence of Csy4 and ˜85-328-fold activation with Csy4 (FIG. 1D, ‘CXCL1p’, H2A1p′, ‘UbCp’).

To further characterize the input-output transfer function of endogenous gene regulation, mKate2 fluorescence generated by each promoter was used as a marker of input promoter activity for the various RNAP II promoters (FIG. 1E). The resulting transfer function was nearly linear in IL1RN activation over the range of mKate2 tested. This data indicates that IL1RN activation was not saturated in the conditions tested and that a large dynamic range of endogenous gene regulation can be achieved with human RNAP II promoters. Thus, tunable modulation of native genes can be achieved using CRISPR-TFs with gRNAs expressed from the ‘triplex/Csy4’ configuration.

### Example 3

### Functional gRNA Generation from Introns with Csy4

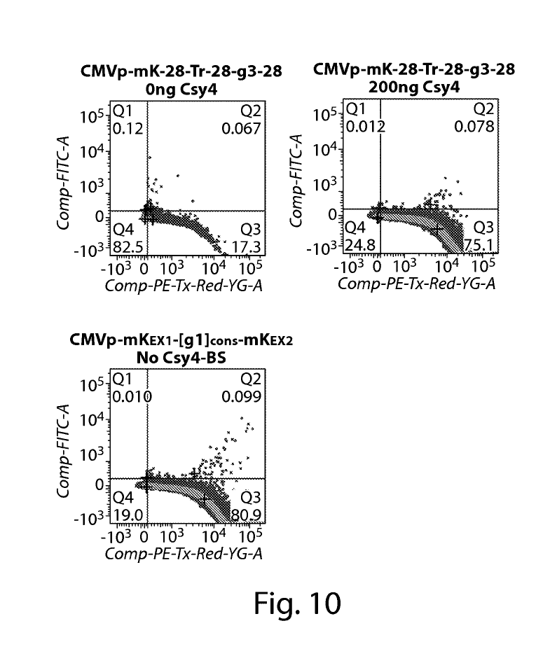

As a complement to the ‘triplex/Csy4’ configuration, an alternative strategy was developed for generating functional gRNAs from RNAP II promoters by encoding a gRNA within an intron in the coding sequence of a gene. Specifically, gRNA1 was encoded as an intron within the coding sequence of mKate2 (FIG. 2A) using ‘consensus’ acceptor, donor, and branching sequences (Smith et al., 1989; Taggart et al., 2012). Unexpectedly, this simple configuration resulted in undetectable EYFP levels (FIG. 10, bottom panel). Without being bound by theory, without any stabilization, intronic gRNAs appears to be rapidly degraded. To stabilize intronic gRNAs, intronic sequences that produce long-lived introns were used. These included sequences such as the HSV-1 latency associated intron, which forms a stable circular intron (Block and Hill, 1997), and the sno-lncRNA2 (snoRNA2) intron. The snoRNA2 intron is processed on both ends by the snoRNA machinery, which protects it from degradation and leads to the accumulation of IncRNAs flanked by snoRNA sequences which lack 5′ caps and 3′ poly-(A) tails. (Yin et al., 2012). However, these approaches for generating stable intronic gRNAs also resulted in undetectable activation of the target promoter (data not shown).

As an alternative strategy, intronic gRNAs were stabilized by flanking the gRNA cassette with two Csy4 recognition sites. Without being bound by theory, spliced gRNA-containing introns should be bound by Csy4, which should release functional gRNAs. In contrast to the ‘triplex/Csy4’ setting, Csy4 can also potentially bind and digest the pre-mRNA before splicing occurs. In this case, functional gRNA would be produced, but the mKate-containing pre-mRNA would be destroyed in the process (FIG. 2A). Thus, increased Csy4 concentrations would be expected to result in decreased mKate2 levels but greater levels of functional gRNA. Without being bound by theory, in this configuration, the decrease in mKate2 levels and increase in functional gRNA with Csy4 concentrations were expected to depend on several factors, which are illustrated in FIG. 2A (black lines, Csy4-independent processes; gray lines, Csy4-mediated processes). These competing factors include the rate at which Csy4 binds to its target sites and cleaves the RNA, the rate of splicing, and the rate of spliced gRNA degradation in the absence of Csy4. To examine the behavior of the ‘intron/Csy4’ configuration, the CMV promoter was used to drive expression of mKate2 with HSV1, snoRNA, and consensus introns containing gRNA1 flanked by two Csy4-binding-sites (CMVp-mKEX1-[28-g1-28]intron-mKEX2) along with a synthetic P1 promoter regulating the expression of EYFP (FIG. 2A).

The presence of Csy4 generated functional gRNA1, as determined by EYFP activation (FIGS. 2B-2D and FIG. 8B for raw data). gRNA1 generated from the HSV1 intron produced the strongest EYFP activation (FIG. 2D), which reached saturation at 200 ng of the Csy4 plasmid. In contrast, the snoRNA2 intron saturated EYFP expression at 50 ng of the Csy4 plasmid but the maximal EYFP levels produced by this intron were the lowest of all introns tested (˜65% of the HSV1 intron). In addition, increased Csy4 levels concomitantly reduced mKate2 levels. While these trends were similar for all three introns examined, the magnitudes of the effects were intron-specific. The snoRNA2 intron exhibited the largest decrease in mKate2 levels with increasing Csy4 plasmid concentrations, with a 15-fold reduction in mKate2 fluorescence at 400 ng of the Csy4 plasmid compared to the no Csy4 condition (FIG. 2C). The consensus and HSV1 introns exhibited mKate2 levels that were less sensitive to increasing Csy4 levels (FIGS. 2B and 2D). Thus, together with the ‘triplex/Csy4’ configuration, the ‘intron/Csy4’ approach provides a set of parts for the tunable production of functional gRNAs from translated genes. Specifically, absolute protein levels of the gRNA-containing genes and downstream target genes, as well as the ratios between them, can be determined by the choice of specific parts and concentration of Csy4.

### Example 4

### Interactions Between Csy4 and Intronic gRNA

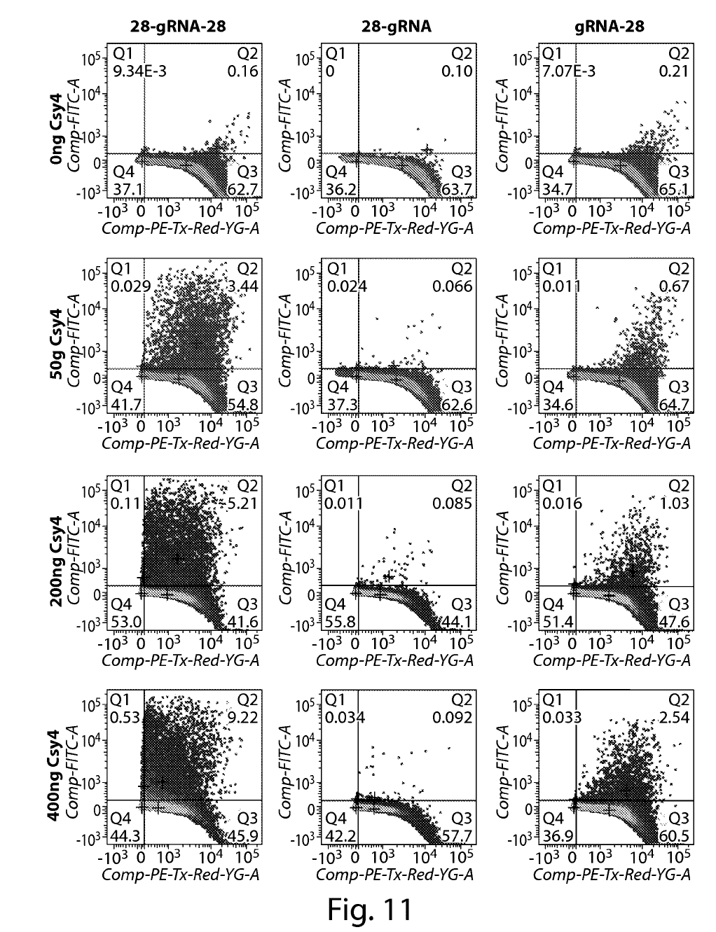

To determine whether both of the 5′ and 3′ Csy4 recognition sites are necessary for functional gRNA generation from introns, an HSV1-based intron was used within mKate2. This intron housed a gRNA1 sequence that was either preceded by a Csy4 binding site on its 5′ side (‘28-gRNA’, FIG. 2E and FIG. 11) or followed by a Csy4 binding site on its 3′ end (‘gRNA-28’, FIG. 2F and FIG. 11). The synthetic P1-EYFP construct was used to assess gRNA1 activity. The data for FIGS. 2E and 2F was normalized with the performance of the ‘intron/Csy4’ configuration where intronic gRNA1 was flanked by two Csy4 binding sites (‘28-gRNA-28’, FIG. 11). Both configurations containing only a single Csy4 binding site had mKate2 levels which decreased with the addition of Csy4 versus no Csy4 (FIGS. 2E, 2F).

In contrast, downstream EYFP activation by the gRNA1-directed CRISPR-TF was significantly lower for the single Csy4-binding-site configurations (FIGS. 2E, 2F) versus the ‘intron/Csy4’ construct (FIG. 2D). When only one Csy4 binding site was located at the 5′ end of the gRNA1 intron, EYFP expression was not detectable (FIG. 2E). When only one Csy4 binding site was located at the 3′ end of the gRNA1 intron, a 6-fold reduction in EYFP levels was observed (FIG. 2F) compared with the ‘intron/Csy4’ configuration, which contains Csy4 recognition sites flanking gRNA1 (FIG. 2D). Without being bound by theory, it is possible that Csy4 can help stabilize intronic gRNA. For example, the 5′ end of RNAs cleaved by Csy4 contain a hydroxyl (OH—) which may protect them from major 5′->3′ cellular RNases such as the XRN family, which require a 5′ phosphate for substrate recognition (Houseley and ToHervey, 2009; Nagarajan et al., 2013). In addition, binding of the Csy4 protein to the 3′ end of the cleaved gRNA (Haurwitz et al., 2012) may protect it from 3′->5′ degradation mediated by the eukaryotic exosome complex (Houseley and Tollervey, 2009).

### Example 5

### Functional gRNA Generation with Cis-Acting Ribozymes

In addition to the ‘triplex/Csy4’ and ‘intron/Csy4’-based mechanisms described above, self-cleaving ribozymes were also employed to enable gene regulation in human cells via gRNAs generated from RNAP II promoters. Specifically, the gRNAs were engineered to contain a hammerhead (HH) ribozyme (Pley et al., 1994) on their 5′ end and a HDV ribozyme (Ferre-D'Amare et al., 1998) on their 3′ end, as shown in FIG. 3. Ribozymes in three different configurations were tested, all driven by a CMVp: (1) an mKate2 transcript followed by a triplex and a HH-gRNA1-HDV sequence (CMVp-mK-Tr-HH-g1-HDV, FIG. 3A); (2) an mKate2 transcript followed a HH-gRNA1-HDV sequence (CMVp-mK-HH-g1-HDV, FIG. 3B); and (3) the sequence HH-gRNA1-HDV itself with no associated protein coding sequence (CMVp-HH-g1-HDV, FIG. 3C). gRNAs generated from these configurations were compared with gRNAs produced by the RNAP III promoter U6 and the ‘triplex/Csy4’ configuration (with 200 ng of the Csy4 plasmid) described earlier. All constructs utilized gRNA1, which drove the expression of EYFP from a P1-EYFP-containing plasmid.

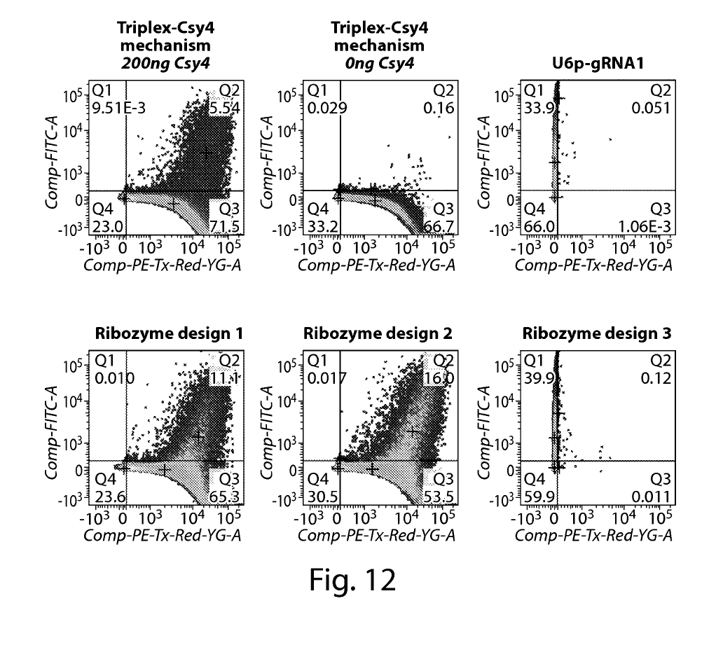

All the constructs that contained mKate2 exhibited detectable mKate2 fluorescence levels (FIG. 3D and FIG. 12). Surprisingly, this included CMVp-mK-HH-g1-HDV, which did not have a triplex sequence and was thus expected to have low mKate2 levels due to removal of the poly-(A) tail. Without being bound by theory, this could be due to inefficient ribozyme cleavage (Beck and Nassal, 1995; Chowrira et al., 1994; R Hormes, 1997), which allows non-processed transcripts to be transported to the cytoplasm and translated, protection of the mKate2 transcript by the residual 3′ ribozyme sequence, or other mechanisms. In terms of output EYFP activation, the highest EYFP fluorescence level was generated from gRNAs expressed by U6p, followed by the CMVp-HH-g1-HDV and CMVp-mK-HH-g1-HDV constructs (FIG. 3D). The CMVp-mK-Tr-HH-gRNA1-HDV and ‘triplex/Csy4’ configurations had similar EYFP levels.

Cis-acting ribozymes are useful and can mediate functional gRNA expression from RNAP II promoters. Ribozymes with activities that can be regulated with external ligands, such as theophylline, could also be used to trigger gRNA release exogenously. However, such strategies cannot link intracellular ribozyme activity to endogenous signals generated within single cells. In contrast, as shown below, the expression of genetically encoded Csy4 can be used to rewire RNA-directed genetic circuits and change their behavior (FIG. 7). Thus, trans-activating ribozymes could be used to link RNA cleavage and gRNA generation to intracellular events.

### Example 6

### Multiplexed gRNA Expression from Single RNA Transcripts

To demonstrate the expression of two independent gRNAs from a single RNA transcript to activate two independent downstream promoters, two configurations were used. In the first configuration (‘intron-triplex’), gRNA1 was encoded within an HSV1 intron flanked by two Csy4 binding sites within the coding sequence of mKate2. Further, gRNA2 enclosed by two Csy4 binding sites was encoded downstream of the mKate2-triplex sequence (FIG. 4A, CMVp-mKEX1-[28-g1-28]HSV1-mKEX2-Tr-28-g2-28). In the second configuration (‘triplex-tandem’), both gRNA1 and gRNA2 were surrounded with Csy4 binding sites and placed in tandem, downstream of the mKate2-triplex sequence (FIG. 4B, CMVp-mK-Tr-28-g1-28-g2-28). In both configurations, gRNA1 and gRNA2 targeted the synthetic promoters P1-EYFP and P2-ECFP, respectively.

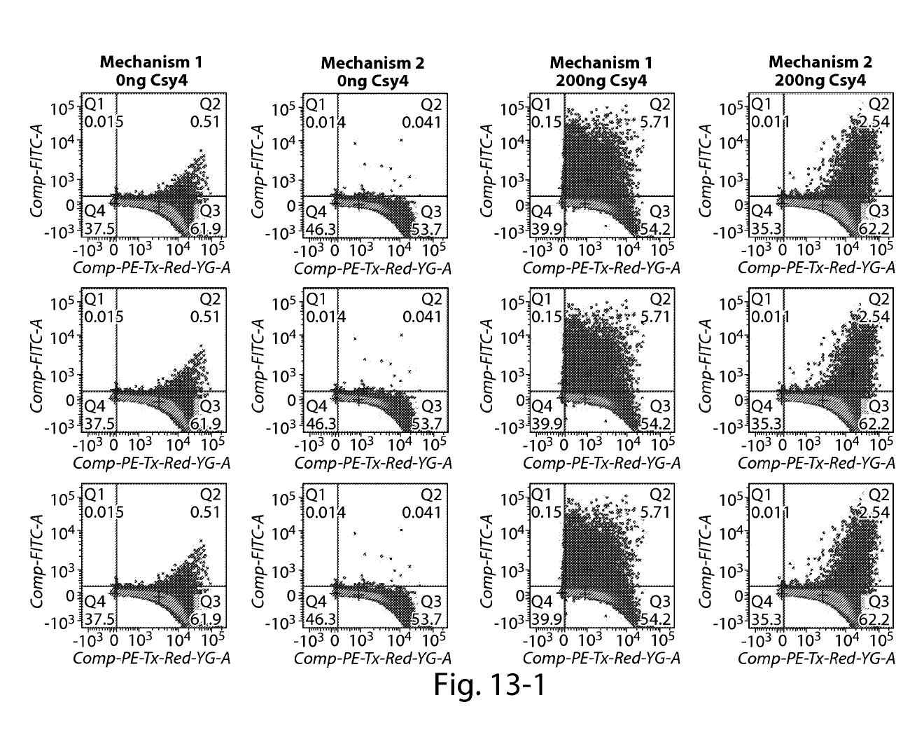

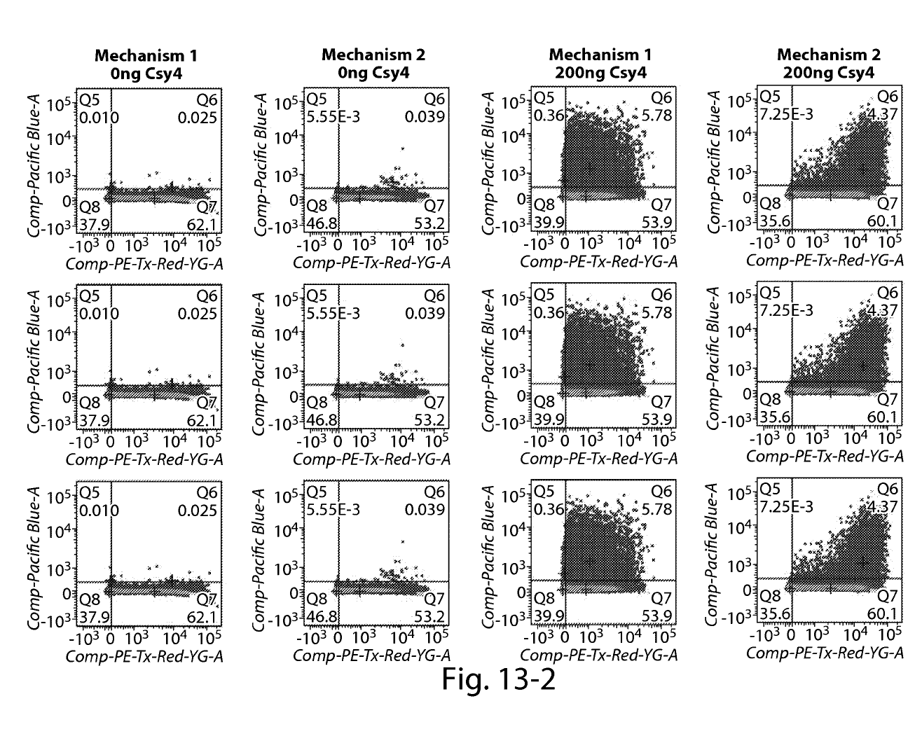

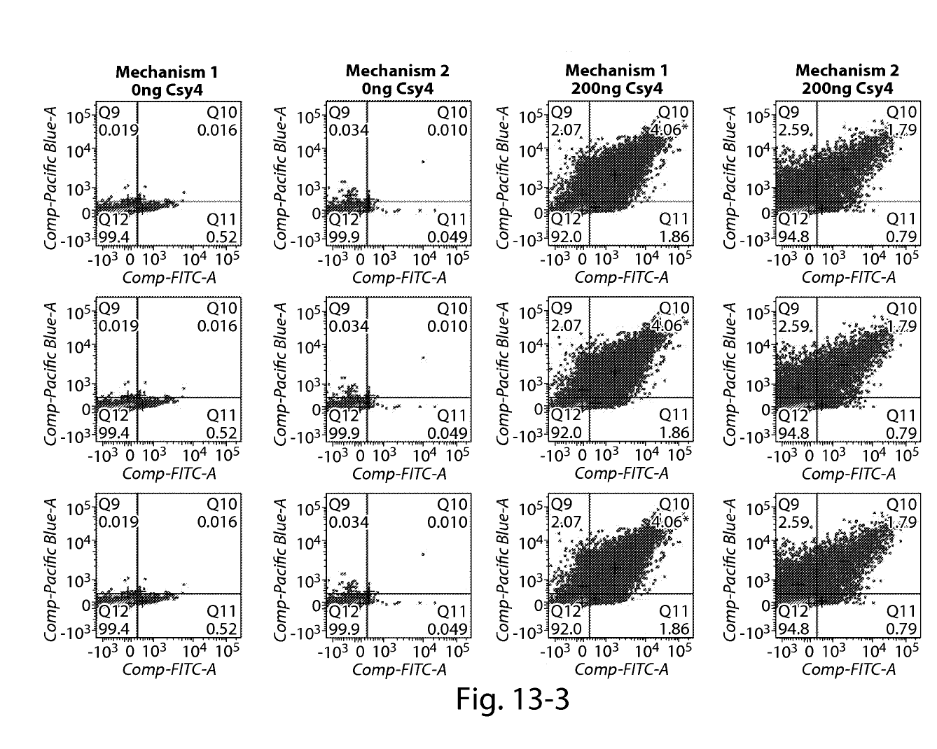

As shown in FIG. 4C (see FIG. 13 for raw data), both strategies resulted in active multiplexed gRNA production. The ‘intron-triplex’ construct exhibited a 3-fold de-crease in mKate2, a 10-fold increase in EYFP, and a 100-fold increase in ECFP in the presence of 200 ng of the Csy4 plasmid compared to no Csy4. In the ‘triplex-tandem’ configuration, mKate2, EYFP, and ECFP expression increased by 3-fold, 36-fold, and 66-fold, respectively, in the presence of 200 ng of the Csy4 plasmid compared to no Csy4. The ‘intron-triplex’ configuration had higher EYFP and ECFP levels compared with ‘triplex-tandem’ construct. Thus, both strategies for multiplexed gRNA expression enable functional CRISPR-TF activity at multiple downstream targets and can be tuned for desired applications.

To further explore the scalability of the multiplexing constructs and to demonstrate its utility in targeting endogenous loci, four different gRNAs species were generated from a single transcript. The four gRNAs required for IL1RN activation were cloned in tandem, separated by Csy4 binding sites, downstream of an mKate2-triplex sequence on a single transcript (FIG. 5A). IL1RN activation by the multiplexed single-transcript construct was compared with a configuration where the four different gRNAs were expressed from four different plasmids (FIG. 5B, ‘Multiplexed’ versus ‘Non-multiplexed’, respectively). In the presence of 100 ng of the Csy4 plasmid, the multiplexed configuration resulted in a ˜1111-fold activation over non-specific gRNA1 (‘NS’) and was ˜2.5 times more efficient than the non-multiplexed set of single-gRNA-expressing plasmids. Furthermore, ˜155-fold IL1RN activation was detected with the multiplexed configuration even in the absence of Csy4, which suggests that taCas9 can bind to gRNAs and recruit them for gene activation despite no Csy4 being present. These results demonstrate that it is possible to encode multiple functional gRNAs for multiplexed expression from a single concise RNA transcript. These configurations therefore enable compact programming of Cas9 function for implementing multi-output synthetic gene circuits, for modulating endogenous genes, and for potentially achieving conditional multiplexed genome editing.

### Example 7

### Synthetic Transcriptional Cascades with RNA-Guided Regulation

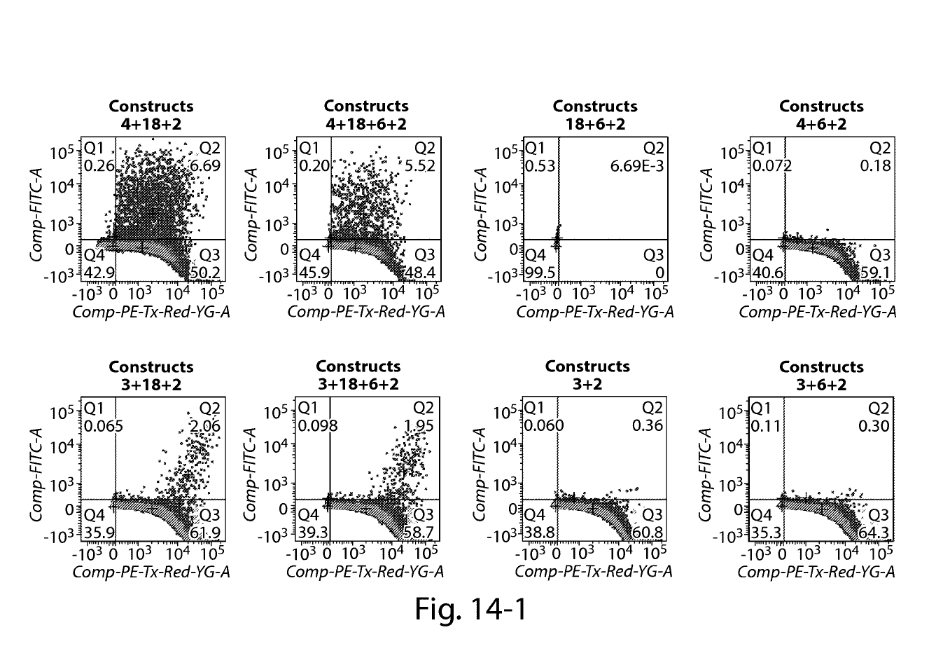

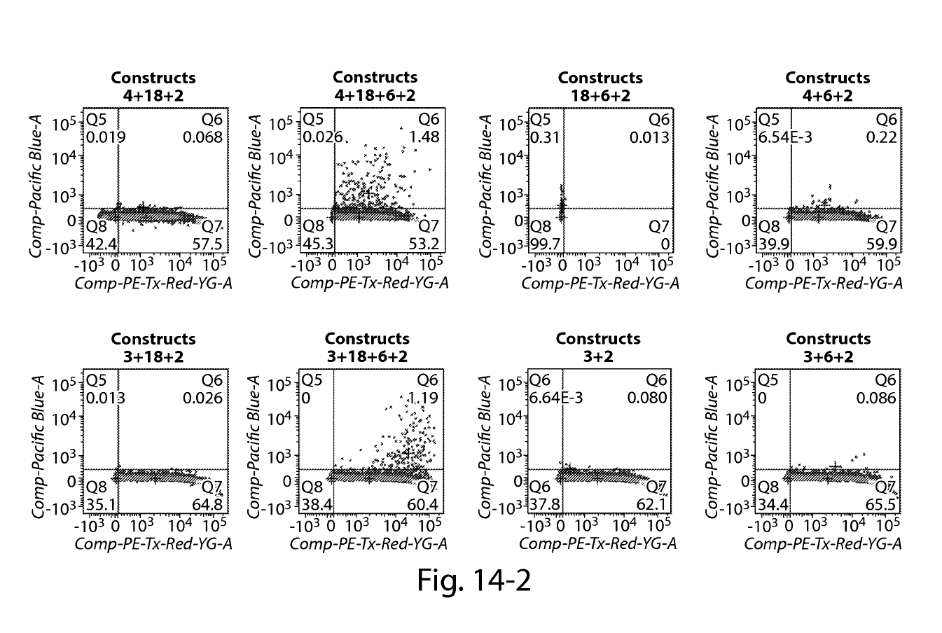

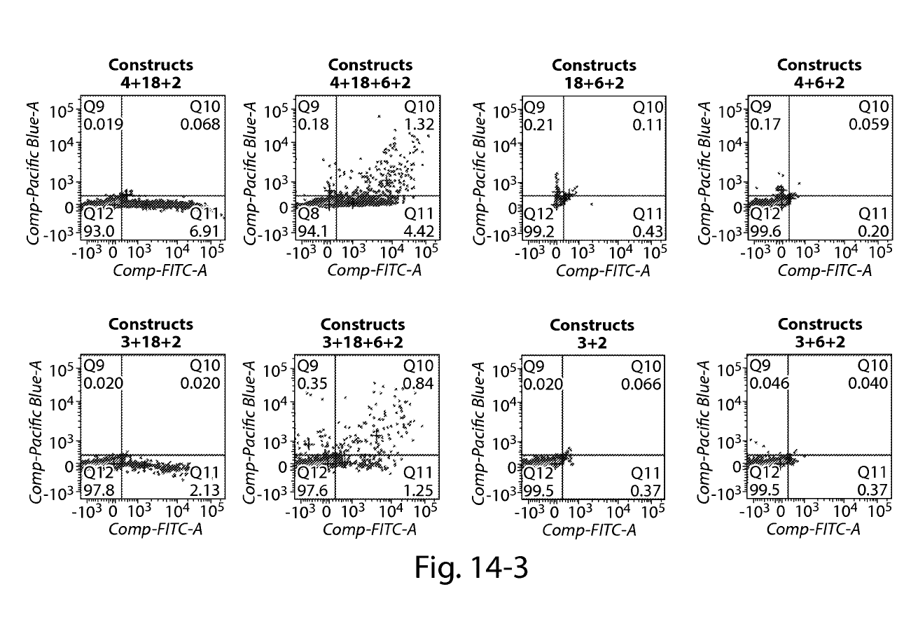

To demonstrate the utility of the RNA-dependent regulatory constructs, it was used herein to create the first CRISPR-TF-based transcriptional cascades. The ‘triplex/Csy4’ and ‘intron/Csy4’ strategies were integrated to build two different three-stage CRISPR-TF-mediated transcriptional cascades (FIG. 6). In the first design, CMVp-driven expression of gRNA1 from an ‘intron/Csy4’ construct generated gRNA1 from an HSV1 intron, which activated a synthetic promoter P1 to produce gRNA2 from a ‘triplex/Csy4’ configuration, which then activated a downstream synthetic promoter P2 regulating ECFP (FIG. 6A). In the second design, the intronic gRNA expression cassette in the first stage of the cascade was replaced by a ‘triplex/Csy4’ configuration for expressing gRNA1 (FIG. 6B). These two designs were tested in the presence of 200 ng of the Csy4 plasmid (FIGS. 6C, 6D and FIG. 14).

In the first cascade design, a 76-fold increase in EYFP and a 13-fold increase in ECFP were observed compared to a control in which the second stage of the cascade (P1-EYFP-Tr-28-g2-28) was replaced by an empty plasmid (FIG. 6C). In the second cascade design, a 31-fold increase in EYFP and a 21-fold increase in ECFP were observed compared to a control in which the second stage of the cascade (P1-EYFP-Tr-28-g2-28) was replaced by an empty plasmid (FIG. 6D). These results demonstrate that there is minimal non-specific activation of promoter P2 by gRNA1, which is essential for the scalability and reliability of transcriptional cascades. Furthermore, the fold-activation of each stage in the cascade was dependent on the presence of all upstream nodes, which is expected in properly functioning transcriptional cascades (FIGS. 6C, 6D).

### Example 8

### Rewiring RNA-Dependent Synthetic Regulatory Circuits

The following experiments sought to demonstrate how CRISPR-TF regulation can be integrated with mammalian RNA interference to implement more sophisticated circuit topologies. Furthermore, the following experiments showed how network motifs could be rewired based on Csy4-based RNA processing. Specifically, miRNA regulation was incorporated with CRISPR-TFs and used Csy4 to disrupt miRNA inhibition of target RNAs by removing cognate miRNA binding sites. A single RNA transcript was built, which was capable of expressing both a functional miRNA (Greber et al., 2008; Xie et al., 2011) and a functional gRNA. This was achieved by encoding a mammalian miRNA inside the consensus intron within the mKate2 gene, followed by a triplex sequence and a gRNA1 sequence flanked by Csy4 recognition sites (FIG. 7A, CMVp-mKEx1-[miR]-mKEx2-Tr-28-g1-28). Two output constructs were also implemented to demonstrate the potential for multiplexed gene regulation with the engineered constructs. The first output was a constitutively expressed ECFP gene followed by a triplex sequence, a Csy4 recognition site, 8× miRNA binding sites (8× miRNA-BS), and another Csy4 recognition site (FIG. 7A). The second output was a synthetic P1 promoter regulating EYFP expression (FIG. 7A).

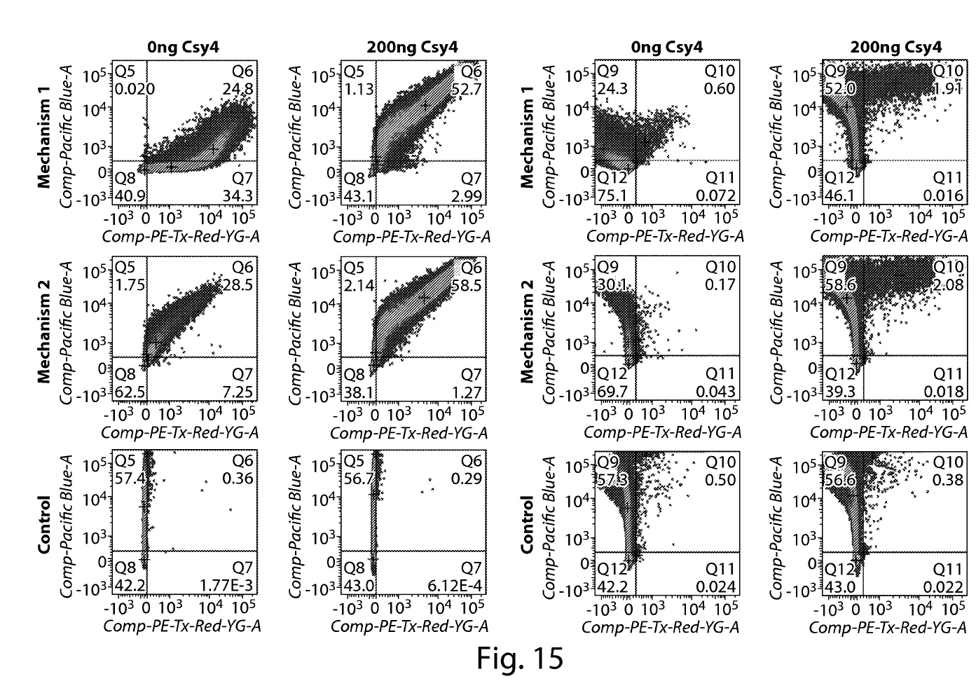

In the absence of Csy4, ECFP and EYFP levels were low because the miRNA suppressed ECFP expression and no functional gRNA1 was generated (FIG. 7B and FIG. 15 ‘Mechanism 1’). In the presence of Csy4, ECFP expression increased by 30-fold compared to the no Csy4 condition, which we attributed to Csy4-induced separation of the 8× miRNA-BS from the ECFP transcript (FIG. 7B). Furthermore, the presence of Csy4 generated functional gRNA1, leading to 17-fold increased EYFP expression compared to the no Csy4 condition (FIG. 7B). The mKate2 fluorescence levels were high in both the Csy4-positive and Csy4-negative conditions. Thus, Csy4 catalyzed RNA-based rewiring of circuit connections between the input node and its two outputs by simultaneously inactivating a repressive output link and enabling an activating output link (FIG. 7C).

To demonstrate the facile nature by which additional circuit topologies can be programmed using RNA-dependent mechanisms, the design in FIG. 7A was extended by incorporating an additional 4× miRNA-BS at the 3′ end of the mKate-containing transcript (FIG. 7D, CMVp-mKEx1-[miR]-mKEx2-Tr-28-g1-28-miR4×BS). In the absence of Csy4, this resulted in autoregulatory negative-feedback suppression of mKate2 expression by the miRNA generated within the mKate2 intron (FIG. 7E and FIG. 15 ‘Mechanism 2’). In addition, both ECFP and EYFP levels remained low due to repression of ECFP by the miRNA and the lack of functional gRNA1 generation. However, in the presence of Csy4, mKate2 levels increased by 21-fold due to Csy4-mediated separation of the 4× miRNA-BS from the mKate2 transcript. Furthermore, ECFP inhibition by the miRNA was relieved in a similar fashion, resulting in a 27-fold increase in ECFP levels. Finally, functional gRNA1 was generated, leading to a 50-fold increase in EYFP levels (FIG. 7E). Thus, Csy4 catalyzed RNA-based rewiring of circuit connections between the input node and its two outputs by simultaneously inactivating a repressive output link, enabling an activating output link, and inactivating an autoregulatory feed-back loop (FIG. 7F).

Synthetic biology provides tools for studying natural regulatory networks by disrupting, rewiring, and mimicking natural network motifs. In addition, synthetic circuits can used to link exogenous signals to endogenous gene regulation to address biomedical applications and to perform cellular computation. Although many synthetic gene circuits are based on transcriptional regulation, RNA-based regulation can be used to construct a variety of synthetic gene circuits. Despite many advances, previous efforts have not yet integrated RNA-based regulation with CRISPR-TFs, which are both promising strategies for implementing scalable genetic circuits given their programmability and potential for multiplexing. Provided herein are constructs for engineering artificial gene circuits and endogenous gene regulation in human cells. This framework integrates mammalian RNA regulatory mechanisms with the RNA-dependent protein, dCas9, and the RNA-processing protein, Csy4, from bacteria. Moreover, it enables convenient programming of regulatory links based on base-pairing complementary between nucleic acids.

Provided herein, in some embodiments, are multiple complementary approaches to generate functional gRNAs from the coding sequence of proteins regulated by RNAP II promoters, which also permit concomitant expression of the protein of interest. The genes used were fluorescent genes because they are convenient reporters of promoter activity. However, these genes can be readily exchanged with any other protein-coding sequence, thus enabling multiplexed expression of gRNAs along with arbitrary protein outputs from a single construct. The ability of these strategies was validated, based on RNA triplexes with Csy4, RNA introns with Csy4, and cis-acting ribozymes, to generate functional gRNAs by targeting synthetic promoters. Furthermore, when gRNAs were flanked by Csy4 recognition sites and located downstream of a gene followed by an RNA triplex, the levels of the gene increased with the levels of Csy4. The opposite effect was found when gRNAs were flanked by Csy4 recognition sites within introns, with the magnitude of the effect varying depending on the specific intronic sequence used. Thus, these complementary configurations enable tunable RNA and protein levels to be achieved within synthetic gene circuits.

As a complement to synthetic circuits, engineered constructs of the present disclosure can be used, in some embodiments, to activate endogenous promoters from multiple different human RNAP II promoters, as well as the CMV promoter. Provided herein, in some embodiments, are novel strategies for multiplexed gRNA expression from compact single transcripts to modulate both synthetic and native promoters. This feature is useful because, for example, it can be used to regulate multiple nodes from a single one. The ability to concisely encode multiple gRNAs within a single transcript enables sophisticated circuits with a large number of parallel ‘fan-outs’ (e.g., outgoing interconnections from a given node) and networks with dense interconnections. Moreover, the ability to synergistically modulate endogenous loci with several gRNAs in a condensed fashion is advantageous, for example, because multiple gRNAs are often needed to enact substantial modulation of native promoters. Thus, the engineered constructs described herein can be used, in some instances, to build efficient artificial gene networks and to perturb native regulatory networks.

In addition to transcriptional regulation, a nuclease-proficient Cas9 may be used instead of taCas9, in some embodiments, to conditionally link multiplexed genome-editing activity to cellular signals via regulation of gRNA expression. This enables conditional, multiplexed knockouts within in vivo settings—for example, with cell-specific, temporal, or spatial control. In addition to genetic studies, this capability can be used, in some embodiments, to create in vivo DNA-based ‘ticker tapes’ that link cellular events to mutations.

These configurations lay down a foundation, in some embodiments, for the construction of sophisticated and compact synthetic gene circuits in human cells. Without being bound by theory, because the specificity of regulatory interconnections with the engineered constructs is determined only by RNA sequences, scalable circuits with almost any network topology can be constructed. For example, multi-layer network topologies are important for achieving sophisticated behaviors, both in artificial and natural genetic contexts. Thus, to demonstrate the utility of the present constructs for implementing more complex synthetic circuits, they was used to create the first CRISPR-TF-based transcriptional cascades which were highly specific and effective. Demonstrated by the examples provided herein are reliable three-step transcriptional cascades with two different configurations that incorporated RNA triplexes, introns, Csy4 and CRISPR-TFs. The absence of undesired crosstalk between different stages of the cascade underscores the orthogonality and scalability of RNA-dependent regulatory schemes for synthetic gene circuit design. Combining multiplexed gRNA expression with transcriptional cascades can be used, in some instances to create multi-stage, multi-input/multi-output gene networks capable of logic, computing, and interfacing with endogenous systems. In addition, useful topologies, such as multi-stage feedforward and feedback loops, can be readily programmed, in some embodiments.

Furthermore, RNA regulatory parts, such as CRISPR-TFs and RNA interference, were integrated together to create various circuit topologies that can be rewired via conditional RNA processing. Because both positive and negative regulation is possible with the same taCas9 protein and miRNAs enact tunable negative regulation, many important multi-component network topologies can be implemented using this set of regulatory parts. In addition, Csy4 can be used, for example, to catalyze changes in gene expression by modifying RNA transcripts. For example, functional gRNAs were liberated for transcriptional modulation and miRNA binding sites were removed from RNA transcripts to eliminate miRNA-based links. In addition, the absence or presence of Csy4 was used to switch a miRNA-based autoregulatory negative feedback loop on and off, respectively (FIG. 7B). This feature, in some embodiments, can be extended in circuits to minimize unwanted leakage in positive-feedback loops and to dynamically switch circuits between different states. By linking Csy4 expression, for example, to endogenous promoters, interconnections between circuits and network behavior could also be conditionally linked to specific tissues, events (e.g., cell cycle phase, mutations), or environmental conditions. With genome mining or directed mutagenesis on Csy4, orthogonal Csy4 variants can used for more complicated RNA processing schemes. Moreover, additional flexibility and scalability can be achieved by using orthogonal Cas9 proteins.

In summary, the present disclosure provides a diverse set of constructs for building scalable regulatory gene circuits, tuning them, modifying connections between circuit components, and synchronizing the expression of multiple genes in a network. Furthermore, these regulatory parts can be used, in some embodiments, to interface synthetic gene circuits with endogenous systems as well as to rewire endogenous networks. Integrating RNA-dependent regulatory mechanisms with RNA processing will enable sophisticated transcriptional and post-transcriptional regulation, accelerate synthetic biology, and facilitate the study of basic biology in human cells.

### Plasmid Construction

The CMVp-dCas9-3×NLS-VP64 (taCas9, Construct 1, Table 2) plasmid was built as described previously (Farzadfard et al., 2013). The csy4 gene from Pseudomonas aeruginosa strain UCBPP-PA14 (Qi et al., 2012), was codon optimized for expression in human cells, PCR amplified to contain an N-terminal 6x-His tag and a TEV recognition sequence, and cloned downstream of the PGK1 promoter between HindIII/SacI sites in the PGK1-EBFP2 plasmid (Farzadfard et al., 2013) to create PGK1p-Csy4-pA (Construct 2, Table 2).

TABLE 2
Construct names, designs, and abbreviations

| | |
|---|---|
| Construct 1 | CMVp-dCas9-3xNLS-VP64-3′LTR |
| Abbreviation | taCas9 |
| Construct 2 | PGK1p-Csy4-pA |
| Abbreviation | Csy4 |
| Construct 3 | CMVp-mKate2-Triplex-28-gRNA1-28-pA |
| Abbreviation | CMVp-mK-Tr-28-g1-28 |
| Construct 4 | CMVp-mKate2_EX1-[28-gRNA1-28]_{HSV1}-mKate2_EX2-pA |
| Abbreviation | CMVp-mK_{EX1}-[28-g1-28]_{HSV1}-mK_{EX2} |
| Construct 5 | P1-EYFP-pA |
| Abbreviation | P1-EYFP |
| Construct 6 | P2-ECFP-pA |
| Abbreviation | P2-ECFP |
| Construct 7 | U6p-gRNA1-TTTTT |
| Abbreviation | U6p-g1 |
| Construct 8 | CMVp-mKate2_EX1-[28-gRNA1-28]_{consensus}-mKate2_EX2-pA |
| Abbreviation | CMVp-mK_{EX1}-[28-g1-28]_{cons}-mK_{EX2} |
| Construct 9 | CMVp-mKate2_EX1-[28-gRNA1-28]_{snoRNA2}-mKate2_EX2-pA |
| Abbreviation | CMVp-mK_{Ex1}-[28-g1-28]ₛₙₒ-mK_{Ex2} |
| Construct 10 | CMVp-mKate2_EX1-[28-gRNA1]_{HSV1}-mKate2_EX2-pA |
| Abbreviation | CMVp-mK_{EX1}-[28-g1]_{HSV1}-mK_{EX2} |
| Construct 11 | CMVp-mKate2_EX1-[gRNA1-28]_{HSV1}-mKate2_EX2-pA |
| Abbreviation | CMVp-mK_{EX1}-[g1-28]_{HSV1}-mK_{EX2} |
| Construct 12 | CMVp-mKate2_exon1-[gRNA1]_{consensus}-mKate2_EX2-pA |
| Abbreviation | CMVp-mK_{EX1}-[g1]_{cons}-mK_{EX2} |
| Construct 13 | CMVp-mKate2-Triplex-HHRibo-gRNA1-HDVRibo-pA |
| Abbreviation | CMVp-mK-Tr-HH-g1-HDV |
| Construct 14 | CMVp-mKate2-HHRibo-gRNA1-HDVRibo-pA |
| Abbreviation | CMVp-mK-HH-g1-HDV |
| Construct 15 | CMVp-HHRibo-gRNA1-HDVRibo-pA |
| Abbreviation | CMVp-HH-g1-HDV |
| Construct 16 | CMVp-mKate2_EX1-[28-gRNA1-28]_{HSV1}-mKate2_EX2-Triplex-28- |
| | gRNA2-28-pA |
| Abbreviation | CMVp-mK_{EX1}-[28-g1-28]_{HSV1}-mK_{EX2}-Tr-28-g2-28 |
| Construct 17 | CMVp-mKate2-Triplex-28-gRNA1-28-gRNA2-28-pA |
| Abbreviation | CMVp-mK-Tr-28-g1-28-g2-28 |
| Construct 18 | P1-EYFP-Triplex-28-gRNA2-28-pA |
| Abbreviation | P1-EYFP-Tr-28-g2-28 |
| Construct 19 | CMVp-mKate2-Triplex-28-gRNA3-28-gRNA4-28-gRNA5-28-gRNA6- |
| | 28-pA |
| Abbreviation | CMVp-mK-Tr-(28-g-28)₃₋₆ |
| Construct 20 | CMVp-mKate2_EX1-[miRNA]-mKate2_EX2-Triplex-28-gRNA1-28-pA |
| Abbreviation | CMVp-mK_{EX1}-[miR]-mK_{EX2}-Tr-28-g1-28 |
| Construct 21 | CMVp-mKate2_EX1-[miRNA]-mKate2_EX2-Triplex-28-gRNA1-28- |
| | 4xFF4BS-pA |
| Abbreviation | CMVp-mK_{EX1}-[miR]-mK_{EX2}-Tr-28-g1-28-miR_{4xBS} |
| Construct 22 | CMVp-ECFP-Triplex-28-8xmiRNA-BS-28-pA |
| Abbreviation | CMVp-ECFP-Tr-28-miR_{8xBS}-28 |
| Construct 23 | CMVp-mKate2_Triplex-28-gRNA3-28 |
| Abbreviation | CMVp-mK-28-Tr-28-g3-28 |
| Construct 24 | CMVp-mKate2_Triplex-28-gRNA4-28 |
| Abbreviation | CMVp-mK-28-Tr-28-g4-28 |
| Construct 25 | CMVp-mKate2_Triplex-28-gRNA5-28 |
| Abbreviation | CMVp-mK-28-Tr-28-g5-28 |
| Construct 26 | CMVp-mKate2_Triplex-28-gRNA6-28 |
| Abbreviation | CMVp-mK-28-Tr-28-g6-28 |

The plasmid CMVp-mKate2-Triplex-28-gRNA1-28-pA (Construct 3, Table 2) was built using GIBSON ASSEMBLY® from three parts amplified with appropriate homology overhangs: 1) the full length coding sequence of mKate2; 2) the first 110 base pair (bp) of the mouse MALAT1 3′ triple helix (Wilusz et al., 2012); and 3) gRNA1 containing a 20 bp Specificity Determining Sequence (SDS) and a S. pyogenes gRNA scaffold along with 28 nucleotide (nt) Csy4 recognition sites.

The reporter plasmids P1-EFYP-pA (Construct 5, Table 2) and P2-ECFP-pA (Construct 6, Table 2) were built by cloning in eight repeats of gRNA1 binding sites and eight repeats of gRNA2 binding sites into the NheI site of pG5-Luc (Promega) via annealing complementary oligonucleotides. Then, EYFP and ECFP were cloned into the NcoI/FseI sites, respectively.

The plasmid CMVp-mKate2_EX1-[28-gRNA1-28]_{HSV1}-mKate2_EX2-pA (Construct 4, Table 2) was built by GIBSON ASSEMBLY® of the following parts with appropriate homology overhangs: 1) the mKate2_EX1 (a.a. 1-90) of mKate2; 2) mKate_EX2 (a.a. 91-239) of mKate2; and 3) gRNA1 containing a 20 bp SDS followed by the S. pyogenes gRNA scaffold flanked by Csy4 recognition sites and the HSV1 acceptor, donor and branching sequences. Variations of the CMVp-mKate2_EX1-[28-gRNA1-28]_{HSV1}-mKate2_EX2-pA plasmid containing consensus and SnoRNA2 acceptor, donor, and branching sequences and with and without the Csy4 recognition sequences (Constructs 8-12, Table 2) were built in a similar fashion.

The ribozyme-expressing plasmids CMVp-mKate2-Triplex-HHRibo-gRNA1-HDVRibo-pA and CMVp-mKate2-HHRibo-gRNA1-HDVRibo-pA plasmids (Constructs 13 and 14, respectively, Table 2) were built by GIBSON ASSEMBLY® of XmaI-digested CMVp-mKate2, and PCR-extended amplicons of gRNA1 (with and without the triplex and containing HHRibo (Gao and Zhao, 2014) on the 5′ end and HDVRibo (Gao and Zhao, 2014) on the 3′ end). The plasmid CMVp-HHRibo-gRNA1-HDVRibo-pA (Construct 15, Table 2) was built similarly by GIBSON ASSEMBLY® of SacI-digested CMVp-mKate2 and a PCR-extended amplicon of gRNA1 containing HHRibo on the 5′ end and HDVRibo on the 3′ end. The plasmid CMVp-mKate2_EX1-[28-gRNA1-28]_{HSV1}-mKate2_EX2-Triplex-28-gRNA2-28-pA (Construct 16, Table 2) was built by GIBSON ASSEMBLY® of the following parts using appropriate homologies: 1) XmaI-digested CMVp-mKate2_EX1-[28-gRNA1-28]_{HSV1}-mKate2_EX2-pA (Construct 4, Table 2) and 2) PCR amplified Triplex-28-gRNA2-28 from CMVp-mKate2-Triplex-28-gRNA1-28-pA (Construct 3, Table 2).

The plasmid CMVp-mKate2-Triplex-28-gRNA1-28-gRNA2-28-pA (Construct 17, Table 2) was built by GIBSON ASSEMBLY® with the following parts using appropriate homologies: 1) XmaI-digested CMVp-mKate2-Triplex-28-gRNA1-28-pA (Construct 3, Table 2) and 2) PCR amplified 28-gRNA2-28.

The plasmid CMVp-mKate2-Triplex-28-gRNA3-28-gRNA4-28-gRNA5-28-gRNA6-28-pA (Construct 19, Table 2) was constructed using a Golden Gate approach using the Type IIs restriction enzyme, BsaI. Specifically, the IL1RN targeting gRNA3, gRNA4, gRNA5, gRNA6 sequences containing the 20 bp SDSs along with the S. pyogenes gRNA scaffold were PCR amplified to contain a BsaI restriction site on their 5′ ends and Csy4 ‘28’ and BsaI restriction sites on their 3′ ends. The PCR amplified products were subjected to 30 alternating cycles of digestion followed by ligation at 37° C. and 20° C., respectively. A 540 bp PCR product containing the gRNA3-28-gRNA4-28-gRNA5-28-gRNA6-28 array was amplified and digested with NheI/XmaI and cloned into the CMVp-mKate2-Triplex-28-gRNA1-28-pA plasmid (Construct 3, Table 2).

The CMVp-mKate2_EX1-[miRNA]-mKate2_EX2-pA plasmid containing an intronic FF4 (a synthetic miRNA) was received as a gift from Lila Wroblewska. The synthetic FF4 miRNA was cloned into an intron with consensus acceptor, donor and branching sequences between a.a. 90 and 91 of mKate2 to create CMVp-mKate2_EX1-[miRNA]-mKate2_EX2-Triplex-28-gRNA1-28-pA (Construct 20, Table 2) and CMVp-mKate2_EX1-[miRNA]-mKate2_EX2-Triplex-28-gRNA1-28-4×FF4BS-pA (Construct 21, Table 2).

The plasmid CMVp-ECFP-Triplex-28-8×miRNA-BS-28-pA (Construct 22, Table 2) was cloned via GIBSON ASSEMBLY® with the following parts: 1) full length coding sequence of ECFP and 2) 110 nt of the MALAT1 3′ triple helix sequence amplified via PCR extension with oligonucleotides containing eight FF4 miRNA binding sites and Csy4 recognition sequences on both ends.

### Cell Culture and Transfections

HEK293T cells were obtained from the American Tissue Collection Center (ATCC) and were maintained in Dulbecco's Modified Eagle Medium (DMEM) supplemented with 10% fetal bovine serum (FBS), 1% penicillin-streptomycin, 1% GlutaMAX, non-essential amino acids at 37° C. with 5% CO₂. HEK293T cells were transfected with FuGENE®HD Transfection Reagent (Promega) according to the manufacturer's instructions. Each transfection was made using 200,000 cells/well in a 6-well plate. As a control, with 2 μg of a single plasmid in which a CMV promoter regulated mKate2, transfection efficiencies were routinely higher than 90% (determined by flow cytometry performed with the same settings as the experiments). Unless otherwise indicated, each plasmid was transfected at 1 μg/sample. All samples were transfected with taCas9, unless specifically indicated. Cells were processed for flow cytometry or qRT-PCR analysis 72 hours after transfection.

### Quantitative Reverse Transcription—PCR (RT-PCR)

The experimental procedure followed was as described in (Perez-Pinera et al., 2013a). Cells were harvested 72 hour post-transfection. Total RNA was isolated using the RNeasy Plus RNA isolation kit (Qiagen). cDNA synthesis was performed using the qScript cDNA SuperMix (Quanta Biosciences). Real-time PCR using PerfeCTa SYBR Green FastMix (Quanta Biosciences) was performed with the Mastercycler ep realplex real-time PCR system (Eppendorf) with following oligonucleotide primers: IL1RN—forward GGAATCCATGGAGGGAAGAT (SEQ ID NO: 22), reverse TGTTCTCGCTCAGGTCAGTG (SEQ ID NO: 23); GAPDH—forward CAATGACCCCTTCATTGACC (SEQ ID NO: 24), reverse TTGATTTTGGAGGGATCTCG (SEQ ID NO: 25). The primers were designed using Primer3Plus software and purchased from IDT. Primer specificity was confirmed by melting curve analysis. Reaction efficiencies over the appropriate dynamic range were calculated to ensure linearity of the standard curve. Fold-increases in the mRNA expression of the gene of interest normalized to GAPDH expression were calculated by the ddCt method. We then normalized the mRNA levels to the non-specific gRNA1 control condition. Reported values are the means of three independent biological replicates with technical duplicates that were averaged for each experiment. Error bars represent standard error of the mean (s.e.m).

### Flow Cytometry

Cells were harvested with trypsin 72 hours post-transfection, washed with DMEM media and 1×PBS, re-suspended with 1×PBS into flow cytometry tubes and immediately assayed with a Becton Dickinson LSRII Fortessa flow cytometer. At least 50,000 cells were recorded per sample in each data set. The results of each experiment represent data from at least three biological replicates. Error bars are s.e.m. on the weighted median fluorescence values (see Extended Experimental Procedures for detailed information about data analysis).

### Compensation Controls

Compensation controls were strict and designed to remove false-positive cells even at the cost of removing true-positive cells. Compensation was done with BD FACSDiva (version no. 6.1.3; BD Biosciences) as detailed below:

TABLE 3
Compensation setup for flow cytometry

| Fluorochrome | -% Fluorochrome | Spectral Overlap |
|---|---|---|
| PE-Tx-Red-YG | FITC | 0% |
| Pacific Blue | FITC | 0.2% |
| FITC | PE-Tx-Red-YG | 21.1% |
| Pacific Blue | PE-Tx-Red-YG | 1% |
| FITC | Pacific Blue | 7.5% |

### Flow Cytometry Analysis

Compensated flow cytometry results were analyzed using FlowJo software (vX.0.7r2). Calculations were performed as described below:

All samples were gated to exclude cell clumps and debris (population P1) Histograms of P1 cells were analyzed according to the following gates, which were determined according to the auto-fluorescence of non-transfected cells in the same acquisition conditions such that the proportion of false-positive cells would be lower than 0.1%:

mKate2: ‘mKate2 positive’ cells were defined as cells above a fluorescence threshold of 100 a.u.

EYFP: ‘EYFP positive’ cells were defined as cells above a fluorescence threshold of 300 a.u.

ECFP: ‘ECFP positive’ cells were defined as cells above a fluorescence threshold of 400 a.u.

The percent of positive cells (% positive) and the median fluorescence for each ‘positive cell’ population were calculated. The % positive cells was multiplied by the median fluorescence, resulting in a weighted median fluorescence expression level that correlated fluorescence intensity with cell numbers. This measurement strategy is consistent with several previous studies (Auslander et al., 2012; Xie et al., 2011).

The weighted median fluorescence was determined for each sample. The mean of the weighted median fluorescence of biological triplicates was calculated. These are the data presented in the paper. The standard error of the mean (s.e.m.) was also computed and presented as error bars.

To facilitate comparisons between various constructs and to account for variations in the brightness of different fluorescent proteins, the weighted median fluorescence for each experimental condition was divided by the maximum weighted median fluorescence for the same fluorophore among all conditions tested in the same set of experiments.

Flow cytometry data plots shown in the Supplemental information are representative compensated data from a single experiment. As noted above, cells were gated to exclude cell clumps and debris (population P1), and the entire gated population of viable cells are presented in each figure. The threshold for each sub-population Q1-Q4 was set according to the thresholds described above. The percentage of cells in each sub-population is indicated in the plots. Black crosses in the plots indicate the median fluorescence for a specific sub-population.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

All references, patents and patent applications disclosed herein are incorporated by reference with respect to the subject matter for which each is cited, which in some cases may encompass the entirety of the document.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### REFERENCES

(Each reference below is incorporated by reference herein.)
Alon, U. (2007). Network motifs: theory and experimental approaches. Nat Rev Genet 8, 450-461.
Audibert, A., Weil, D., and Dautry, F. (2002). In vivo kinetics of mRNA splicing and transport in mammalian cells. Mol Cell Biol 22, 6706-6718.
Auslander, S., Auslander, D., Muller, M., Wieland, M., and Fussenegger, M. (2012). Programmable single-cell mammalian biocomputers. Nature 487, 123-127.
Babiskin, A. H., and Smolke, C. D. (2011). A synthetic library of RNA control modules for predictable tuning of gene expression in yeast. Molecular systems biology 7, 471.
Barrangou, R., and van der Oost, J. (2013). RNA-mediated Adaptive Immunity in Bacteria and Archaea (Springer).
Bashor, C. J., Helman, N. C., Yan, S., and Lim, W. A. (2008). Using engineered scaffold interactions to reshape MAP kinase pathway signaling dynamics. Science 319, 1539-1543.
Beck, J., and Nassal, M. (1995). Efficient hammerhead ribozyme-mediated cleavage of the structured hepatitis B virus encapsidation signal in vitro and in cell extracts, but not in intact cells. Nucleic Acids Research 23, 4954-4962.
Beerli, R. R., and Barbas, C. F., 3rd (2002). Engineering polydactyl zinc-finger transcriptionfactors. Nat Biotechnol 20, 135-141. Benenson, Y. (2012). Biomolecular computing systems: principles, progress and potential. NatRev Genet 13, 455-468.
Block, T M., and Hill, J. M. (1997). The latency associated transcripts (LAT) of herpes simplexvirus: still no end in sight. J Neurovirol 3, 313-321.
Blount, B. A., Weenink, T., Vasylechko, S., and Ellis, T. (2012). Rational diversification of a promoter providing fine-tuned expression and orthogonal regulation for synthetic biology. PLoSOne 7, e33279.
Chen, M., and Manley, J. L. (2009). Mechanisms of alternative splicing regulation: insights from molecular and genomics approaches. Nature reviews Molecular cell biology 10, 741-754.
Chen, Y. Y., Jensen, M. C., and Smolke, C. D. (2010). Genetic control of mammalian T-cell proliferation with synthetic RNA regulatory systems. Proceedings of the National Academy of Sciences of the United States of America 107, 8531-8536.
Cheng, A. W., Wang, H., Yang, H., Shi, L., Katz, Y., Theunissen, T. W., Rangarajan, S., Shivalila, C. S., Dadon, D. B., and Jaenisch, R. (2013). Multiplexed activation of endogenous genes by CRISPR-on, an RNA-guided transcriptional activator system. Cell Res 23, 1163-1171.
Chowrira, B. M., Pavco, P. A., and McSwiggen, I A. (1994). In vitro and in vivo comparison of hammerhead, hairpin, and hepatitis delta virus self-processing ribozyme cassettes. Journal of Biological Chemistry 269, 25856-25864.
Clement, J. Q., Qian, L., Kaplinsky, N., and Wilkinson, M. F. (1999). The stability and fate of a spliced intron from vertebrate cells. Rna 5, 206-220.
Cong, L., Ran, F. A., Cox, D., Lin, S., Barretto, R., Habib, N., Hsu, P. D., Wu, X., Jiang, W., Marraffini, L. A., et al. (2013). Multiplex Genome Engineering Using CRISPR/Cas Systems. Science 339, 819-823.
Cronin, C. A., Gluba, W., and Scrable, H. (2001). The lac operator-repressor system is functional in the mouse. Genes & development 15, 1506-1517.
Culler, S. J., Hoff, K. G., and Smolke, C. D. (2010). Reprogramming cellular behavior with RNA controllers responsive to endogenous proteins. Science 330, 1251-1255.
Deans, T. L., Cantor, C. R., and Collins, J. J. (2007). A tunable genetic switch based on RNAi and repressor proteins for regulating gene expression in mammalian cells. Cell 130, 363-372.
Delebecque, C. J., Lindner, A. B., Silver, P. A., and Aldaye, F. A. (2011). Organization of intracellular reactions with rationally designed RNA assemblies. Science 333, 470-474.
Dequeant, M. L., and Pourquie, O. (2008). Segmental patterning of the vertebrate embryonic axis. Nat Rev Genet 9, 370-382.
Ellefson, J. W., Meyer, A. J., Hughes, R. A., Cannon, J. R., Brodbelt, J. S., and Ellington, A. D. (2014). Directed evolution of genetic parts and circuits by compartmentalized partnered replication. Nat Biotech 32, 97-101.
Ellis, T., Wang, X., and Collins, J. J. (2009). Diversity-based, model-guided construction of synthetic gene networks with predicted functions. Nat Biotechnol 27, 465-471.
Elowitz, M., and Lim, W. A. (2010). Build life to understand it. Nature 468, 889-890.
Esvelt, K. M., Carlson, J. C., and Liu, D. R. (2011). A system for the continuous directed evolution of biomolecules. Nature 472, 499-503.
Esvelt, K. M., Mali, P., Braff, J. L., Moosburner, M., Yaung, S. J., and Church, G. M. (2013). Orthogonal Cas9 proteins for RNA-guided gene regulation and editing. Nature Methods 10, 1116-1121.
Farzadfard, F., Perli, S. D., and Lu, T. K. (2013). Tunable and Multifunctional Eukaryotic Transcription Factors Based on CRISPR/Cas. ACS Synthetic Biology 2, 604-613.
Feng, J., Bi, C., Clark, B. S., Mady, R., Shah, P., and Kohtz, J. D. (2006). The Evf-2 noncoding RNA is transcribed from the Dlx-5/6 ultraconserved region and functions as a Dlx-2 transcriptional coactivator. Genes & development 20, 1470-1484.
Ferre-D'Amare, A. R., Zhou, K., and Doudna, J. A. (1998). Crystal structure of a hepatitis deltavirus ribozyme. Nature 395, 567-574.
Fussenegger, M., Morris, R. P., Fux, C., Rimann, M., von Stockar, B., Thompson, C. J., and Bailey, J. E. (2000). Streptogramin-based gene regulation systems for mammalian cells. Naturebiotechnology 18, 1203-1208.
Gao, Y., and Zhao, Y. (2014). Self-processing of ribozyme-flanked RNAs into guide RNAs in vitro and in vivo for CRISPR-mediated genome editing. Journal of Integrative Plant Biology, n/an/a.
Gilbert, Luke A., Larson, Matthew H., Morsut, L., Liu, Z., Brar, Gloria A., Tones, Sandra E., Stern-Ginossar, N., Brandman, O., Whitehead, Evan H., Doudna, Jennifer A., et al. (2013). CRISPR-Mediated Modular RNA-Guided Regulation of Transcription in Eukaryotes. Cell 154, 442-451.
Gossen, M., and Bujard, H. (1992). Tight control of gene expression in mammalian cells by tetracycline-responsive promoters. Proceedings of the National Academy of Sciences 89, 5547-5551.
Greber, D., El-Baba, M. D., and Fussenegger, M. (2008). Intronically encoded siRNAs improve dynamic range of mammalian gene regulation systems and toggle switch. Nucleic Acids Res 36, e101.
Guido, N. J., Wang, X., Adalsteinsson, D., McMillen, D., Hasty, J., Cantor, C. R., Elston, T. C., and Collins, J. J. (2006). A bottom-up approach to gene regulation. Nature 439, 856-860.
Haurwitz, R. E., Sternberg, S. H., and Doudna, J. A. (2012). Csy4 relies on an unusual catalytic dyad to position and cleave CRISPR RNA. Embo j 31, 2824-2832.
Hooshangi, S., Thiberge, S., and Weiss, R. (2005). Ultrasensitivity and noise propagation in a synthetic transcriptional cascade. Proc Natl Acad Sci USA 102, 3581-3586.
Houseley, J., and Tollervey, D. (2009). The Many Pathways of RNA Degradation. Cell 136, 763-776. Jackson, R. J. (1993). Cytoplasmic regulation of mRNA function: The importance of the 3′ untranslated region. Cell 74, 9-14.
Jiang, W., Bikard, D., Cox, D., Zhang, F., and Marraffini, L. A. (2013). RNA-guided editing of bacterial genomes using CRISPR-Cas systems. Nat Biotech 31, 233-239.
Jinek, M., Chylinski, K., Fonfara, I., Hauer, M., Doudna, J. A., and Charpentier, E. (2012). A Programmable Dual-RNA-Guided DNA Endonuclease in Adaptive Bacterial Immunity. Science 337, 816-821.
Jinek, M., East, A., Cheng, A., Lin, S., Ma, E., and Doudna, J. (2013). RNA-programmed genome editing in human cells. eLife 2, e00471.
Kampf, M. M., Engesser, R., Busacker, M., Homer, M., Karlsson, M., Zurbriggen, M. D., Fussenegger, M., Timmer, J., and Weber, W. (2012). Rewiring and dosing of systems modules as a design approach for synthetic mammalian signaling networks. Molecular bioSystems 8, 1824-1832.
Kemmer, C., Fluri, D. A., Witschi, U., Passeraub, A., Gutzwiller, A., and Fussenegger, M. (2011). A designer network coordinating bovine artificial insemination by ovulation-triggered release of implanted sperms. Journal of controlled release: official journal of the Controlled Release Society 150, 23-29.
Kemmer, C., Gitzinger, M., Daoud-El Baba, M., Djonov, V., Stelling, J., and Fussenegger, M. (2010). Self-sufficient control of urate homeostasis in mice by a synthetic circuit. Naturebiotechnology 28, 355-360.
Kennedy, A. B., Liang, J. C., and Smolke, C. D. (2013). A versatile cis-blocking and transactivation strategy for ribozyme characterization. Nucleic acids research 41, e41.
Khalil, A., Lu, T. K., Bashor, C., Ramirez, C., Pyenson, N., Joung, J. K., and Collins, J. J. (2012). A Synthetic Biology Framework for Programming Eukaryotic Transcription Functions. Cell 150, 647-658.
Kim, Y.-K., and Kim, V. N. (2007). Processing of intronic microRNAs. The EMBO Journal 26, 775-783.
Koizumi, M., Soukup, G. A., Kerr, J. N., and Breaker, R. R. (1999). Allosteric selection of ribozymes that respond to the second messengers cGMP and cAMP. Nature structural biology 6, 1062-1071.
Kuwabara, T., Warashina, M., Tanabe, T., Tani, K., Asano, S., and Taira, K. (1998). A novel allosterically trans-activated ribozyme, the maxizyme, with exceptional specificity in vitro and invivo. Mol Cell 2, 617-627.
Lee, J. T. (2012). Epigenetic Regulation by Long Noncoding RNAs. Science 14, 1435-1439. Levine, J. H., Fontes, M. E., Dworkin, J., and Elowitz, M. B. (2012). Pulsed feedback defers cellular differentiation. PLoS biology 10, e1001252.
Lin, R., Maeda, S., Liu, C., Karin, M., and Edgington, T. S. (2006). A large noncoding RNA is a marker for murine hepatocellular carcinomas and a spectrum of human carcinomas. Oncogene 26, 851-858.
Lohmueller, J. J., Armel, T. Z., and Silver, P. A. (2012). A tunable zinc finger-based framework for Boolean logic computation in mammalian cells. Nucleic Acids Research.
Maeder, M. L., Linder, S. J., Cascio, V. M., Fu, Y., Ho, Q. H., and Joung, J. K. (2013a). CRISPRRNA-guided activation of endogenous human genes. Nat Methods 10, 977-979.
Maeder, M. L., Linder, S. J., Reyon, D., Angstman, J. F., Fu, Y., Sander, J. D., and Joung, J. K. (2013b). Robust, synergistic regulation of human gene expression using TALE activators. NatMeth 10, 243-245.
Maeder, M. L., Thibodeau-Beganny, S., Sander, J. D., Voytas, D. F., and Joung, J. K. (2009). Oligomerized pool engineering (OPEN): an ‘open-source’ protocol for making customized zincfinger arrays. Nat Protocols 4, 1471-1501.
Mali, P., Aach, J., Stranges, P. B., Esvelt, K. M., Moosburner, M., Kosuri, S., Yang, L., and Church, G. M. (2013a). CAS9 transcriptional activators for target specificity screening and paired nickases for cooperative genome engineering. Nat Biotechnol 31, 833-838.
Mali, P., Yang, L., Esvelt, K. M., Aach, J., Guell, M., DiCarlo, J. E., Norville, J. E., and Church, G. M. (2013b). RNA-Guided Human Genome Engineering via Cas9. Science 339, 823-826.
McMillen, D., Kopell, N., Hasty, J., and Collins, J. J. (2002). Synchronizing genetic relaxation oscillators by intercell signaling. Proceedings of the National Academy of Sciences 99, 679-684.
Mercer, T. R., Dinger, M E., and Mattick, J. S. (2009). Long non-coding RNAs: insights into functions. Nat Rev Genet 10, 155-159.
Muller, K., Engesser, R., Metzger, S., Schulz, S., Kampf, M. M., Busacker, M., Steinberg, T., Tomakidi, P., Ehrbar, M., Nagy, F., et al. (2013a). A red/far-red light-responsive bi-stable toggle switch to control gene expression in mammalian cells. Nucleic acids research 41, e77.
Muller, K., Engesser, R., Schulz, S., Steinberg, T., Tomakidi, P., Weber, C. C., Ulm, R., Timmer, J., Zurbriggen, M. D., and Weber, W. (2013b). Multi-chromatic control of mammalian gene expression and signaling. Nucleic Acids Res 41, e124.
Nagano, T., Mitchell, J. A., Sanz, L. A., Pauler, F. M., Ferguson-Smith, A. C., Feil, R., and Fraser, P. (2008). The Air Noncoding RNA Epigenetically Silences Transcription by Targeting G9a to Chromatin. Science 322, 1717-1720.
Nagarajan, V. K., Jones, C. I., Newbury, S. F., and Green, P. J. (2013). XRN 5′->3′ exoribonucleases: Structure, mechanisms and functions. Biochimica et Biophysica Acta (BBA)-Gene Regulatory Mechanisms 1829, 590-603.
Nissim, L., and Bar-Ziv, R. H. (2010). A tunable dual-promoter integrator for targeting of cancer cells. Mol Syst Biol 6, 444.
Nissim, L., Beatus, T., and Bar-Ziv, R. (2007). An autonomous system for identifying and governing a cell's state in yeast. Physical biology 4, 154-163.
Orioli, A., Pascali, C., Pagano, A., Teichmann, M., and Dieci, G. (2012). RNA polymerase III transcription control elements: Themes and variations. Gene 493, 185-194.
Pandey, R. R., Mondal, T., Mohammad, F., Enroth, S., Redrup, L., Komorowski, J., Nagano, T., Mancini-DiNardo, D., and Kanduri, C. (2008). Kcnqlotl Antisense Noncoding RNA MediatesLineage-Specific Transcriptional Silencing through Chromatin-Level Regulation. Molecular Ce1132, 232-246.
Park, S. H., Zarrinpar, A., and Lim, W. A. (2003). Rewiring MAP kinase pathways using alternative scaffold assembly mechanisms. Science 299, 1061-1064.
Peel, A. D., Chipman, A. D., and Akam, M. (2005). Arthropod segmentation: beyond the Drosophila paradigm. Nat Rev Genet 6, 905-916.
Perez-Pinera, P., Kocak, D. D., Vockley, C. M., Adler, A. F., Kabadi, A. M., Polstein, L. R., Thakore, P. I., Glass, K. A., Ousterout, D. G., Leong, K. W., et al. (2013a). RNA-guided gene activation byCRISPR-Cas9-based transcription factors. Nat Meth 10, 973-976.
Perez-Pinera, P., Ousterout, D. G., Brunger, J. M., Farin, A. M., Glass, K. A., Guilak, F, Crawford, G. E., Hartemink, A. J., and Gersbach, C. A. (2013b). Synergistic and tunable human geneactivation by combinations of synthetic transcription factors. Nat Meth 10, 239-242.
Pley, H. W., Flaherty, K. M., and McKay, D. B. (1994). Three-dimensional structure of a hammerhead ribozyme. Nature 372, 68-74. Proudfoot, N. J. (2011). Ending the message: poly(A) signals then and now. Genes and Development 25, 1770-1782.
Qi, L., Haurwitz, R. E., Shao, W., Doudna, J. A., and Arkin, A. P. (2012). RNA processing enables predictable programming of gene expression. Nat Biotech 30, 1002-1006.
R Hormes, M. H., I Oelze, P Marschall, M Tabler, F Eckstein, and G Sczakiel (1997). The subcellular localization and length of hammerhead ribozymes determine efficacy in human cells. Nucleic Acids Research 25, 769-775.
Reyon, D., Tsai, S. Q., Khayter, C., Foden, J. A., Sander, J. D., and Joking, J. K. (2012). FLASH assembly of TALENs for high-throughput genome editing. Nat Biotech 30, 460-465.
Rinaudo, K., Bleris, L., Maddamsetti, R., Subramanian, S., Weiss, R., and Benenson, Y. (2007). A universal RNAi-based logic evaluator that operates in mammalian cells. Nat Biotechnol 25, 795-801.
Rinn, J. L., Kertesz, M., Wang, J. K., Squazzo, S. L., Xu, X., Brugmann, S. A., Goodnough, L. H., Helms, J. A., Farnham, P. J., Segal, E., et al. (2007). Functional Demarcation of Active and Silent Chromatin Domains in Human HOX Loci by Noncoding RNAs. Cell 129, 1311-1323.
Rogakou, E. P., Pilch, D. R., On, A. H., Ivanova, V. S., and Bonner, W. M. (1998). DNA doublestranded breaks induce histone H2AX phosphorylation on serine 139. J Biol Chem 273, 5858-5868.
Saito, H., Fujita, Y., Kashida, S., Hayashi, K., and Inoue, T. (2011). Synthetic human cell fate regulation by protein-driven RNA switches. Nature communications 2, 160.
Saito, H., Kobayashi, T., Hara, T., Fujita, Y., Hayashi, K., Furushima, R., and Inoue, T. (2010). Synthetic translational regulation by an L7Ae-kink-turn RNP switch. Nature chemical biology 6, 71-78.
Sander, J. D., and Joung, J. K. (2014). CRISPR-Cas systems for editing, regulating and targeting genomes. Nat Biotechnol. Sanjana, N. E., Cong, L., Zhou, Y., Cunniff, M. M., Feng, G., and Zhang, F. (2012). A transcription activator-like effector toolbox for genome engineering. Nat Protocols 7, 171-192.
Shoval, O., and Alon, U. (2010). SnapShot: Network Motifs. Cell 143, 326-326.e321. Sinha, A., Hughes, K. R., Modrzynska, K. K., Otto, T. D., Pfander, C., Dickens, N. J., Religa, A. A., Bushell, E., Graham, A. L., Cameron, R., et al. (2014). A cascade of DNA-binding proteins for sexual commitment and development in Plasmodium. Nature 507, 253-257.
Smith, C. W. J., Porro, E. B., Patton, J. G., and Nadal-Ginard, B. (1989). Scanning from an independently specified branch point defines the 3[prime] splice site of mammalian introns. Nature 342, 243-247.
Soukup, G. A., and Breaker, R. R. (1999). Design of allosteric hammerhead ribozymes activated by ligand-induced structure stabilization. Structure 7, 783-791.
Sprinzak, D, Lakhanpal, A., LeBon, L., Garcia-Ojalvo, J., and Elowitz, M. B. (2011). Mutual inactivation of Notch receptors and ligands facilitates developmental patterning. PLoS computational biology 7, e1002069.
Sternberg, S. H., Haurwitz, R. E., and Doudna, J. A. (2012). Mechanism of substrate selection by a highly specific CRISPR endoribonuclease. Rna 18, 661-672.
Tabor, J. J., Salis, H. M., Simpson, Z. B., Chevalier, A. A., Levskaya, A., Marcotte, E. M., Voigt, C. A., and Ellington, A. D. (2009). A synthetic genetic edge detection program. Cell 137, 1272-1281.
Taggart, A. J., DeSimone, A. M., Shih, J. S., Filloux, M. E., and Fairbrother, W. G. (2012). Largescale mapping of branchpoints in human pre-mRNA transcripts in vivo. Nature structural & molecular biology 19, 719-721.
Teichmann, M., Dieci, G., Pascali, C., and Boldina, G. (2010). General transcription factors and subunits of RNA polymerase III: Paralogs for promoter- and cell type-specific transcription in multicellular eukaryotes. Transcription 1, 130-135.
Upadhyay, S. K., Kumar, J., Alok, A., and Tuli, R. (2013). RNA-Guided Genome Editing for Target Gene Mutations in Wheat. G3: Genes|Genomes|Genetics 3, 2233-2238.
Urlinger, S., Baron, U., Thellmann, M., Hasan, M. T., Bujard, H., and Hillen, W. (2000). Exploring the sequence space for tetracycline-dependent transcriptional activators: Novel mutations yield expanded range and sensitivity. Proceedings of the National Academy of Sciences 97, 7963-7968.
Wang, D., Wang, H., Brown, J., Daikoku, T., Ning, W., Shi, Q., Richmond, A., Stricter, R., Dey, S. K., and DuBois, R. N. (2006). CXCL1 induced by prostaglandin E2 promotes angiogenesis in colorectal cancer. J Exp Med 203, 941-951.
Wang, H., Yang, H., Shivalila, C. S., Dawlaty, M. M., Cheng, A. W., Zhang, F., and Jaenisch, R. (2013). One-step generation of mice carrying mutations in multiple genes by CRISPR/Cas mediated genome engineering. Cell 153, 910-918.
Wang, X., Arai, S., Song, X., Reichart, D., Du, K., Pascual, G., Tempst, P., Rosenfeld, M. G., Glass, C. K., and Kurokawa, R. (2008). Induced ncRNAs allosterically modify RNA-binding proteins in cis to inhibit transcription. Nature 454, 126-130.
Weber, W., Bacchus, W., Gruber, F., Hamberger, M., and Fussenegger, M. (2007). A novel vector platform for vitamin H-inducible transgene expression in mammalian cells. Journal of biotechnology 131, 150-158.
Weber, W., and Fussenegger, M. (2009). Engineering of Synthetic Mammalian Gene Networks. Chemistry & Biology 16, 287-297.
Weber, W., and Fussenegger, M. (2012). Emerging biomedical applications of synthetic biology. Nature reviews Genetics 13, 21-35.
Weber, W., Fux, C., Daoud-el Baba, M., Keller, B., Weber, C. C., Kramer, B. P., Heinzen, C., Aubel, D., Bailey, J. E., and Fussenegger, M. (2002). Macrolide-based transgene control in mammalian cells and mice. Nature biotechnology 20, 901-907.
Weber, W., Schoenmakers, R., Keller, B., Gitzinger, M., Grau, T., Daoud-El Baba, M., Sander, P., and Fussenegger, M. (2008). A synthetic mammalian gene circuit reveals antituberculosis compounds. Proceedings of the National Academy of Sciences of the United States of America 105, 9994-9998.
White, R. J. (1998). RNA Polymerase III Transcription (Springer-Verlag). Willis, I. M. (1993). RNA polymerase III. European Journal of Biochemistry 212, 1-11.
Wilson, R. C., and Doudna, J. A. (2013). Molecular Mechanisms of RNA Interference. AnnualReview of Biophysics 42, 217-239.
Wilusz, J. E., Freier, S. M., and Spector, D. L. (2008). 3′ End Processing of a Long Nuclear-Retained Noncoding RNA Yields a tRNA-like Cytoplasmic RNA. Cell 135, 919-932.
Wilusz, J. E., JnBaptiste, C. K., Lu, L. Y., Kuhn, C. D., Joshua-Tor, L., and Sharp, P. A. (2012). A triple helix stabilizes the 3′ ends of long noncoding RNAs that lack poly(A) tails. Genes &development 26, 2392-2407.
Win, M. N., Liang, J. C., and Smolke, C. D. (2009). Frameworks for programming biological function through RNA parts and devices. Chem Biol 16, 298-310.
Xie, Z., Wroblewska, L., Prochazka, L., Weiss, R., and Benenson, Y. (2011). Multi-input RNAi based logic circuit for identification of specific cancer cells. Science 333, 1307-1311.
Yang, H., Wang, H., Shivalila, C. S., Cheng, A. W., Shi, L., and Jaenisch, R. (2013). One-step generation of mice carrying reporter and conditional alleles by CRISPR/Cas-mediated genome engineering. Cell 154, 1370-1379.
Ye, H., Daoud-El Baba, M., Peng, R. W., and Fussenegger, M. (2011). A synthetic optogenetic transcription device enhances blood-glucose homeostasis in mice. Science 332, 1565-1568.
Yin, Q.-F., Yang, L., Zhang, Y., Xiang, J.-F., Wu, Y.-W., Carmichael, G. G., and Chen, L.-L. (2012). Long Noncoding RNAs with snoRNA Ends. Molecular cell 48, 219-230.
Ying, S.-Y., and Lin, S.-L. (2005). Intronic microRNAs. Biochemical and Biophysical Research Communications 326, 515-520.
Zhao, J., Sun, B. K., Erwin, J. A., Song, J.-J., and Lee, J. T. (2008). Polycomb Proteins Targetedby a Short Repeat RNA to the Mouse X Chromosome. Science 322, 750-756.
Auslander, S., Auslander, D., Muller, M., Wieland, M., and Fussenegger, M. (2012). Programmable single-cell mammalian biocomputers. Nature 487, 123-127.
Xie, Z., Wroblewska, L., Prochazka, L., Weiss, R., and Benenson, Y. (2011). Multi-Input RNAi-Based Logic Circuit for Identification of Specific Cancer Cells. Science 333, 1307-1311.

What is claimed is:

1. An engineered construct comprising a promoter operably linked to a nucleic acid that comprises:
(a) a nucleotide sequence encoding at least one guide RNA (gRNA); and
(b) one or more nucleotide sequences selected from (i) a nucleotide sequence encoding a protein of interest and (ii) a nucleotide sequence encoding an RNA interference molecule.

2. The engineered construct of claim 1, wherein the promoter is a RNA-polymerase-II-dependent (RNA pol II) promoter.

3. The engineered construct of claim 1 or 2, wherein the at least one gRNA is flanked by nucleotide sequences encoding ribonuclease recognition sites.

4. The engineered construct of claim 3, wherein the ribonuclease recognition sites are Csy4 ribonuclease recognition sites.

5. The engineered construct of claim 1 or 2, wherein the at least one gRNA is flanked by nucleotide sequences encoding ribozymes.

6. The engineered construct of claim 5, wherein the ribozymes are selected from a hammerhead ribozyme and a Hepatitis delta virus ribozyme.

7. The engineered construct of any one of claims 1-6, wherein the nucleotide sequence of (a) is flanked by cognate intronic splice sites.

8. An engineered construct comprising a promoter operably linked to a nucleic acid that comprises a first nucleotide sequence encoding at least one guide RNA (gRNA) flanked by ribonuclease recognition sites.

9. The engineered construct of claim 8, wherein the first nucleotide sequence is flanked by cognate intronic splice sites.

10. The engineered construct of claim 8 or 9, wherein the nucleic acid further comprises a second nucleotide sequence encoding a protein of interest, wherein the first nucleotide sequence is within the second nucleotide sequence.

11. The engineered construct of any one of claims 8-10, wherein the nucleic acid further comprise a second nucleotide sequence encoding a protein of interest, wherein the second nucleotide sequence is upstream of the first nucleotide sequence.

12. The engineered construct of any one of claims 8-11, wherein the engineered construct further comprises a nucleotide sequence encoding at least one microRNA.

13. The engineered construct of claim 12, wherein the at least one microRNA is encoded within the protein of interest.

14. The engineered construct of any one of claims 10-13, wherein the nucleic acid further comprises a third nucleotide sequence encoding a triple helix structure, wherein the third nucleotide sequence is between the second nucleotide sequence and the first nucleotide sequence.

15. The engineered construct of any one of claims 8-14, wherein the promoter is a RNA-polymerase-II-dependent (RNA pol II) promoter.

16. The engineered construct of claim 15, wherein the RNA pol II promoter is a human cytomegalovirus promoter, a human ubiquitin promoter, a human histone H2A1 promoter, or a human inflammatory chemokine CXCL1 promoter.

17. The engineered construct of any one of claims 8-16, wherein the first nucleotide sequence encodes at least two gRNAs, each gRNA flanked by ribonuclease recognition sites.

18. The engineered construct of claim 17, wherein the first nucleotide sequence encodes at least three gRNAs, each gRNA flanked by ribonuclease recognition sites.

19. The engineered construct of claim 18, wherein the first nucleotide sequence encodes at least four gRNAs, each gRNA flanked by ribonuclease recognition sites.

20. The engineered construct of claim 19, wherein the first nucleotide sequence encodes at least five gRNAs, each gRNA flanked by ribonuclease recognition sites.

21. The engineered construct of any one of claims 8-20, wherein the first nucleotide sequence encodes at least two gRNAs flanked by ribonuclease recognition sites, and wherein the gRNAs are different from each other.

22. The engineered construct of any one of claims 8-21, wherein the ribonuclease recognition sites are Csy4 ribonuclease recognition sites.

23. The engineered construct of claim 22, wherein each of the Csy4 ribonuclease recognition sites has a length of 28 nucleotides.

24. The engineered construct of claim 22 or 23, wherein the Csy4 ribonuclease recognition sites are from Pseudomonas aeruginosa.

25. The engineered construct of any one of claims 14-24, wherein the triple helix structure is encoded by a nucleotide sequence from the 3′ end of the MALAT1 locus or the 3′ end of the MENβ locus.

26. An engineered construct comprising a promoter operably linked to a nucleic acid that comprises:
a first nucleotide sequence encoding a protein of interest; and
a second nucleotide sequence encoding at least one guide RNA (gRNA) flanked by ribonuclease recognition sites, wherein the second nucleotide sequence is flanked by nucleotide sequences encoding cognate intronic splice sites and is within the first nucleotide sequence.

27. The engineered construct of claim 26, wherein the engineered construct further comprises a nucleotide sequence encoding at least one microRNA.

28. The engineered construct of claim 27, wherein the at least one microRNA is encoded within the protein of interest.

29. The engineered construct of any one of claims 26-28, wherein the nucleic acid further comprises:
a third nucleotide sequence encoding a triple helix structure; and
a fourth nucleotide sequence encoding at least one gRNA flanked by ribonuclease recognition sites, wherein the third nucleotide sequence is downstream of the first nucleotide sequence and is upstream of the fourth nucleotide sequence.

30. The engineered construct of any one of claims 26-29, wherein the promoter is a RNA-polymerase-II-dependent (RNA pol II) promoter.

31. The engineered construct of claim 30, wherein the RNA pol II promoter is a human cytomegalovirus promoter, a human ubiquitin promoter, a human histone H2A1 promoter, or a human inflammatory chemokine CXCL1 promoter.

32. The engineered construct of any one of claims 26-31, wherein the second nucleotide sequence encodes at least two gRNAs, each gRNA flanked by ribonuclease recognition sites.

33. The engineered construct of claim 32, wherein the second nucleotide sequence encodes at least three gRNAs, each gRNA flanked by ribonuclease recognition sites.

34. The engineered construct of claim 33, wherein the second nucleotide sequence encodes at least four gRNAs, each gRNA flanked by ribonuclease recognition sites.

35. The engineered construct of claim 34, wherein the second nucleotide sequence encodes at least five gRNAs, each gRNA flanked by ribonuclease recognition sites.

36. The engineered construct of any one of claims 26-35, wherein the second nucleotide sequence encodes at least two gRNAs flanked by ribonuclease recognition sites, and wherein the gRNAs are different from each other.

37. The engineered construct of any one of claims 26-36, wherein the ribonuclease recognition sites are Csy4 ribonuclease recognition sites.

38. The engineered construct of claim 37, wherein each of the Csy4 ribonuclease recognition sites has a length of 28 nucleotides.

39. The engineered construct of claim 37 or 38, wherein the Csy4 ribonuclease recognition sites are from Pseudomonas aeruginosa.

40. The engineered construct of any one of claims 26-39, wherein the cognate intronic splice sites are from a consensus intron.

41. The engineered construct of any one of claims 26-39, wherein the cognate intronic splice sites are from a HSV1 latency-associated intron.

42. The engineered nucleic acid of any one of claims 26-39, wherein the cognate intronic splice sites are from a sno-IncRNA2 intron.

43. The engineered nucleic acid of any one of claims 29-42, wherein the triple helix structure is encoded by a nucleotide sequence from the 3′ end of the MALAT1 locus or the 3′ end of the MENβ locus.

44. The engineered construct of any one of claims 29-43, wherein the fourth nucleotide sequence encodes at least two gRNAs, each gRNA flanked by ribonuclease recognition sites.

45. The engineered construct of claim 44, wherein the fourth nucleotide sequence encodes at least three gRNAs, each gRNA flanked by ribonuclease recognition sites.

46. The engineered construct of claim 45, wherein the fourth nucleotide sequence encodes at least four gRNAs, each gRNA flanked by ribonuclease recognition sites.

47. The engineered construct of claim 46, wherein the fourth nucleotide sequence encodes at least five gRNAs, each gRNA flanked by ribonuclease recognition sites.

48. The engineered construct of any one of claims 29-47, wherein the fourth nucleotide sequence encodes at least two gRNAs flanked by ribonuclease recognition sites, and wherein the gRNAs are different from each other.

49. An engineered construct comprising a promoter operably linked to a nucleic acid that comprises a first nucleotide sequence encoding at least one guide RNA (gRNA) flanked by ribozymes.

50. The engineered construct of claim 49, wherein the nucleic acid further comprise a second nucleotide sequence encoding a protein of interest, wherein the second nucleotide sequence is upstream of the first nucleotide sequence.

51. The engineered construct of claim 49 or 50, wherein the engineered construct further comprises a nucleotide sequence encoding at least one microRNA.

52. The engineered construct of claim 51, wherein the at least one microRNA is encoded within the protein of interest.

53. The engineered construct of any one of claims 50-52, wherein the nucleic acid further comprises a third nucleotide sequence encoding a triple helix structure, wherein the third nucleotide sequence is between the second nucleotide sequence and the first nucleotide sequence.

54. The engineered construct of any one of claims 49-53, wherein the promoter is a RNA-polymerase-II-dependent (RNA pol II) promoter.

55. The engineered construct of claim 54, wherein the RNA pol II promoter is a human cytomegalovirus promoter, a human ubiquitin promoter, a human histone H2A1 promoter, or a human inflammatory chemokine CXCL1 promoter.

56. The engineered construct of any one of claims 49-55, wherein the first nucleotide sequence encodes at least two gRNAs, each gRNA flanked by ribozymes.

57. The engineered construct of claim 56, wherein the first nucleotide sequence encodes at least three gRNAs, each gRNA flanked by ribozymes.

58. The engineered construct of claim 57, wherein the first nucleotide sequence encodes at least four gRNAs, each gRNA flanked by ribozymes.

59. The engineered construct of claim 58, wherein the first nucleotide sequence encodes at least five gRNAs, each gRNA flanked by ribozymes.

60. The engineered construct of any one of claims 49-59, wherein the first nucleotide sequence encodes at least two gRNAs flanked by ribozymes, and wherein the gRNAs are different from each other.

61. The engineered construct of any one of claims 49-60, wherein the ribozymes are cis-acting ribozymes.

62. The engineered construct of claim 61, wherein at least one of the cis-acting ribozymes is a hammerhead ribozyme.

63. The engineered construct of claim 62, wherein the hammerhead ribozyme is at the 5′ end of the at least one gRNA.

64. The engineered construct of claim 61, wherein at least one of the cis-acting ribozymes is a Hepatitis delta virus ribozyme.

65. The engineered construct of claim 64, wherein the Hepatitis delta virus ribozyme is at the 3′ end of the at least one gRNA.

66. The engineered construct of any one of claims 53-65, wherein the triple helix structure is encoded by a nucleotide sequence from the 3′ end of the MALAT1 locus or the 3′ end of the MENβ locus.

67. An engineered construct comprising a promoter operably linked to a nucleic acid that comprises:
a first nucleotide sequence encoding at least one RNA interference molecule within a protein of interest;
a second nucleotide sequence encoding at least one guide RNA flanked by ribonuclease recognition sites; and
a third nucleotide sequence encoding a triple helix structure, wherein the third nucleotide sequence is between the first and second nucleotide sequences.

68. An engineered construct comprising a promoter operably linked to a nucleic acid that comprises:
a first nucleotide sequence encoding at least one RNA interference molecule within a protein of interest;
a second nucleotide sequence encoding at least one guide RNA flanked by ribozymes; and
a third nucleotide sequence encoding a triple helix structure, wherein the third nucleotide sequence is between the first and second nucleotide sequences.

69. The engineered construct of claim 67 or 68, wherein the at least one RNA interference molecule is selected from a microRNA (miRNA) and a small-interfering RNA (siRNA).

70. The engineered construct of claim 69, wherein the at least one RNA interference molecule comprises at least one miRNA.

71. A vector comprising the engineered construct of any one of claims 1-70.

72. A cell comprising the engineered construct of any one of claims 1-70 or the vector of claim 71.

73. A cell comprising at least two of the engineered constructs of any one of claims 1-70 or at least two of the vectors of claim 71.

74. The cell of claim 72 or 73, wherein the cell is modified to stably express a ribonuclease.

75. The cell of claim 74, wherein the ribonuclease is a Csy4 ribonuclease.

76. The cell of any one of claims 72-75, wherein the cell is modified to stably express a Cas protein.

77. The cell of claim 76, wherein the Cas protein is a Cas nuclease.

78. The cell of claim 76, wherein the Cas nuclease is a Cas9 nuclease.

79. The cell of claim 76, wherein the Cas protein is a transcriptionally active Cas protein.

80. The cell of claim 79, wherein the transcriptionally active Cas protein is a transcriptionally active Cas9 protein.

81. The cell of any one of claims 72-80, wherein the cell further comprises an engineered nucleic acid comprising a promoter operably linked to a nucleotide sequence encoding a ribonuclease.

82. The cell of claim 81, wherein the ribonuclease is a Csy4 ribonuclease.

83. The cell of any one of claims 72-82, wherein the cell further comprises an engineered nucleic acid comprising a promoter operably linked to a nucleotide sequence encoding a Cas protein.

84. The cell of claim 83, wherein the Cas protein is a Cas nuclease.

85. The cell of claim 84, wherein the Cas nuclease is a Cas9 nuclease.

86. The cell of claim 83, wherein the Cas protein is a transcriptionally active Cas protein.

87. The cell of claim 86, wherein the transcriptionally active Cas protein is a transcriptionally active Cas9 protein.

88. The cell of any one of claims 72-87, wherein the cell further comprises at least one additional engineered nucleic acid comprising a promoter operably linked to a nucleotide sequence encoding a protein of interest.

89. The cell of claim 88, wherein the protein of interest of the at least one additional engineered nucleic acid is different from any other protein of interest of the cell.

90. The cell of any one of claims 72-89, wherein the cell is a bacterial cell.

91. The cell of any one of claims 72-89, wherein the cell is a human cell.

92. A method comprising culturing the cell of any one of claims 72-91.

93. The method of claim 92 comprising culturing the cell under conditions that permit nucleic acid expression.

94. A method of producing, modifying or rewiring a cellular genetic circuit comprising:
expressing in a cell a first engineered construct selected from the engineered construct of any one of claims 1-70; and
expressing in the cell a second engineered construct selected from the engineered construct of any one of claims 1-70, wherein at least one gRNA of the first engineered construct is complementary to and binds to a region of the promoter of the second engineered construct or to a region of an endogenous promoter.

95. The method of claim 94 further comprising expressing a third engineered construct selected from the engineered construct of any one of claims 1-70, wherein at least one gRNA of the second engineered construct is complementary to and binds to a region of the promoter of the third engineered construct or to a region of an endogenous promoter.

96. The method of claim 95 further comprising expressing at least one additional engineered nucleic acid selected from the engineered nucleic acid of any one of claims 1-70, wherein at least one gRNA of the at least one additional engineered nucleic acid is complementary to and binds to a region of the promoter of any one of the engineered nucleic acids of the cell or to a region of at least one endogenous promoter.

97. The method of any one of claims 94-96, wherein the cell is modified to stably express a Cas protein.

98. The method of claim 97, wherein the Cas protein is a Cas nuclease.

99. The method of claim 98, wherein the Cas nuclease is a Cas9 nuclease.

100. The method of claim 97, wherein the Cas protein is a transcriptionally active Cas protein.

101. The method of claim 100, wherein the transcriptionally active Cas protein is a transcriptionally active Cas9 protein.

102. The method of any one of claims 94-101, wherein the cell further comprises an engineered nucleic acid comprising a promoter operably linked to a nucleotide sequence encoding a ribonuclease.

103. The method of claim 102, wherein the ribonuclease is a Csy4 ribonuclease.

104. The method of any one of claims 94-103, wherein the cell further comprises an engineered nucleic acid comprising a promoter operably linked to a nucleotide sequence encoding a Cas protein.

105. The method of claim 104, wherein the Cas protein is a Cas nuclease.

106. The method of claim 105, wherein the Cas nuclease is a Cas9 nuclease.

107. The method of claim 104, wherein the Cas protein is a transcriptionally active Cas protein.

108. The method of claim 107, wherein the transcriptionally active Cas protein is a transcriptionally active Cas9 protein.

109. The method of any one of claims 94-108 further comprising culturing the cell.

110. A method of multiplexed cellular expression of guide ribonucleic acids (gRNAs) comprising expressing in a cell an engineered construct comprising a promoter operably linked to a nucleic acid that comprises a first nucleotide sequence encoding at least two gRNAs, each gRNA flanked by ribonuclease recognition sites.

111. The method of claim 110, wherein the nucleic acid further comprises a second nucleotide sequence encoding a protein of interest, wherein the second nucleotide sequence is upstream of the first nucleotide sequence.

112. The method of claim 111, wherein the engineered construct further comprises a nucleotide sequence encoding at least one microRNA.

113. The engineered construct of claim 112, wherein the at least one microRNA is encoded within the protein of interest.

114. The method of any one of claims 111-113, wherein the nucleic acid further comprises a third nucleotide sequence encoding a triple helix structure, wherein the third nucleotide sequence is between the second nucleotide sequence and the first nucleotide sequence.

115. The method of any one of claims 110-114, wherein the cell is modified to stably express a Cas protein.

116. The method of claim 115, wherein the Cas protein is a Cas nuclease.

117. The method of claim 116, wherein the Cas nuclease is a Cas9 nuclease.

118. The method of claim 115, wherein the Cas protein is a transcriptionally active Cas protein.

119. The method of claim 118, wherein the transcriptionally active Cas protein is a transcriptionally active Cas9 protein.

120. The method of any one of claims 110-119, wherein the cell further comprises an engineered nucleic acid comprising a promoter operably linked to a nucleotide sequence encoding a ribonuclease.

121. The method of claim 120, wherein the ribonuclease is a Csy4 ribonuclease.

122. The method of any one of claims 110-121, wherein the cell further comprises an engineered nucleic acid comprising a promoter operably linked to a nucleotide sequence encoding a Cas protein.

123. The method of claim 122, wherein the Cas protein is a Cas nuclease.

124. The method of claim 123, wherein the Cas nuclease is a Cas9 nuclease.

125. The method of claim 122, wherein the Cas protein is a transcriptionally active Cas protein.

126. The method of claim 125, wherein the transcriptionally active Cas protein is a transcriptionally active Cas9 protein.

127. The method of any one of claims 110-126 further comprising culturing the cell.


SEQUENCE LISTING (25 sequences)

SEQ ID NO: 1 - DNA, 110 bp, Artificial Sequence
  Synthetic Polynucleotide
gattcgtcagtagggttgtaaaggtttttcttttcctgagaaaacaaccttttgttttctcaggttttgctttttggcctttccctagctttaaaaaaaaaaaagcaaaa

SEQ ID NO: 2 - DNA, 4419 bp, Artificial Sequence
  Synthetic Polynucleotide
gccaccatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgacgggccctcactgggttcagggtcacccaagaagaaacgcaaagtcgaggatccaaagaagaaaaggaaggttgaagaccccaagaaaaagaggaaggtggatgggatcggctcaggcagcaacggcggtggaggttcagacgctttggacgatttcgatctcgatatgctcggttctgacgccctggatgatttcgatctggatatgctcggcagcgacgctctcgacgatttcgacctcgacatgctcgggtcagatgccttggatgattttgacctggatatgctctcatgatga

SEQ ID NO: 3 - DNA, 630 bp, Artificial Sequence
  Synthetic Polynucleotide
gccaccatgaaatcttctcaccatcaccatcaccatgaaaacctgtacttccaatccaatgcagctagcgaccactatctggacatcagactgaggcccgatcctgagttccctcccgcccagctgatgagcgtgctgtttggcaagctgcatcaggctctggtcgcccaaggcggagacagaatcggcgtgtccttccccgacctggacgagtcccggagtcgcctgggcgagcggctgagaatccacgccagcgcagacgatctgcgcgccctgctggcccggccttggctggagggcctgcgggatcatctgcagtttggcgagcccgccgtggtgccacacccaacaccctaccgccaggtgagccgcgtgcaggccaagtcaaatcccgagagactgcggcggaggctgatgaggcgacatgatctgagcgaggaggaggccagaaagagaatccccgacacagtggccagagccctggatctgccatttgtgaccctgcggagccagagcactggccagcatttcagactgttcatcagacacgggcccctgcaggtgacagccgaggagggcggatttacatgctatggcctgtctaaaggcggcttcgtgccctggttctga

SEQ ID NO: 4 - DNA, 1105 bp, Artificial Sequence
  Synthetic Polynucleotide
gccaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattgataaaccggtgattcgtcagtagggttgtaaaggtttttcttttcctgagaaaacaaccttttgttttctcaggttttgctttttggcctttccctagctttaaaaaaaaaaaagcaaaactcaccgaggcagttccataggatggcaagatcctggtattggtctgcgagttcactgccgtataggcagctaagaaatagtcgcgtgtagcgaagcagttttagagctagaaatagcaagttaaaataaggctagtccgttatcaacttgaaaaagtggcaccgagtcggtgctttttttcgttcactgccgtataggcagctaagaaacaaacaggaatcgaatgcaaccggcgcaggaacactgccagcgcatcaaccccggg

SEQ ID NO: 5 - DNA, 919 bp, Artificial Sequence
  Synthetic Polynucleotide
gccaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgaggtaagtgttcactgccgtataggcagctaagaaatagtcgcgtgtagcgaagcagttttagagctagaaatagcaagttaaaataaggctagtccgttatcaacttgaaaaagtggcaccgagtcggtgctttttttcgttcactgccgtataggcagctaagaaagagggagtcgagtcttctttttttttttcacagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattga

SEQ ID NO: 6 - DNA, 1148 bp, Artificial Sequence
  Synthetic Polynucleotide
gctagccatgcttcgctacacgcgactattaatattttcaggctagccatgcttcgctacacgcgactattaatattttcaggctagccatgcttcgctacacgcgactattaatattttcaggctagccatgcttcgctacacgcgactattaatattttcaggctagccatgcttcgctacacgcgactattaatattttcaggctagccatgcttcgctacacgcgactattaatattttcaggctagccatgcttcgctacacgcgactattaatattttcaggctagccatgcttcgctacacgcgactattaatattttcaggctagccatgcttcgctacacgcgactattaatattttcaggctagcggggggctataaaagggggtgggggcgttcgtcctgctatctagcgtcgcgttgaccatggcgccaccatgagcagcggcgccctgctgttccacggcaagatcccctacgtggtggagatggagggcgatgtggatggccacaccttcagcatccgcggtaagggctacggcgatgccagcgtgggcaaggtggatgcccagttcatctgcaccaccggcgatgtgcccgtgccctggagcaccctggtgaccaccctgacctacggcgcccagtgcttcgccaagtacggccccgagctgaaggatttctacaagagctgcatgcccgatggctacgtgcaggagcgcaccatcaccttcgagggcgatggcaatttcaagacccgcgccgaggtgaccttcgagaatggcagcgtgtacaatcgcgtgaagctgaatggccagggcttcaagaaggatggccacgtgctgggcaagaatctggagttcaatttcaccccccactgcctgtacatctggggcgatcaggccaatcacggcctgaagagcgccttcaagatctgccacgagatcgccggcagcaagggcgatttcatcgtggccgatcacacccagatgaatacccccatcggcggcggccccgtgcacgtgcccgagtaccaccacatgagctaccacgtgaagctgagcaaggatgtgaccgatcaccgcgataatatgagcctgacggagaccgtgcgcgccgtggattgccgcaagacctacctgtaa

SEQ ID NO: 7 - DNA, 1111 bp, Artificial Sequence
  Synthetic Polynucleotide
gctagcccaggacagtactccgacttacttaatattttcaggctagcccaggacagtactccgacttacttaatattttcaggctagcccaggacagtactccgacttacttaatattttcaggctagcccaggacagtactccgacttacttaatattttcaggctagcccaggacagtactccgacttacttaatattttcaggctagcccaggacagtactccgacttacttaatattttcaggctagcccaggacagtactccgacttacttaatattttcaggctagcccaggacagtactccgacttacttaatattttcaggctagcggggggctataaaagggggtgggggcgttcgtcctgctatctagcgtcgcgttgaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 8 - DNA, 926 bp, Artificial Sequence
  Synthetic Polynucleotide
gccaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgaggtaagtgttcactgccgtataggcagctaagaaatagtcgcgtgtagcgaagcagttttagagctagaaatagcaagttaaaataaggctagtccgttatcaacttgaaaaagtggcaccgagtcggtgctttttttcgttcactgccgtataggcagctaagaaatactaacttcgagtcttctttttttttttcacagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattgacccggg

SEQ ID NO: 9 - DNA, 1253 bp, Artificial Sequence
  Synthetic Polynucleotide
gccaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgaggtaagtgttcatttctcaaaagaccctaatgttcttcctttacaggaatgaatactgtgcatggaccaatgatgacttccatacatgcattccttggaaagctgaacaaaatgagtgggaactctgtactatcatcttagttgaactgaggtccggatccgttcactgccgtataggcagctaagaaatagtcgcgtgtagcgaagcagttttagagctagaaatagcaagttaaaataaggctagtccgttatcaacttgaaaaagtggcaccgagtcggtgctttttttcagatctgttcactgccgtataggcagctaagaaatctagatggatcgatgatgacttccatatatacattccttggaaagctgaacaaaatgagtgaaaactctataccgtcattctcgtcgaactgaggtccaaccggtgcacattactccaacaggggctagacagagagggccaacattgattcgttgacatgggtggctgcagtactaacttcgagtcttctttttttttttcacagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattga

SEQ ID NO: 10 - DNA, 1056 bp, Artificial Sequence
  Synthetic Polynucleotide
gccaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattgataaaccggtgattcgtcagtagggttgtaaaggtttttcttttcctgagaaaacaaccttttgttttctcaggttttgctttttggcctttccctagctttaaaaaaaaaaaagcaaaacgactactgatgagtccgtgaggacgaaacgagtaagctcgtctagtcgcgtgtagcgaagcagttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggaccccggg

SEQ ID NO: 11 - DNA, 946 bp, Artificial Sequence
  Synthetic Polynucleotide
gccaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattgataaaccggtcgactactgatgagtccgtgaggacgaaacgagtaagctcgtctagtcgcgtgtagcgaagcagttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggaccccggg

SEQ ID NO: 12 - DNA, 217 bp, Artificial Sequence
  Synthetic Polynucleotide
cgactactgatgagtccgtgaggacgaaacgagtaagctcgtctagtcgcgtgtagcgaagcagttttagagctagaaatagcaagttaaaataaggctagtccgttatcaacttgaaaaagtggcaccgagtcggtgcttttggccggcatggtcccagcctcctcgctggcgccggctgggcaacatgcttcggcatggcgaatgggaccccggg

SEQ ID NO: 13 - DNA, 1470 bp, Artificial Sequence
  Synthetic Polynucleotide
gccaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattgataaaccggtgattcgtcagtagggttgtaaaggtttttcttttcctgagaaaacaaccttttgttttctcaggttttgctttttggcctttccctagctttaaaaaaaaaaaagcaaaactcaccgaggcagttccataggatggcaagatcctggtatcggtctgcgagttcactgccgtataggcagctaagaaagctagcgtgtactctctgaggtgctcgttttagagctagaaatagcaagttaaaataaggctagtccgttatcaacttgaaaaagtggcaccgagtcggtgctttttttcgttcactgccgtataggcagctaagaaaaggtgacgcagataagaaccagttgttttagagctagaaatagcaagttaaaataaggctagtccgttatcaacttgaaaaagtggcaccgagtcggtgctttttttcgttcactgccgtataggcagctaagaaacagggcatcaagtcagccatcagcgttttagagctagaaatagcaagttaaaataaggctagtccgttatcaacttgaaaaagtggcaccgagtcggtgctttttttcgttcactgccgtataggcagctaagaaaagtcgggagtcaccctcctggaaacgttttagagctagaaatagcaagttaaaataaggctagtccgttatcaacttgaaaaagtggcaccgagtcggtgctttttttcgttcactgccgtataggcagctaagaaacccggg

SEQ ID NO: 14 - DNA, 1532 bp, Artificial Sequence
  Synthetic Polynucleotide
gccaccatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgaggtaagtgtgctcgcttcggcagcacatatactatgttgaatgaggcttcagtactttacagaatcgttgcctgcacatcttggaaacacttgctgggattacttcttcaggttaacccaacagaaggctcgagtgctgttgacagtgagcgccgcttgaagtctttaattaaatagtgaagccacagatgtatttaattaaagacttcaagcggtgcctactgcctcggagaattcaaggggctactttaggagcaattatcttgtttactaaaactgaataccttgctatctctttgatacatttttacaaagctgaattaaaatggtataaattaaatcacttttttcaattgtactaacttcgagtcttctttttttttttcacagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattgataaaccggtgattcgtcagtagggttgtaaaggtttttcttttcctgagaaaacaaccttttgttttctcaggttttgctttttggcctttccctagctttaaaaaaaaaaaagcaaaactcaccgaggcagttccataggatggcaagatcctggtatcggtctgcgagttcactgccgtataggcagctaagaaatagtcgcgtgtagcgaagcagttttagagctagaaatagcaagttaaaataaggctagtccgttatcaacttgaaaaagtggcaccgagtcggtgctttttttcccgcttgaagtctttaattaaaccgcttgaagtctttaattaaaccgcttgaagtctttaattaaaccgcttgaagtctttaattaaagttcactgccgtataggcagctaagaaacccggg

SEQ ID NO: 15 - DNA, 1154 bp, Artificial Sequence
  Synthetic Polynucleotide
gccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacgccatcagcgacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaaaccggtgattcgtcagtagggttgtaaaggtttttcttttcctgagaaaacaaccttttgttttctcaggttttgctttttggcctttccctagctttaaaaaaaaaaaagcaaaactcaccgaggcagttccataggatggcaagatcctggtatcggtctgcgagttcactgccgtataggcagctaagaaaccgcttgaagtctttaattaaaccgcttgaagtctttaattaaaccgcttgaagtctttaattaaaccgcttgaagtctttaattaaacctctggccacatcggttcctgctccgcttgaagtctttaattaaaccgcttgaagtctttaattaaaccgcttgaagtctttaattaaaccgcttgaagtctttaattaaagttcactgccgtataggcagctaagaaacccggg

SEQ ID NO: 16 - DNA, 20 bp, Artificial Sequence
  Synthetic Polynucleotide
gagtcgcgtgtagcgaagca

SEQ ID NO: 17 - DNA, 20 bp, Artificial Sequence
  Synthetic Polynucleotide
gtaagtcggagtactgtcct

SEQ ID NO: 18 - DNA, 20 bp, Artificial Sequence
  Synthetic Polynucleotide
gtgtactctctgaggtgctc

SEQ ID NO: 19 - DNA, 20 bp, Artificial Sequence
  Synthetic Polynucleotide
gacgcagataagaaccagtt

SEQ ID NO: 20 - DNA, 20 bp, Artificial Sequence
  Synthetic Polynucleotide
gcatcaagtcagccatcagc

SEQ ID NO: 21 - DNA, 20 bp, Artificial Sequence
  Synthetic Polynucleotide
ggagtcaccctcctggaaac

SEQ ID NO: 22 - DNA, 20 bp, Artificial Sequence
  Synthetic Polynucleotide
ggaatccatggagggaagat

SEQ ID NO: 23 - DNA, 20 bp, Artificial Sequence
  Synthetic Polynucleotide
tgttctcgctcaggtcagtg

SEQ ID NO: 24 - DNA, 20 bp, Artificial Sequence
  Synthetic Polynucleotide
caatgaccccttcattgacc

SEQ ID NO: 25 - DNA, 20 bp, Artificial Sequence
  Synthetic Polynucleotide
ttgattttggagggatctcg